SEQUENCE LISTING (20 sequences)

SEQ ID NO: 1 - protein, 125 aa, synthetic construct
  REGION 1..125: note = Synthetic
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 2 - protein, 8 aa, synthetic construct
  REGION 1..8: note = Synthetic
GFTFSNFW

SEQ ID NO: 3 - protein, 8 aa, synthetic construct
  REGION 1..8: note = Synthetic
MNQDGSEK

SEQ ID NO: 4 - protein, 18 aa, synthetic construct
  REGION 1..18: note = Synthetic
ARDREYCISTSCYDDFDY

SEQ ID NO: 5 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 6 - protein, 6 aa, synthetic construct
  REGION 1..6: note = Synthetic
QSISSY

SEQ ID NO: 7 - skipped

SEQ ID NO: 8 - protein, 10 aa, synthetic construct
  REGION 1..10: note = Synthetic
QQSYSTPPIT

SEQ ID NO: 9 - protein, 124 aa, synthetic construct
  REGION 1..124: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 10 - protein, 8 aa, synthetic construct
  REGION 1..8: note = Synthetic
GFTFDDYS

SEQ ID NO: 11 - protein, 8 aa, synthetic construct
  REGION 1..8: note = Synthetic
ISWNSGSK

SEQ ID NO: 12 - protein, 17 aa, synthetic construct
  REGION 1..17: note = Synthetic
AKYGSGYGKFYHYGLDV

SEQ ID NO: 13 - protein, 451 aa, synthetic construct
  REGION 1..451: note = Synthetic
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 450 aa, synthetic construct
  REGION 1..450: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 15 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 18 - protein, 329 aa, synthetic construct
  REGION 1..329: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 329 aa, synthetic construct
  REGION 1..329: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 20 - protein, 107 aa, synthetic construct [sequence omitted]

(19) United States
(12) Patent Application Publication
(10) Pub. No.: US 2025/0041411 A1
(43) Pub. Date: Feb. 6, 2025

(54) Stabilized Formulations Containing Anti-BCMA x Anti-CD3 Bispecific Antibodies

(71) Applicant: Regeneron Pharmaceuticals, Inc., Tarrytown, NY (US)

(72) Inventors: Kenneth S. Graham, Pleasant Valley, NY (US); Saurabh Wadhwa, Chester Springs, PA (US); Douglas Kamen, Poughquag, NY (US)

(21) Appl. No.: 18/765,008

(22) Filed: Jul. 5, 2024

Related U.S. Application Data
(60) Provisional application No. 63/525,652, filed on Jul. 7, 2023.
(60) Provisional application No. 63/613,540, filed on Dec. 21, 2023.

(51) Int. Cl. A61K 39/395; A61K 9/08; A61K 47/18; A61K 47/26; C07K 16/28

(52) U.S. Cl. CPC A61K 39/39591; A61K 9/08; A61K 47/183; A61K 47/26; C07K 16/2809; C07K 16/2878; C07K 2317/31

(57) ABSTRACT

The present invention provides stable liquid pharmaceutical formulations comprising a human bispecific antibody that specifically binds to human BCMA and human CD3. In certain embodiments, the formulations contain, in addition to the bispecific antibody, a buffer, a surfactant, and a sugar. The pharmaceutical formulations of the present invention exhibit a substantial degree of antibody stability upon stress and storage.

### CROSS-REFERENCE TO RELATED APPLICATIONS

This application claims the benefit under 35 USC § 119(e) of US Provisional Application Nos. 63/525,652, filed Jul. 7, 2023; and 63/613,540, filed Dec. 21, 2023, each of which is incorporated herein by reference in its entirety for all purposes.

### REFERENCE TO A SEQUENCE LISTING

This application incorporates by reference a computer readable Sequence Listing in ST.26 XML format, titled 10853US01_Sequence, created on Jul. 5, 2024 and containing 27,595 bytes.

### FIELD OF THE INVENTION

The present invention relates to the field of therapeutic antibody formulations. More specifically, the present invention relates to the field of pharmaceutical formulations comprising a human bispecific antibody that specifically binds to human BCMA and human CD3.

### BACKGROUND

Therapeutic macromolecules (e.g., antibodies) must be formulated in a manner that not only makes the molecules suitable for administration to patients, but also maintains their stability during storage and subsequent use. For example, therapeutic antibodies in liquid solution are prone to degradation, aggregation and/or undesired chemical modifications unless the solution is formulated properly. The stability of an antibody in liquid formulation depends not only on the kinds of excipients used in the formulation, but also on the amounts and proportions of the excipients relative to one another. Furthermore, other considerations aside from stability must be taken into account when preparing a liquid antibody formulation. Examples of such additional considerations include the concentration of antibody that can be accommodated by a given formulation, and the visual quality or appeal of the formulation. Thus, when formulating a therapeutic antibody, great care must be taken to arrive at a formulation that remains stable, contains an adequate concentration of antibody, and possesses other properties which enable the formulation to be conveniently administered to patients.

B-cell maturation antigen (BCMA), also known as TNFRSF17, or CD269, is a type Ill transmembrane protein lacking a signal peptide and containing a cysteine-rich extracellular domain. BCMA, along with closely related proteins, promotes B-cell survival at distinct stages of development. BCMA is expressed exclusively in B-cell lineage cells, particularly in the interfollicular region of the germinal center as well as on plasmablasts and differentiated plasma cells. BCMA is selectively induced during plasma cell differentiation, and is required for optimal survival of long-lived plasma cells in the bone marrow. In multiple myeloma, BCMA is widely expressed on malignant plasma cells at elevated levels, and BCMA expression is increased with progression from normal cells to active multiple myeloma. BCMA is also expressed in other B-cell malignancies, including Waldenström's macroglobulinemia, Burkitt lymphoma, and Diffuse Large B-Cell Lymphoma. CD3 is a homodimeric or heterodimeric antigen expressed on T cells in association with the T cell receptor complex (TCR) and is required for T cell activation.

Bispecific antibodies to human BCMA and human CD3 are one example of therapeutically relevant macromolecules that require proper formulation. Such antibodies are clinically useful for, e.g., the treatment of cancer (e.g., BCMA-expressing cancers, multiple myeloma).

Although anti-BCMA×anti-CD3 bispecific antibodies are known in the art (see, e.g., WO 2020/018820), there remains a need for pharmaceutical formulations comprising anti-BCMA×anti-CD3 bispecific antibodies that are sufficiently stable and suitable for administration to patients.

### BRIEF SUMMARY OF THE INVENTION

Stable liquid pharmaceutical formulations comprising a bispecific anti-BCMA×anti-CD3 antibody and one or more excipients, as well as kits, unit dosage forms, and containers comprising such formulations and uses thereof, are provided.

In one aspect, the present invention provides a stable liquid pharmaceutical formulation comprising: (a) a bispecific antibody comprising a first antigen-binding domain that binds specifically to human BCMA and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 2, 3 and 4, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 6, 7 and 8; (b) a buffer comprising histidine; (c) an organic co-solvent comprising polysorbate; and (d) a stabilizer comprising a sugar, wherein the formulation has a pH of 6.0±0.3.

In some embodiments, the antibody concentration is from 1 mg/ml±0.1 mg/ml to 200 mg/ml±20 mg/ml. In some cases, the antibody concentration is from 2 mg/ml±0.2 mg/ml to 125 mg/ml±12.5 mg/ml. In some cases, the antibody concentration is 2 mg/ml±0.2 mg/ml. In some cases, the antibody concentration is 10 mg/ml±1 mg/ml. In some cases, the antibody concentration is 20 mg/ml±2 mg/ml. In some cases, the antibody concentration is 120 mg/ml±12.0 mg/ml.

In some embodiments, the histidine buffer concentration is from 2 mM±0.4 mM to 20 mM±4 mM. In some cases, the histidine buffer concentration is from 5 mM±1 mM to 15 mM±3 mM. In some cases, the histidine buffer concentration is from 8 mM to 12 mM. In some cases, the histidine buffer concentration is 10 mM±2 mM.

In some embodiments, wherein the polysorbate concentration is from 0.01%±0.005% to 0.5%±0.25% w/v. In some cases, the polysorbate concentration is from 0.05%±0.025% to 0.15%±0.025% w/v. In some cases, the polysorbate concentration if from 0.05% to 0.15% w/v. In some cases, the polysorbate concentration is 0.1%±0.05% w/v. In some embodiments, the polysorbate is polysorbate 80.

In some embodiments, the sugar is sucrose. In some embodiments, the sucrose concentration is from 5%±1% to 20%±4% w/v. In some cases, the sucrose concentration is from 7%±0.5% to 12%±0.5% w/v. In some cases, the sucrose concentration is from 8% to 12% w/v. In some cases, wherein the sucrose concentration is 10%±2% w/v.

In some embodiments, the pharmaceutical formulation comprises: (a) 2 mg/ml±0.4 mg/ml antibody, (b) from 5 mM±1 mM to 15 mM±3 mM histidine buffer, (c) from 0.05%±0.025% to 0.15%±0.025% w/v polysorbate, and (d) from 5%±1% to 15%±3% w/v sucrose, at pH 6.0±0.3.

In some embodiments, the pharmaceutical formulation comprises: (a) 2 mg/ml±0.4 mg/ml antibody, (b) 10 mM±2 mM histidine buffer, (c) 0.1%±0.05% w/v polysorbate, and (d) 10%±2% w/v sucrose, at pH 6.0±0.3.

In some embodiments, the pharmaceutical formulation comprises: (a) 10 mg/ml±1 mg/ml antibody, (b) from 5 mM±1 mM to 15 mM±3 mM histidine buffer, (c) from 0.05%±0.025% to 0.15%±0.025% w/v polysorbate, and (d) from 5%±1% to 15%±3% w/v sucrose, at pH 6.0±0.3.

In some embodiments, the pharmaceutical formulation comprises: (a) 10 mg/ml±1 mg/ml antibody, (b) 10 mM±2 mM histidine buffer, (c) 0.1%±0.05% w/v polysorbate, and (d) 10%±2% w/v sucrose, at pH 6.0±0.3.

In some embodiments, the pharmaceutical formulation comprises: (a) 20 mg/ml±2 mg/ml antibody, (b) from 5 mM±1 mM to 15 mM±3 mM histidine buffer, (c) from 0.05%±0.025% to 0.15%±0.025% w/v polysorbate, and (d) from 5%±1% to 15%±3% w/v sucrose, at pH 6.0±0.3.

In some embodiments, the pharmaceutical formulation comprises: (a) 20 mg/ml±2 mg/ml antibody, (b) 10 mM±2 mM histidine buffer, (c) 0.1%±0.05% w/v polysorbate, and (d) 10%±2% w/v sucrose, at pH 6.0±0.3.

In some embodiments, the pharmaceutical formulation comprises: (a) 120 mg/ml±12 mg/ml antibody, (b) from 5 mM±1 mM to 15 mM±3 mM histidine buffer, (c) from 0.05%±0.025% to 0.15%±0.025% w/v polysorbate, and (d) from 5%±1% to 15%±3% w/v sucrose, at pH 6.0±0.3.

In some embodiments, the pharmaceutical formulation comprises: (a) 120 mg/ml±12 mg/ml antibody, (b) 10 mM±2 mM histidine buffer, (c) 0.1%±0.05% w/v polysorbate, and (d) 10%±2% w/v sucrose, at pH 6.0±0.3.

In any of the various embodiments discussed above or herein, the polysorbate may be polysorbate 80.

In some embodiments, the pharmaceutical formulation comprises 2 mg/ml bispecific antibody, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists essentially of 2 mg/ml bispecific antibody, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists of 2 mg/ml bispecific antibody, 0.7 mg/ml histidine, 1.1 mg/ml L-histidine hydrochloride, 100 mg/ml sucrose, 1 mg/ml polysorbate 80, and water for injection.

In some embodiments, the pharmaceutical formulation comprises 20 mg/ml bispecific antibody, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists essentially of 20 mg/ml bispecific antibody, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists of 20 mg/ml bispecific antibody, 0.7 mg/ml histidine, 1.1 mg/ml L-histidine hydrochloride, 100 mg/ml sucrose, 1 mg/ml polysorbate 80, and water for injection.

In any of the various embodiments discussed above or herein, the formulation contains no more than 2% high molecular weight (HMW) species after 6 months of storage at 5° C., as determined by SE-UPLC.

In any of the various embodiments discussed above or herein, the formulation contains no more than 4% high molecular weight (HMW) species after 6 months of storage at 25° C. and 60% relative humidity, as determined by SE-UPLC.

In any of the various embodiments discussed above or herein, the formulation contains no more than 1.5% high molecular weight (HMW) species after 9 months of storage at −30° C., as determined by SE-UPLC.

In any of the various embodiments discussed above or herein, the formulation contains no more than 1.5% high molecular weight (HMW) species after 9 months of storage at −80° C., as determined by SE-UPLC.

In any of the various embodiments discussed above or herein, the first antigen-binding domain comprises a HCVR with at least 90% identity to the amino acid sequence of SEQ ID NO: 1 and a LCVR with at least 90% identity to the amino acid sequence of SEQ ID NO: 5, and the second antigen-binding domain comprises a HCVR with at least 90% identity to the amino acid sequence of SEQ ID NO: 9 and a LCVR with at least 90% identity to the amino acid sequence of SEQ ID NO: 5. In some cases, the first antigen-binding domain comprises a HCVR with at least 95% identity to the amino acid sequence of SEQ ID NO: 1 and a LCVR with at least 95% identity to the amino acid sequence of SEQ ID NO: 5, and the second antigen-binding domain comprises a HCVR with at least 95% identity to the amino acid sequence of SEQ ID NO: 9 and a LCVR with at least 95% identity to the amino acid sequence of SEQ ID NO: 5. In some cases, the first antigen-binding domain comprises a HCVR with at least 99% identity to the amino acid sequence of SEQ ID NO: 1 and a LCVR with at least 99% identity to the amino acid sequence of SEQ ID NO: 5, and the second antigen-binding domain comprises a HCVR with at least 99% identity to the amino acid sequence of SEQ ID NO: 9 and a LCVR with at least 99% identity to the amino acid sequence of SEQ ID NO: 5.

In any of the various embodiments discussed above or herein, the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5.

In one aspect, the present invention provides a stable liquid pharmaceutical formulation comprising: (a) 2 mg/ml±0.4 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5; (b) 10 mM±2 mM histidine buffer, pH 6.0±0.3, (c) 0.1%±0.05% w/v polysorbate 80, and (d) 10%±2% w/v sucrose.

In one aspect, the present invention provides a stable liquid pharmaceutical formulation comprising: (a) 10 mg/ml±2 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5; (b) 10 mM±2 mM histidine buffer, pH 6.0±0.3, (c) 0.1%±0.05% w/v polysorbate 80, and (d) 10%±2% w/v sucrose.

In one aspect, the present invention provides a stable pharmaceutical formulation comprising: (a) 20 mg/ml±4 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5; (b) 10 mM±2 mM histidine buffer, pH 6.0±0.3, (c) 0.1%±0.05% w/v polysorbate 80, and (d) 10%±2% w/v sucrose.

In one aspect, the present invention provides a stable pharmaceutical formulation comprising: (a) 120 mg/ml±12 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5; (b) 10 mM±2 mM histidine buffer, pH 6.0±0.3, (c) 0.1%±0.05% w/v polysorbate 80, and (d) 10%±2% w/v sucrose.

In some embodiments, the antibody comprises a human IgG heavy chain constant region attached, respectively, to the HCVR of each of the first antigen-binding domain and the second antigen-binding domain. In some cases, the heavy chain constant region is of isotype IgG1. In some cases, the heavy chain constant region is of isotype IgG4.

In some embodiments, the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification. In some cases, the modification comprises a H435R substitution (EU numbering) in a heavy chain of isotype IgG1 or IgG4. In some cases, the modification comprises a H435R substitution and a Y436F substitution (EU numbering) in a heavy chain of isotype IgG1 or IgG4.

In some embodiments, the antibody comprises a heavy chain constant region comprising an amino acid sequence selected from the group consisting of SEQ ID NO: 16, SEQ ID NO: 17, SEQ ID NO: 18, and SEQ ID NO: 19.

In some embodiments, the antibody comprises a heavy chain constant region comprising the amino acid sequence of SEQ ID NO: 16 and a heavy chain constant region comprising the amino acid sequence of SEQ ID NO: 17.

In some embodiments, the antibody comprises a heavy chain constant region comprising the amino acid sequence of SEQ ID NO: 18 and a heavy chain constant region comprising the amino acid sequence of SEQ ID NO: 19.

In some embodiments, the antibody comprises a first heavy chain containing the HCVR of the first antigen-binding domain and a second heavy chain containing the HCVR of the second antigen-binding domain, wherein the first heavy chain comprises residues 1-450 of the amino acid sequence of SEQ ID NO: 13 and the second heavy chain comprises residues 1-449 of the amino acid sequence of SEQ ID NO: 14.

In some embodiments, the antibody comprises a common light chain containing the LCVR of the first and second antigen-binding domains, wherein the common light chain comprises the amino acid sequence of SEQ ID NO: 15.

A bispecific antibody comprising a first heavy chain comprising residues 1-450 of the amino acid sequence of SEQ ID NO: 13. a second heavy chain comprising residues 1-449 of the amino acid sequence of SEQ ID NO: 14, and a common light chain (i.e., paired with each of the first and second heavy chains, respectively) comprising the amino acid sequence of SEQ ID NO: 15 is linvoseltamab. In some embodiments, the pharmaceutical formulation comprises 2 mg/ml linvoseltamab, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists essentially of 2 mg/ml linvoseltamab, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists of 2 mg/ml linvoseltamab, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation comprises 20 mg/ml linvoseltamab, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists essentially of 20 mg/ml linvoseltamab, 0.7 mg/ml histidine, 1.1 mg/ml L-histidine hydrochloride, 100 mg/ml sucrose, 1 mg/ml polysorbate 80, and water for injection. In some embodiments, the pharmaceutical formulation consists of 20 mg/ml linvoseltamab, 0.7 mg/ml histidine, 1.1 mg/ml L-histidine hydrochloride, 100 mg/ml sucrose, 1 mg/ml polysorbate 80, and water for injection.

In any of the various embodiments discussed above or herein: (a) the formulation contains no more than 1.5% high molecular weight (HMW) species after 9 months of storage at −30° C. or −80 C; (b) the formulation contains no more than 2% HMW species after 6 months of storage at 5° C.; or (c) the formulation contains no more than 3% HMW species after 6 months of storage at 25° C. and 60% relative humidity, as determined by SE-UPLC.

In one aspect, the present invention provides a pharmaceutical composition, wherein the composition comprises a pharmaceutical formulation as discussed above or herein, and the composition is contained in a container. In some cases, the container is a vial. In some cases, the vial is a 5 ml, 6 ml, 10 ml or 20 ml Type 1 clear glass vial. In some cases, the container is a syringe. In some cases, the syringe is low-tungsten glass. In some cases, the container is a prefilled syringe. In some cases, the composition is contained in an autoinjector.

In one aspect, the present invention provides a kit comprising (i) a container containing a composition comprising a pharmaceutical formulation as discussed above or herein, and instructions for use of the composition. In some cases, the container is a glass vial. In some cases, the container is a prefilled syringe. In some cases, the container is an autoinjector. In some embodiments, the instructions recite subcutaneous administration of the composition. In some embodiments, the instructions recite intravenous administration of the composition.

In one aspect, the present invention provides a unit dosage form comprising a pharmaceutical formulation as discussed above or herein, wherein the antibody is present in an amount of from 0.1 mg to 500 mg. In some cases, the antibody is present in an amount of from 5 to 250 mg. In some cases, wherein the antibody is present in an amount of from 100 to 400 mg. In some cases, the unit dosage form is a glass vial. In some cases, the unit dosage form is a prefilled syringe. In some cases, the unit dosage form is an autoinjector.

In one aspect, the present invention provides a container containing a composition comprising a pharmaceutical formulation as discussed above or herein. In some cases, the container is a glass vial. In some cases, the container is a prefilled syringe. In some cases, the container is an autoinjector.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all values falling within such ranges are encompassed within the scope of the present disclosure. Each of the values discussed above or herein may be expressed with a variation of 1%, 5%, 10% or 20%. For example, a concentration of 10 mM may be expressed as 10 mM±0.1 mM (1% variation), 10 mM±0.5 mM (5% variation), 10 mM±1 mM (10% variation) or 10 mM±2 mM (20% variation).

Other embodiments will become apparent from a review of the ensuing detailed description.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value or range of values, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, exemplary methods and materials are now described. All patents, applications and non-patent publications mentioned in this specification are incorporated herein by reference in their entireties.

### Pharmaceutical Formulations

As used herein, the expression “pharmaceutical formulation” means a combination of at least one active ingredient (e.g., a bispecific anti-BCMA×anti-CD3 antibody, which is capable of exerting a biological effect in a human or non-human animal), and at least one inactive ingredient which, when combined with the active ingredient and/or one or more additional inactive ingredients, is suitable for therapeutic administration to a human or non-human animal. The term “formulation,” as used herein, means “pharmaceutical formulation” unless specifically indicated otherwise. The present invention provides pharmaceutical formulations comprising at least one therapeutic polypeptide. According to certain embodiments of the present invention, the therapeutic polypeptide is a bispecific antibody that binds specifically to human BCMA and human CD3 or an antigen-binding fragment thereof. More specifically, the present invention includes, inter alia, pharmaceutical formulations that comprise: (i) a human bispecific antibody that specifically binds to human BCMA and human CD3; (ii) a buffer comprising histidine; (iii) an organic co-solvent comprising polysorbate; and (iv) a stabilizer comprising a sugar. Additional components may be included in the formulations of the present invention if such components do not significantly interfere with the stability of the formulation. Specific exemplary components and formulations included within the present invention are described in detail below.

The pharmaceutical formulations of the present invention may, in certain embodiments, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

Bispecific Antibodies that Specifically Bind Human BCMA and Human CD3

The pharmaceutical formulations of the present invention may comprise a human bispecific antibody, or an antigen-binding fragment thereof, that binds specifically to human BCMA and human CD3.

The term “antibody,” as used herein, which includes a “bispecific antibody,” is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In certain embodiments of the invention, the anti-BCMA×anti-CD3 bispecific antibodies of the invention are human antibodies. The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences. In various embodiments, the anti-BCMA×anti-CD3 bispecific antibody is a human IgG antibody. In various embodiments, the anti-BCMA×anti-CD3 bispecific antibody is a human antibody of isotype IgG1, IgG2, IgG3 or IgG4, or mixed isotype. In some embodiments, the anti-BCMA×anti-CD3 bispecific antibody is a human IgG1 antibody (i.e., the antibody comprises a human IgG1 heavy chain constant region attached, respectively, to the HCVR of each of the first antigen-binding domain and the second antigen-binding domain). In some embodiments, the anti-BCMA×anti-CD3 bispecific antibody is a human IgG4 antibody (i.e., the antibody comprises a human IgG4 heavy chain constant region attached, respectively, to the HCVR of each of the first antigen-binding domain and the second antigen-binding domain. In any of the embodiments discussed above or herein, the anti-BCMA×anti-CD3 bispecific antibody may comprise a human kappa light chain. In any of the embodiments discussed above or herein, the anti-BCMA×anti-CD3 bispecific antibody may comprise a human lambda light chain.

In any embodiments, the bispecific antibody may include a modification in one or both heavy chains to facilitate purification of the bispecific antibody (i.e., the heterodimer) from homodimeric impurities. In some embodiments, the bispecific antibodies include first and second heavy chains (i.e., the heavy chain of the anti-BCMA binding arm, and the heavy chain of the anti-CD3 binding arm) that are identical (e.g., both of isotype IgG1 or IgG4) except for a modification in the CH3 domain of one or the other heavy chain that reduces binding of the bispecific antibody to Protein A as compared to an antibody lacking the modification. In some cases, the CH3 domain of the first heavy chain (e.g., of the anti-BCMA binding arm) binds Protein A and the CH3 domain of the second heavy chain (e.g., of the anti-CD3 binding arm) contains a mutation that reduces or abolishes Protein A binding. In some cases, the mutation is a H435R modification (by EU numbering; H95R by IMGT exon numbering). In some cases, the mutation is a H435R modification (by EU numbering; H95R by IMGT exon numbering) and a Y436F modification (by EU numbering; Y96F by IMGT). Further modifications that may be found within the second CH3 domain include: D356E, L358M, N384S, K392N, V397M, and V422I by EU (D16E, L18M, N44S, K52N, V57M, and V82I by IMGT) in the case of IgG1 CH3 domains; and Q355R, N384S, K392N, V397M, R409K, E419Q, and V422I by EU (Q15R, N44S, K52N, V57M, R69K, E79Q, and V82I by IMGT) in the case of IgG4 CH3 domains.

In any embodiments, the bispecific antibody may include a chimeric hinge. The term “chimeric hinge” is intended to include a chimeric protein comprising a first amino acid sequence derived from the hinge region of one Ig molecule and a second amino acid sequence derived from the hinge region of a different class or subclass of Ig molecule. For example, the chimeric hinge comprises, in an embodiment, a first amino acid sequence, or an “upper hinge” sequence, derived from a human IgG1 hinge region or human IgG4 hinge region, and a second amino acid sequence, or a “lower hinge” sequence, derived from a human IgG2 hinge region. In certain embodiments, the first or “upper hinge” sequence comprises amino acid residues from positions 216 to 227 according to EU numbering. In some embodiments, the second or “lower hinge” sequence comprises amino acid residues from positions 228 to 236 according to EU numbering.

The antibodies of the invention may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The terms “antigen-binding portion” or “antigen-binding fragment” of an antibody (or simply “antibody portion” or “antibody fragment”), as used herein, refer to one or more fragments of an antibody that retain the ability to specifically bind to human BCMA or human CD3.

An “isolated antibody,” as used herein, is intended to refer to an antibody that is substantially free of other antibodies having different antigenic specificities (e.g., an isolated bispecific antibody that specifically binds human BCMA and human CD3 is substantially free of antibodies that specifically bind antigens other than human BCMA and human CD3).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds human BCMA and human CD3 may, however, have cross-reactivity to other antigens, such as BCMA or CD3 molecules from other species (orthologs). In the context of the present invention, multispecific (e.g., bispecific) antibodies that bind to human BCMA and human CD3 as well as one or more additional antigens are deemed to “specifically bind” human BCMA and human CD3. Moreover, an isolated antibody may be substantially free of other cellular material and/or chemicals.

Exemplary anti-BCMA×anti-CD3 bispecific antibodies that may be included in the pharmaceutical formulations of the present invention are set forth in WO 2020/018820, the disclosure of which is incorporated by reference in its entirety.

According to certain embodiments of the present invention, the anti-BCMA×anti-CD3 bispecific antibody, or antigen-binding fragment thereof, comprises a first antigen-binding domain that specifically binds human BCMA and a second antigen-binding domain that specifically binds human CD3, in which the first antigen-binding domain comprises heavy chain complementarity determining regions (CDRs) A1-HCDR1, A1-HCDR2, and A1-HCDR3, respectively, comprising the amino acid sequences of SEQ ID NOs: 2, 3, and 4, and the second antigen-binding domain comprises heavy chain CDRs A2-HCDR1, A2-HCDR2, and A2-HCDR3, respectively, comprising the amino acid sequences of SEQ ID NOs: 10, 11, and 12. According to certain embodiments of the present invention, the anti-BCMA×anti-CD3 bispecific antibody, or antigen-binding fragment thereof, comprises common (to both the first and second antigen-binding domains) light chain complementarity determining regions LCDR1-LCDR2-LCDR3, respectively, comprising the amino acid sequences of SEQ ID NOs: 6, 7, and 8.

In certain embodiments, the anti-BCMA×anti-CD3 bispecific antibody, or antigen-binding fragment thereof, comprises a first antigen-binding domain that specifically binds human BCMA and a second antigen-binding domain that specifically binds human CD3, in which the first antigen-binding domain comprises a heavy chain variable region (HCVR) comprising the amino acid sequence of SEQ ID NO: 1, and the second antigen-binding domain comprises a HCVR comprising the amino acid sequence of SEQ ID NO: 9. In certain embodiments, the anti-BCMA×anti-CD3 bispecific antibody, or antigen-binding fragment thereof, comprises a common light chain variable region (LCVR) comprising the amino acid sequence of SEQ ID NO: 5. In certain embodiments, the anti-BCMA×anti-CD3 bispecific antibody, or antigen-binding fragment thereof, comprises a first antigen-binding domain comprising a HCVR/LCVR amino acid sequence pair comprising the amino acid sequences of SEQ ID NOs: 1/5, and a second antigen-binding domain comprising a HCVR/LCVR amino acid sequence pair comprising the amino acid sequences of SEQ ID NOs: 9/5. In some embodiments, the anti-BCMA×anti-CD3 bispecific antibody comprises the HCVR/LCVR sequence pairs noted above, and a human IgG1 heavy chain constant region. In some embodiments, the anti-BCMA×anti-CD3 bispecific antibody comprises the HCVR/LCVR sequence pairs noted above, and a human IgG4 heavy chain constant region. In some embodiments, the anti-BCMA×anti-CD3 bispecific antibody comprises the HCVR/LCVR sequence pairs noted above, and a human IgG heavy chain constant region. In some embodiments, the anti-BCMA×anti-CD3 bispecific antibody comprises the HCVR/LCVR sequence pairs noted above, and a human IgG1 or IgG4 heavy chain constant region. In some embodiments, the anti-BCMA×anti-CD3 bispecific antibody comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15 (this bispecific antibody is also referred to herein as linvoseltamab). An anti-BCMA×anti-CD3 bispecific antibody with a first antigen-binding domain that specifically binds human BCMA and comprises a HCVR comprising the amino acid sequence of SEQ ID NO: 1 and a LCVR comprising the amino acid sequence of SEQ ID NO: 5, and a second antigen-binding domain that specifically binds human CD3 and comprises a HCVR comprising the amino acid sequence of SEQ ID NO: 9 and a LCVR comprising the amino acid sequence of SEQ ID NO: 5 is REGN5458 (referred to, herein, as “mAb1”). This antibody has a first heavy chain (including the HCVR that specifically binds human BCMA) comprising the amino acid sequence of SEQ ID NO: 13, a second heavy chain (including the HCVR that specifically binds human CD3) comprising the amino acid sequence of SEQ ID NO: 14, and a common light chain comprising the amino acid sequence of SEQ ID NO: 15. In some cases, the mature form of the antibody may not include the C-terminal lysine residues of SEQ ID NOs: 13 and 14. Thus, in some cases the anti-BCMA binding arm of mAb1 comprises a heavy chain comprising residues 1-450 of SEQ ID NO: 13, and the anti-CD3 binding arm of mAb1 comprises a heavy chain comprising residues 1-449 of SEQ ID NO: 14.

The amount of antibody, or antigen-binding fragment thereof, contained within the pharmaceutical formulations of the present invention may vary depending on the specific properties desired of the formulations, as well as the particular circumstances and purposes for which the formulations are intended to be used. In certain embodiments, the pharmaceutical formulations may contain about 0.1 mg/mL to about 500 mg/mL of antibody; about 0.5 mg/mL to about 400 mg/mL of antibody; about 1 mg/mL to about 200 mg/mL of antibody; about 2 mg/mL to about 100 mg/mL; about 1 mg/mL to about 5 mg/mL of antibody; about 5 mg/mL to about 15 mg/mL of antibody; about 10 mg/mL to about 30 mg/mL of antibody; about 75 mg/mL to about 125 mg/mL; about 100 mg/mL to about 140 mg/mL of antibody; about 110 mg/mL to about 130 mg/mL of antibody; about 5 mg/mL to about 50 mg/mL; about 4 mg/ml to about 60 mg/ml; or about 2 mg/mL to about 55 mg/mL of antibody. For example, the formulations of the present invention may by liquid formulations that comprise about 0.5 mg/mL; about 1 mg/mL; about 2 mg/mL; about 3 mg/mL; about 4 mg/mL; about 5 mg/mL; about 6 mg/mL; about 7 mg/mL, about 8 mg/mL; about 9 mg/mL; about 10 mg/mL; about 11 mg/mL; about 12 mg/mL; about 13 mg/mL; about 14 mg/mL; about 15 mg/mL; about 16 mg/mL; about 17 mg/mL; about 18 mg/mL; about 19 mg/mL; about 20 mg/mL; about 21 mg/mL; about 22 mg/mL; about 23 mg/mL; about 24 mg/mL; about 25 mg/mL; about 26 mg/mL; about 27 mg/mL; about 28 mg/mL; about 29 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 96 mg/mL; about 97 mg/mL; about 98 mg/mL; about 99 mg/mL; about 100 mg/mL; about 101 mg/mL; about 102 mg/mL; about 103 mg/mL; about 104 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof, that binds specifically to human BCMA and human CD3. In certain embodiments, the pharmaceutical formulations are liquid formulations that may contain 1±0.1 mg/mL to 200±20 mg/mL of antibody; 2±0.2 mg/mL to 10±1 mg/mL of antibody; 5±0.5 mg/mL to 15±1.5 mg/mL of antibody; 1±0.5 mg/mL to 30±5 mg/mL of antibody; 40±4 mg/mL to 60±6 mg/mL of antibody; 1±0.1 mg/mL to 3±0.3 mg/mL of antibody; 3±0.5 mg/mL to 7±0.5 mg/mL of antibody; 45±1 mg/mL to 55±1 mg/mL of antibody; 110±5 mg/mL to 130±5 mg/mL of antibody; 140±5 mg/ml to 160±5 mg/ml of antibody; or 175±5 mg/mL to 185±5 mg/mL of antibody. In some embodiments, the pharmaceutical formulations contain 5±0.5 mg/mL of antibody. In some embodiments, the pharmaceutical formulations contain 10±1 mg/mL of antibody. In some embodiments, the pharmaceutical formulations contain 50±5 mg/mL of antibody. In some embodiments, the pharmaceutical formulations contain 100±10 mg/mL of antibody. In some embodiments, the pharmaceutical formulations contain 120±12 mg/mL of antibody. In some embodiments, the pharmaceutical formulations contain 150±15 mg/ml of antibody. In some embodiments, the pharmaceutical formulations contain 175±20 mg/ml of antibody.

### Bioequivalents

The present invention encompasses antibodies having amino acid sequences that vary from those of the exemplary molecules disclosed herein but that retain the ability to bind human BCMA and human CD3. Such variant molecules may comprise one or more additions, deletions, or substitutions of amino acids when compared to parent sequence, but exhibit biological activity that is essentially equivalent to that of the antibodies discussed herein.

The present invention includes antigen-binding molecules that are bioequivalent to any of the exemplary antibodies set forth herein. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antibodies are bioequivalent if there are no clinically meaningful differences in their safety, purity, and potency.

In one embodiment, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

### Formulation Excipients and pH

The pharmaceutical formulations of the present invention comprise one or more excipients. The term “excipient,” as used herein, means any non-therapeutic agent added to the formulation to provide a desired consistency, viscosity or stabilizing effect. In any of the embodiments, discussed above or herein, the formulations may also contain or comprise water for injection (USP).

In certain embodiments, the pharmaceutical formulations of the present invention comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof. In some embodiments, the sugar is sucrose. In some cases, the sugar (e.g., sucrose) acts as a thermal stabilizer for the anti-BCMA×anti-CD3 bispecific antibody.

The amount of sugar (e.g., sucrose) contained within the pharmaceutical formulations of the present invention will vary depending on the specific circumstances and intended purposes for which the formulations are used. In certain embodiments, the formulations may contain about 0.1% to about 20% sugar; about 0.5% to about 20% sugar; about 1% to about 20% sugar; about 2% to about 15% sugar; about 5% to about 15% sugar; about 7.5% to about 12.5% sugar; or about 9% to about 11% sugar. For example, the pharmaceutical formulations of the present invention may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 10.5%; about 11.0%; about 11.5%; about 12.0%; about 12.5%; about 13.0%; about 13.5%; about 14.0%; about 14.5%; about 15%; or about 20% sugar (e.g., sucrose). In some embodiments, the formulations contain about 10% sugar (e.g., sucrose). In some embodiments, the formulations contain about 5% sugar (e.g., sucrose). Each of the percentages noted above corresponds to a percent weight/volume (w/v). In some cases, the formulations contain from 5%±1% to 20%±4% w/v sucrose. In some cases, the formulations contain from 5% to 10% w/v sucrose. In some cases, the formulations contain from 8%±0.5% to 12%±0.5% w/v sucrose. In some cases, the formulations contain 10%±1% w/v sucrose. In some embodiments, the formulations contain from 90-110 mg/ml sucrose, or 95-105 mg/ml sucorse (e.g., 100 mg/ml sucrose).

The pharmaceutical formulations of the present invention may also comprise one or more organic co-solvents (or interfacial stabilizer) in a type and in an amount that stabilizes the anti-BCMA×anti-CD3 bispecific antibody under conditions of rough handling or agitation, such as, e.g., orbital shaking. In some embodiments, the organic co-solvent is a surfactant. As used herein, the term “surfactant” means a substance which reduces the surface tension of a fluid in which it is dissolved and/or reduces the interfacial tension between oil and water. Surfactants can be ionic or non-ionic. Exemplary non-ionic surfactants that can be included in the formulations of the present invention include, e.g., alkyl poly(ethylene oxide), alkyl polyglucosides (e.g., octyl glucoside and decyl maltoside), fatty alcohols such as cetyl alcohol and oleyl alcohol, cocamide MEA, cocamide DEA, and cocamide TEA. Specific non-ionic surfactants that can be included in the formulations of the present invention include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 188 (also known as Pluronic F68), poloxamer 407; polyethylene-polypropylene glycol; or polyethylene glycol (PEG). In some embodiments, the surfactant is polysorbate 80.

The amount of surfactant contained within the pharmaceutical formulations of the present invention may vary depending on the specific properties desired of the formulations, as well as the particular circumstances and purposes for which the formulations are intended to be used. In certain embodiments, the formulations may contain about 0.01% to about 1% surfactant; about 0.01% to about 0.5% surfactant; about 0.05% to about 0.15%; about 0.075% to about 0.125%; or about 0.09% to about 0.11% surfactant. For example, the formulations of the present invention may comprise about 0.01%; about 0.02%; about 0.03%; about 0.04%; about 0.05%; about 0.06%; about 0.07%; about 0.08%; about 0.09%; about 0.10%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; or about 0.30% surfactant (e.g., polysorbate 80). In some embodiments, the formulations contain about 0.1% surfactant (e.g., polysorbate 80). Each of the percentages noted above corresponds to a percent weight/volume (w/v). In some cases, the formulations contain from 0.01%±0.005% to 0.5%±0.25% w/v polysorbate 80. In some cases, the formulations contain 0.1%±0.05% w/v polysorbate 80. In some cases, the formulations contain 0.1%±0.01% w/v polysorbate 80. In some embodiments, the formulations contain 0.75-1.25 mg/ml polysorbate 80, or 0.9-1.1 mg/ml polysorbate 80 (e.g., 1.0 mg/ml polysorbate 80).

The pharmaceutical formulations of the present invention may also comprise a buffer or buffer system, which serves to maintain a stable pH and to help stabilize the anti-BCMA×anti-CD3 bispecific antibody. In some embodiments, the buffer or buffer system comprises at least one buffer that has a buffering range that overlaps fully or in part the range of pH 5.5 to 6.5. In certain embodiments, the buffer comprises a histidine buffer. In certain embodiments, the buffer (e.g., histidine) is present at a concentration of from about 1 mM to about 50 mM, about 2 mM to about 20 mM, about 5 mM to about 15 mM; about 8 mM to about 12 mM; or about 9 mM to about 11 mM. In some embodiments, the buffer (e.g., histidine) is present at a concentration of about 1 mM; about 2 mM; about 3 mM; about 4 mM; about 5 mM; about 6 mM; about 7 mM; about 8 mM; about 9 mM; about 10 mM; about 11 mM; about 12 mM; about 13 mM; about 14 mM; about 15 mM; about 16 mM; about 17 mM; about 18 mM; about 19 mM; or about 20 mM. In some cases, the formulations contain a histidine buffer at a concentration of from 5 mM±1 mM to 15 mM±3 mM. In some cases, the formulations contain a histidine buffer at a concentration of 10 mM±1 mM. In some embodiments, the formulations contain 0.5-0.9 mg/ml histidine, or 0.6-0.8 mg/ml histidine (e.g., 0.7 mg/ml histidine), and 0.9-1.3 mg/ml L-histidine hydrochloride, or 1.0-1.2 mg/ml L-histidine hydrochloride (e.g., 1.1 mg/ml L-histidine hydrochloride).

In some embodiments, the pharmaceutical formulations of the present invention may also comprise arginine. In some cases, arginine is present at a concentration of from 1 to 100 mM. In some embodiments, arginine is present at a concentration of from 25 to 100 mM or 25 to 75 mM. In some cases, the antibody is present at a concentration of from 100 mg/ml to 200 mg/ml, or 150 mg/ml±10 mg/ml to 200 mg/ml±10 mg/ml.

During the antibody purification process it may be desired or necessary to exchange one buffer for another to achieve appropriate excipient concentrations, antibody concentration, pH, etc. Buffer exchange can be accomplished, e.g., by ultrafiltration/diafiltration (UF/DF) using, e.g., a semi-permeable tangential flow filtration membrane. Use of such techniques, however, has the potential to cause the Gibbs-Donnan effect (Bolton et al., 2011, Biotechnol. Prog. 27(1):140-152). The buildup of positive charge on the product side of the membrane during protein concentration is counterbalanced electrically by the preferential movement of positive ions to the opposite side of the membrane. The potential consequence of this phenomenon is that the final concentrations of certain components (e.g., histidine) may be lower than the intended target concentrations of these components due to the electrostatic repulsion of positively charged diafiltration buffer excipients to the positively charged antibody protein during the UF/DF step. Thus, the present invention includes formulations in which the concentration of, e.g., histidine vary from the recited amounts or ranges herein due to the Gibbs-Donnan effect.

Volume exclusion describes the behavior of highly concentrated samples in which a significant portion of the total volume of the solution is taken up by the solute, especially large molecules such as proteins, excluding the solvent from this space. This then decreases the total volume of solvent available for other solutes to be dissolved in, which may result in unequal partition across the ultrafiltration membrane. Thus, the present invention includes formulations in which the concentration of, e.g., histidine may vary from the recited amounts or ranges herein due to the volume exclusion effect.

The present invention includes “buffer-free” or “self-buffering” formulations. In certain embodiments, a formulation is considered “buffer-free” or “self-buffering” if it does not include a separate buffering component. In such embodiments, the active agent/protein component (e.g., antibody) may function as a self-buffering agent, thereby maintaining the pH within a desired range under a variety of storage conditions. The present invention thus includes formulations comprising the antibody, and one or more of a tonicity agent, a stabilizer, and/or a surfactant, but without an added buffering component.

During the manufacture of the formulations of the present invention, variations in the composition of the formulation may occur. These variations may include the concentration of the active ingredient, the concentration of the excipients, and/or the pH of the formulation. The present invention includes formulations comprising anti-BCMA×anti-CD3 bispecific antibodies which are stable and retain potency with up to at least 10% variation in the excipient concentration. For example, included herein are anti-BCMA×anti-CD3 bispecific antibody formulations, wherein stability and potency of the formulations is unaffected by ±10%, or ±20% variation in the concentration of antibody, sucrose, histidine buffer and/or polysorbate 80.

### Stability of the Pharmaceutical Formulations

The pharmaceutical formulations of the present invention exhibit high levels of stability. The term “stable,” as used herein in reference to the pharmaceutical formulations, means that the antibodies within the pharmaceutical formulations retain an acceptable degree of structure and/or function and/or biological activity after storage for a defined amount of time. A formulation may be stable even though the antibody contained therein does not maintain 100% of its structure and/or function and/or biological activity after storage for a defined amount of time. Under certain circumstances, maintenance of about 90%, about 95%, about 96%, about 97%, about 98% or about 99% of an antibody's structure and/or function and/or biological activity after storage for a defined amount of time may be regarded as “stable.”

Stability can be measured by, inter alia, determining the percentage of native antibody remaining in the formulation after storage for a defined amount of time at a given temperature. The percentage of native antibody can be determined by, inter alia, size exclusion chromatography (e.g., size exclusion high performance liquid chromatography [SE-HPLC]). An “acceptable degree of stability,” as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C., or about 45° C. For example, a pharmaceutical formulation may be deemed stable if after 3 months of storage at 5° C., greater than about 90%, 95%, 96% or 97% of native antibody is detected by SE-HPLC. A pharmaceutical formulation may also be deemed stable if after 6 months of storage at 5° C., greater than about 90%, 95%, 96% or 97% of native antibody is detected by SE-HPLC. A pharmaceutical formulation may also be deemed stable if after 9 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 12 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 24 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 36 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 3 months of storage at 25° C. (and optionally 60% relative humidity), greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 6 months of storage at 25° C. (and optionally 60% relative humidity), greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 9 months of storage at 25° C. (and optionally 60% relative humidity), greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 3 months of storage at 37° C., greater than about 90%, 95%, 96% or 97% of native antibody is detected by SE-HPLC. A pharmaceutical formulation may also be deemed stable if after 1 month of storage at 45° C. (and optionally 75% relative humidity), greater than about 90%, 91%, 92%, 93%, 94%, 95%, 96%, 97% or 98% of native antibody is detected by SE-HPLC.

Other methods may be used to assess the stability of the formulations of the present invention such as, e.g., differential scanning calorimetry (DSC) to determine thermal stability, controlled agitation to determine mechanical stability, and absorbance at about 350 nm or about 405 nm to determine solution turbidities. For example, a formulation of the present invention may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at t=0.

Measuring the binding affinity of the antibody to its target may also be used to assess stability. For example, a formulation of the present invention may be regarded as stable if, after storage at e.g., −80° C., −30° C., −20° C., 5° C., 25° C., 37° C., 45° C., etc. for a defined amount of time (e.g., 14 days to 9 months), the anti-BCMA×anti-CD3 bispecific antibody contained within the formulation binds to human BCMA and human CD3 with an affinity that is at least 50%, 60%, 70%, 80%, 85%, 90%, 95%, or more of the binding affinity of the antibody prior to said storage. Binding affinity may be determined by any method, such as e.g., ELISA or plasmon resonance. Biological activity may be determined by a BCMA or CD3 activity assay, such as by contacting a cell that expresses BCMA or CD3 with the formulation comprising the anti-BCMA×anti-CD3 bispecific antibody. The binding of the antibody to such a cell may be measured directly, such as via FACS analysis.

Stability can be measured, inter alia, by determining the percentage of antibody that forms an aggregate (high molecular weight (HMW) species) within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC] or size exclusion ultra-performance liquid chromatography [SE-UPLC]). An “acceptable degree of stability”, as that phrase is used herein, means that at most 6% of the antibody is in an aggregated form detected in the formulation after storage for a defined amount of time at a given temperature (up to 25° C.). In certain embodiments an acceptable degree of stability means that at most about 10%, 9%, 8%, 7%, 6%, 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C. or about 45° C. For example, a pharmaceutical formulation may be deemed stable if after nine months of storage at 5° C., less than about 3%, 2.75%, 2.5%, 2.25%, 2%, 1.75%, 1.5%, 1.25%, 1%, 0.75%, 0.5%, 0.25%, or 0.1% of the antibody is detected in an aggregated form. In some cases, a pharmaceutical formulation may be deemed stable if after six months of storage at 5° C., less than about 5%, 4.75%, 4.5%, 4.25%, 4%, 3.75%, 3.5%, 3.25%, 2% or 1% of the antibody is detected in an aggregated form. A pharmaceutical formulation may also be deemed stable if after six months of storage at 25° C. and 60% relative humidity, less than about 6%, 5.75%, 5.5%, 5.25%, 5%, 4.5%, 4%, 3.5%, 3%, 2.5%, 2%, 1.75%, 1.5%, 1.25%, 1%, 0.75%, 0.5%, 0.25%, or 0.1% of the antibody is detected in an aggregated form. A pharmaceutical formulation may also be deemed stable if after three months of storage at 37° C., less than about 6%, 5.75%, 5.5%, 5.25%, 5%, 4.5%, 4%, 3.5%, 3%, 2.5%, 2%, 1.75%, 1.5%, 1.25%, 1%, 0.75%, 0.5%, 0.25%, or 0.1% of the antibody is detected in an aggregated form. A pharmaceutical formulation may also be deemed stable if after twelve months of storage at −30° C., or −80° C. less than about 3%, 2.75%, 2.5%, 2.25%, 2%, 1.9%, 1.8%, 1.7%, 1.6%, 1.5%, 1%, 0.5%, or 0.1% of the antibody is detected in an aggregated form.

References to stability of the pharmaceutical formulations “after” a specified period of time are intended to mean that a measurement of a stability parameter (e.g., % native form, % HMW species, or % acidic form) is taken at or about the end of the specific time period, and is not intended to mean that the pharmaceutical formulation necessarily maintains the same degree of stability for the measured parameter thereafter. For example, reference to a particular stability after 12 months means that the measurement of stability was taken at or about 12 months after the start of the study. Additional methods for assessing the stability of an antibody in formulation are demonstrated in the Examples presented below.

As illustrated in the Examples below, the present invention is based, in part, on the discovery that the combination of claimed excipients with a bispecific anti-BCMA×anti-CD3 antibody produces a formulation that is stable.

### Exemplary Formulations

According to one aspect of the present invention, the pharmaceutical formulation comprises: (i) a human anti-BCMA×anti-CD3 bispecific antibody that specifically binds to human BCMA and human CD3; (ii) a buffer comprising histidine (e.g. L-histidine); (iii) an organic co-solvent comprising polysorbate; and (iv) a stabilizer comprising a sugar. According to another aspect, the pharmaceutical formulation comprises: (i) a human anti-BCMA×anti-CD3 bispecific antibody that specifically binds to human BCMA and human CD3; (ii) a buffer comprising histidine (e.g. L-histidine); (iii) an organic co-solvent comprising polysorbate; (iv) a stabilizer comprising a sugar; and (v) arginine.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml; (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 6.0±0.3.

In some cases, the stable liquid pharmaceutical formulation comprises (i) a human bispecific antibody that specifically binds to human BCMA and human CD3 and comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15 at a concentration of from about 1 mg/ml to about 200 mg/ml; (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15 at a concentration of from about 1 mg/ml to about 200 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml; (ii) histidine at a concentration of from about 10 mM; (iii) polysorbate 80 at a concentration of from about 0.1% w/v; and (iv) sucrose at a concentration of from about 10% w/v, wherein the formulation has a pH of 6.0±0.1.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 10 mM; (iii) polysorbate 80 at a concentration of from about 0.1% w/v; and (iv) sucrose at a concentration of from about 10% w/v, wherein the formulation has a pH of 6.0±0.1.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 1 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 10 mM; (iii) polysorbate 80 at a concentration of from about 0.1% w/v; and (iv) sucrose at a concentration of from about 10% w/v, wherein the formulation has a pH of 6.0±0.1.

In some cases, the stable liquid pharmaceutical formulation comprises (i) a human bispecific antibody that specifically binds to human BCMA and human CD3 and comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15 at a concentration of from about 1 mg/ml to about 200 mg/ml; (ii) histidine at a concentration of from about 10 mM; (iii) polysorbate 80 at a concentration of from about 0.1% w/v; and (iv) sucrose at a concentration of from about 10% w/v, wherein the formulation has a pH of 6.0±0.1.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from 8 mg/ml±1 mg/ml to 12 mg/ml±1 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of 10 mg/ml±0.5 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from 110 mg/ml±5 mg/ml to 130 mg/ml±5 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of 120 mg/ml±2.5 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of 10 mg/ml±1 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of 120 mg/ml±5 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of 10 mg/ml±1 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of 120 mg/ml±5 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15 at a concentration of from 10 mg/ml±1 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15 at a concentration of 120 mg/ml±5 mg/ml; (ii) histidine at a concentration of about 10 mM±1 mM; (iii) polysorbate 80 at a concentration of about 0.1% w/v±0.01% w/v; and (iv) sucrose at a concentration of about 10% w/v±1% w/v, wherein the formulation has a pH of 6.0±0.3.

In some cases, the stable liquid pharmaceutical formulation comprises (i) a human bispecific antibody that specifically binds to human BCMA and human CD3 and comprises a first antigen-binding domain that specifically binds human BCMA comprising a HCVR comprising the amino acid sequence of SEQ ID NO: 1 and a LCVR comprising the amino acid sequence of SEQ ID NO: 5, and a second antigen-binding domain that specifically binds human CD3 comprising a HCVR comprising the amino acid sequence of SEQ ID NO: 9 and a LCVR comprising the amino acid sequence of SEQ ID NO: 5 at a concentration of from about 150 mg/ml to about 200 mg/ml; (ii) histidine at a concentration of about 10 mM±1 mM; (iii) polysorbate 80 at a concentration of about 0.1% w/v±0.01% w/v; (iv) sucrose at a concentration of about 10% w/v±1% w/v; and (v) arginine at a concentration of from 25 to 100 mM, wherein the formulation has a pH of 6.0±0.3.

In any of these exemplary formulations, “stable” may be defined as: (a) the formulation contains no more than 2.5% high molecular weight (HMW) species after 9 months of storage at 5° C., as determined by SE-UPLC; (b) the formulation contains no more than 4% high molecular weight (HMW) species after 6 months of storage at 25° C. and 60% relative humidity, as determined by SE-UPLC; (c) the formulation contains no more than 1.5% high molecular weight (HMW) species after 9 months of storage at −30° C., as determined by SE-UPLC; or (d) the formulation contains no more than 1.5% high molecular weight (HMW) species after 9 months of storage at −80° C., as determined by SE-UPLC.

In any of these exemplary formulations, the bispecific antibody may include a modification in one or both heavy chains to facilitate purification of the bispecific antibody (i.e., the heterodimer) from homodimeric impurities. In some embodiments, the bispecific antibodies include first and second heavy chains (i.e., the heavy chain of the anti-BCMA binding arm, and the heavy chain of the anti-CD3 binding arm) that are identical (e.g., both of isotype IgG1 or IgG4) except for a modification in the CH3 domain of one or the other heavy chain that reduces binding of the bispecific antibody to Protein A as compared to an antibody lacking the modification. In some cases, the CH3 domain of the first heavy chain (e.g., of the anti-BCMA binding arm) binds Protein A and the CH3 domain of the second heavy chain (e.g., of the anti-CD3 binding arm) contains a mutation that reduces or abolishes Protein A binding. In some cases, the mutation is a H435R modification (by EU numbering; H95R by IMGT exon numbering). In some cases, the mutation is a H435R modification (by EU numbering; H95R by IMGT exon numbering) and a Y436F modification (by EU numbering; Y96F by IMGT). Further modifications that may be found within the second CH3 domain include: D356E, L358M, N384S, K392N, V397M, and V422I by EU (D16E, L18M, N44S, K52N, V57M, and V82I by IMGT) in the case of IgG1 CH3 domains; and Q355R, N384S, K392N, V397M, R409K, E419Q, and V422I by EU (Q15R, N44S, K52N, V57M, R69K, E79Q, and V82I by IMGT) in the case of IgG4 CH3 domains.

Additional non-limiting examples of pharmaceutical formulations encompassed by the present invention are set forth elsewhere herein, including the working Examples presented below.

### Containers and Methods of Administration

The pharmaceutical formulations of the present invention may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present invention including, e.g., clear and opaque (e.g., amber) glass or plastic vials. Likewise, any type of syringe can be used to contain and/or administer the pharmaceutical formulations of the present invention. In some embodiments, the pharmaceutical formulation is contained in a prefilled syringe. In some embodiments, the pharmaceutical formulation is contained in a prefilled staked needle syringe.

The pharmaceutical formulations of the present invention may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present invention, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present invention, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present invention are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present invention are commercially available under the tradename “FluroTec®,” available from West Pharmaceutical Services, Inc. (Lionville, PA). FluroTec® is an example of a flurocarbon coating used to minimize or prevent drug product from adhering to the rubber surfaces. According to certain embodiments of the present invention, the pharmaceutical formulations may be contained within a low tungsten syringe that comprises a fluorocarbon-coated plunger. In some embodiments, the container is a syringe, such as an Ompi EZ-Fill™ syringe or a BD Neopak™ syringe. In some cases, the syringe is a 1 mL long glass syringe with a 1 mL iWest piston, a 27G thin wall needle and an FM30 needle shield or a BD260 needle shield. In some cases, the syringe is a 2.25 mL glass syringe with a West NovaPure™ 1-3 mL piston, a 27G thin wall needle and an FM30 needle shield or a BD260 needle shield. In various embodiments, the syringe is a 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.5 mL, 4.0 mL, 4.5 mL, 5.0 mL, 5.5 mL, 6.0 mL, 6.5 mL, 7.0 mL, 7.5 mL, 8.0 mL, 8.5 mL, 9.0 mL, 9.5 mL, or 10 mL syringe (e.g., a glass syringe).

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ 1, ∥ and Ill (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park, IL), to name only a few. In some cases, the pharmaceutical formulation is contained in a syringe specifically adapted for use with an autoinjector. Subcutaneous injections may be administered using a 20-30 gauge needle, or a 25-30 gauge needle. In some cases, subcutaneous injections may be administered using a 25 gauge needle. In some cases, subcutaneous injections may be administered using a 27 gauge needle. In some cases, subcutaneous injections may be administered using a 29 gauge needle.

Another type of delivery device can include a safety system. Such devices can be relatively inexpensive, and operate to manually or automatically extend a safety sleeve over a needle once injection is complete. Examples of safety systems can include the ERIS device by West Pharmaceutical, or the UltraSafe device by Becton Dickinson. In addition, the use of a large volume device (“LVD”), or bolus injector, to deliver the pharmaceutical formulations of the present invention is also contemplated herein. In some cases, the LVD or bolus injector may be configured to inject a medicament into a patient. For example, an LVD or bolus injector may be configured to deliver a “large” volume of medicament (typically about 2 ml to about 10 ml).

In certain embodiments, the pharmaceutical formulation is administered via an IV drip, such that the formulation is diluted in an IV bag containing a physiologically acceptable solution. In one embodiment, the pharmaceutical composition is a compounded sterile preparation in an intravenous infusion bag, such that a single dose of drug product is diluted into 100 mL, 250 mL (or other like amount suitable for intravenous drip delivery) of a physiological buffer (e.g., 0.9% saline).

The pharmaceutical formulations of the present invention can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various embodiments, the unit dosage form is contained within a container as discussed herein. Actual dosage levels of the active ingredient (e.g., an anti-BCMA×anti-CD3 bispecific antibody) in the formulations of the present invention may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various embodiments, the unit dosage form contains an amount of the active ingredient (e.g., an anti-BCMA×anti-CD3 bispecific antibody) intended for a single use. In various embodiments, the amount of the active ingredient in the unit dosage form is from about 0.1 mg to about 5000 mg, from about 100 mg to about 1000 mg, and from about 100 mg to about 500 mg, from about 100 mg to about 400 mg, from about 100 mg to about 200 mg, from about 40 mg to about 60 mg, from about 125 mg to about 175 mg, from about 160 mg to about 200 mg, from about 1 mg to about 250 mg, from about 1 mg to about 100 mg, from about 1 mg to about 50 mg, from about 1 mg to about 25 mg, from about 1 mg to about 20 mg, from about 5 mg to about 15 mg, or ranges or intervals thereof. Ranges intermediate to the above recited amounts, for example, from about 2 mg to about 100 mg or 2 mg to 20 mg, are also intended to be part of this invention. For example, ranges of values using a combination of any of the above recited values (or values contained within the above recited ranges) as upper and/or lower limits are intended to be included. In some embodiments, the unit dosage form contains 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120,130, 140, 150, 160, 170, 180, 190, or 200 mg of the antibody. In some embodiments, the unit dosage form contains 5 mg of the antibody. In some embodiments, the unit dosage form contains 25 mg of the antibody. In some embodiments, the unit dosage form contains 50 mg of the antibody. In a particular embodiment, the formulation often is supplied as a liquid in unit dosage form. In some embodiments, the unit dosage form contains from 3 to 7 mg, or from 8 to 12 mg, from 10 to 15 mg, from 35 to 45 mg, from 45 to 55 mg, from 140 to 160 mg, or from 170 to 190 mg. In some embodiments, a unit dosage form according to the present invention is suitable for subcutaneous administration to a patient (e.g., a unit dosage form containing the antibody at a concentration of from about 110 mg/ml or about 130 mg/ml, or 120 mg/ml±5 mg/ml).

The present invention also includes methods of preparing a unit dosage form. In an exemplary embodiment, a method for preparing a pharmaceutical unit dosage form includes combining the formulation of any of foregoing embodiments in a suitable container (e.g., those containers discussed herein).

### Therapeutic Uses of the Pharmaceutical Formulations

The pharmaceutical formulations of the present invention are useful, inter alia, for the treatment, prevention and/or amelioration of any disease or disorder associated with a cell expressing human BCMA. Exemplary, non-limiting diseases and disorders that can be treated by the administration of the pharmaceutical formulations of the present invention include multiple myeloma and B-cell malignancies.

The therapeutic methods of the present invention comprise administering to a subject any formulation comprising an anti-BCMA×anti-CD3 bispecific antibody as disclosed herein. The subject to which the pharmaceutical formulation is administered can be, e.g., any human or non-human animal that is in need of such treatment. For example, the subject can be an individual that is diagnosed with, or who is deemed to be at risk of being afflicted by any of the aforementioned diseases or disorders. The present invention further includes the use of any of the pharmaceutical formulations disclosed herein in the manufacture of a medicament for the treatment of any disease or disorder associated with a cell expressing human BCMA, including any of the above mentioned exemplary diseases, disorders and conditions.

In some embodiments, the present invention provides kits comprising a pharmaceutical formulation (e.g., a container with the formulation or a unit dosage form), as discussed herein, and packaging or labeling (e.g., a package insert) with instructions to use the pharmaceutical formulation for the treatment of a disease or disorder, as discussed above. In some cases, the instructions provide for use of a unit dosage form, as discussed herein, for the treatment of a disease or disorder.

A summary of the sequences and the corresponding SEQ ID NOs referenced herein is shown in Table 1, below.

TABLE 1
Summary of Sequences

| SEQ ID NO: | Description |
|---|---|
| 1 | Anti-BCMA HCVR |
| 2 | Anti-BCMA HCDR1 |
| 3 | Anti-BCMA HCDR2 |
| 4 | Anti-BCMA HCDR3 |
| 5 | Common Anti-BCMA and Anti-CD3 LCVR |
| 6 | Common Anti-BCMA and Anti-CD3 LCDR1 |
| 7 | Common Anti-BCMA and Anti-CD3 LCDR2 |
| 8 | Common Anti-BCMA and Anti-CD3 LCDR3 |
| 9 | Anti-CD3 HCVR |
| 10 | Anti-CD3 HCDR1 |
| 11 | Anti-CD3 HCDR2 |
| 12 | Anti-CD3 HCDR3 |
| 13 | Anti-BCMA Heavy Chain |
| 14 | Anti-CD3 Heavy Chain |
| 15 | Common Anti-BCMA and Anti-CD3 Light Chain |
| 16 | IgG4 Heavy Chain Constant Region |
| 17 | IgG4 Heavy Chain Constant Region with H435R/Y436F |
| 18 | IgG1 Heavy Chain Constant Region |
| 19 | IgG1 Heavy Chain Constant Region with H435R/Y436F |
| 20 | Kappa Light Chain Constant Region |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Example 1: Methods used to assess stability The physical stability of a formulation refers to properties such as color, appearance, pH, turbidity, particulates, and protein concentration. The chemical stability refers to the formation of high-molecular-weight (HMW) species, low-molecular-weight (LMW) species, charge variants, and other chemical modifications of the protein. The physical and chemical stabilities of mAb1 drug product were assessed using the following assays: Color and appearance by visual inspection; pH; Turbidity measured by increase in optical density (OD) at 405 nm; Subvisible particulate analysis by Micro-Flow Imaging™ (MFI) and light obscuration by HIAC; Protein concentration by reversed-phase ultra performance liquid chromatography (RP-UPLC); Purity of each individual drug product was assessed using size-exclusion ultra performance liquid chromatography (SE-UPLC) and Reduced and non-reduced capillary electrophoresis—sodium dodecyl sulphate (CE-SDS); Charge variant analysis was determined using cation exchange UPLC (CEX-UPLC) and imaged capillary isoelectric focusing (iCIEF); and Potency by bioassay (the relative potency of each sample was determined by bioassay and is defined as: (IC₅₀ Reference Sample/IC₅₀ Sample)×100%; the measured potency of storage stability samples must be within 50-150% of the measured potency of the reference standard). Viscosity was measured using a Rheosense Initium Viscometer. Size-exclusion ultra-performance liquid chromatography (SE-UPLC) was carried out in accordance with standard platform methods and analytical instructions. SE-UPLC data were collected on a Waters Acquity UPLC H-Class system equipped with TUV detector and 5 mm path length cell. Separation is achieved with an Acquity UPLC BEH200 SEC 1.7 mm, 4.6×300 mm column. Particle analysis was carried out in accordance with standard platform methods. A Protein Simple MF15200 instrument was used to quantify particle in the 2-10 mm size range. The instrument was equipped with a 100 mm flow cell and Bot1 autosampler.

### Example 2: Development of Stable Liquid Anti-BCMA×Anti-CD3 Bispecific Antibody Formulations

Formulation development activities involved assessment of buffers, pH, surfactants, and stabilizers to identify excipients that enhance protein stability. Additional studies were undertaken to evaluate and reduce viscosity in formulations comprising 100 mg/ml or more of mAb1. Polysorbate 80 was also ranged from 0.05% w/v to 0.2% w/v in the studies without any negative impact on formulation stability. Results generated from these studies were used to develop a formulation that was suitable for IV administration.

The effect of buffer and pH on the thermal stability of mAb1 was initially examined by incubating 5 mg/mL mAb1 at 45° C. for 28 days in a series of buffer systems at varying pH ranges. The following pH and buffer systems were studied: 20 mM acetate (pH 4.5 to 5.5) and 20 mM histidine (pH 5.5 to 6.5). Based on results from SE-UPLC and CEX-UPLC analyses, maximum protein stability was observed when mAb1 was formulated between pH 5.5 and 6.5 in histidine buffer (Table 2). These analyses also revealed that formation of low molecular weight (LMW) species (i.e., fragmentation), formation of high molecular weight (HMW) species, and generation of charge variants were the main degradation pathways. Histidine buffer and a target pH of 6.0 were selected as the formulation conditions because they provided the best overall level of protein stabilization with respect to formation of LMW and HMW species and formation of charge variants (Table 2). This target pH would also permit a suitable pH range for maximum stability that could be achieved in a large-scale manufacturing setting. Based on these results, 10 mM histidine buffer at pH 6.0 was selected for the mAb1 formulation.

Thermal stabilizers such as sucrose are often added to antibody formulations to increase the thermal stability of the protein in liquid formulations and to stabilize the protein to freezing and thawing stress. Sucrose was evaluated as a thermal stabilizer at concentrations of 5% (w/v) or 10% (w/v).

Surfactants are often added to antibody formulations to protect the protein from agitation-induced aggregation. Two surfactants, polysorbate 20 and polysorbate 80, were each evaluated at concentrations of 0.05% (w/v) and 0.1% (w/v).

To select a formulation that provided maximal stability, solutions were prepared by adding a thermal stabilizer, a surfactant or a combination of a thermal stabilizer and a surfactant to mAb1 in 10 mM Histidine, pH 6.0. Thermal stability was assessed after incubation at 45° C. for 28 days. Agitation stress stability was assessed after agitation (vortex) for 120 minutes. Freeze/thaw stability was assessed after 8 cycles of freezing and thawing. The results of freeze/thaw stability, thermal stability, and agitation stress stability studies are summarized in Tables 3-5.

After incubation at 45° C. for 28 days in the absence of sucrose, 5 mg/mL mAb1 exhibited a 0.7% increase in % HMW species as determined by SE-UPLC and a 15% increase in % acidic species as determined by CEX-UPLC. After incubation at 45° C. for 28 days, 5 mg/mL mAb1 with 10% (w/v) sucrose exhibited a 0.5% increase in % HMW species as determined by SE-UPLC and a 9.8% increase in % acidic species as determined by CEX-UPLC (Table 3). Therefore, addition of sucrose provided improved thermal stability.

mAb1 was unstable when agitated by vortexing for 120 minutes in the absence of a surfactant. Under these conditions, the solution exhibited a 35.5-48.7% increase in HMW species as determined by SE-UPLC (Table 4). The observed increase in % HMW species was greater when sucrose alone was added to 5 mg/mL mAb1. Both of the surfactants that were tested protected mAb1 from agitation-induced instability to the same extent; these formulations showed no increases in HMW species after vortexing for 120 minutes (Table 5). Additionally, no meaningful increase in % HMW species was observed in formulations containing sucrose and surfactant.

mAb1 was stable to freeze/thaw stress (Table 5).

mAb1 exhibited maximal stability when formulated in the presence of 10 mM Histidine, 0.1% (w/v) polysorbate 80, and 10% (w/v) sucrose, at pH 6.0 (Tables 3-5). The main degradation pathways identified during the development of the mAb1 liquid formulation were the formation of HMW species and charge variants.

Based on the results from formulation selection studies, mAb formulation developed to enable IV administration was an optimized, aqueous buffered formulation containing 10 mM histidine, pH 6.0, 0.1% (w/v) polysorbate 80, 10% (w/v) sucrose.

TABLE 2
Effect of Buffer and pH on the Stability of 5 mg/mL mAb1 Incubated at 45° C. for 28 Days

| Formulation | 5 mg/mL mAb1, 20 mM buffer | | | | | | | | |
|---|---|---|---|---|---|---|---|---|---|
| Fill Volume | 0.4 mL | | | | | | | | |
| Container/Closure | 2 mL Type 1 borosilicate glass vial with a FluroTec ®-coated 4432/50 butyl rubber stopper | | | | | | | | |
| | | | % Total | | | | Change in | | |
| | | Turbidity | Protein | Change in Purity | | | Charged Variants | | |
| | | (Increase | Recovered | by SE-UPLC^{a} | | | by CEX-UPLC^{a} | | |
| | Color and | in OD at | by RP- | % | % | % | % | % | % |
| pH/Buffer | Appearance | 405 nm) | UPLC | HMW | Native | LMW | Acidic | Main | Basic |
| pH 4.5, Acetate | Pass | 0.00 | 97 | 5.7 | −7.6 | 2.0 | 10.3 | −24.5 | 14.2 |
| pH 5.0, Acetate | Pass | 0.01 | 95 | 9.7 | −10.8 | 1.1 | 13.6 | −17.8 | 2.1 |
| pH 5.5, Acetate | Pass | 0.00 | 96 | 2.4 | −3.3 | 0.9 | 19.7 | −20.1 | 0.4 |
| pH 5.5, Histidine | Pass | 0.00 | 96 | 1.8 | −2.7 | 0.9 | 15.3 | −20.5 | 5.2 |
| pH 6.0, Histidine | Pass | 0.01 | 97 | 1.7 | −2.6 | 0.9 | 20.2 | −20.1 | −0.1 |
| pH 6.5, Histidine | Pass | 0.01 | 97 | 1.3 | −1.3 | 0.0 | 20.3 | −17.6 | −2.7 |

^{a}Reported as a change in purity relative to the starting material. The starting material (no incubation) contains ≥98.8% native peak by SE-UPLC and ≥60.3% main peak by CEX-UPLC in all formulations.
CEX, cation exchange;
DS, drug substance;
HMW, high molecular weight;
LMW, low molecular weight;
OD, optical density;
RP, reversed-phase;
SE, size-exclusion;
UPLC, ultra performance liquid chromatography

TABLE 3
Effect of Thermal Stabilizer and Surfactants on the Thermal
Stability of 5 mg/mL mAb1 Incubated at 45° C. for 28 Days

| | |
|---|---|
| Formulation | 5 mg/mL mAb1, 10 mM histidine, pH 6.0 |
| Fill Volume | 0.4 mL |
| Container/Closure | 2 mL Type 1 borosilicate glass vial with a FluroTec ®-coated 4432/50 butyl rubber stopper |

| | | | | % Total | | | | Change in | | |
|---|---|---|---|---|---|---|---|---|---|---|
| | | Turbidity | | Protein | Change in Purity | | | Charge Variants | | |
| | | (Increase | | Recovered | by SE-UPLC^{a} | | | by CEX-UPLC^{a} | | |
| Co-Solvent/ | Color and | in OD at | | by RP- | % | % | % | % | % | % |
| Surfactant | Appearance^{a} | 405 nm) | pH | UPLC | HMW | Native | LMW | Acidic | Main | Basic |
| No Co- | Pass | 0.00 | 6.1 | 95 | 0.7 | −0.8 | 0.1 | 15.0 | −13.4 | −1.6 |
| solvent/ | | | | | | | | | | |
| Surfactant | | | | | | | | | | |
| 5% (w/v) | Pass | 0.00 | 6.0 | 97 | 0.5 | −0.6 | 0.1 | 15.8 | −14.9 | −0.8 |
| Sucrose | | | | | | | | | | |
| 10% (w/v) | Pass | 0.00 | 6.1 | 97 | 0.5 | −0.6 | 0.1 | 9.8 | −10.0 | 0.2 |
| Sucrose | | | | | | | | | | |
| 0.05% (w/v) | Pass | 0.01 | 6.0 | 97 | 0.5 | −0.5 | 0.0 | 14.5 | −13.1 | −1.4 |
| Polysorbate | | | | | | | | | | |
| 80 | | | | | | | | | | |
| 0.1% (w/v) | Pass | 0.00 | 6.0 | 98 | 1.0 | −1.5 | 0.5 | 16.9 | −14.6 | −2.3 |
| Polysorbate | | | | | | | | | | |
| 80 | | | | | | | | | | |
| 0.1% (w/v) | Pass | 0.01 | 6.0 | 94 | 0.5 | −0.6 | 0.1 | 13.4 | −12.0 | −1.3 |
| Polysorbate | | | | | | | | | | |
| 20 | | | | | | | | | | |
| 0.05% (w/v) | Pass | 0.02 | 6.1 | 97 | 0.5 | −0.6 | 0.1 | 12.9 | 9.5 | −3.5 |
| Polysorbate | | | | | | | | | | |
| 20, 5% | | | | | | | | | | |
| Sucrose | | | | | | | | | | |
| 0.05% (w/v) | Pass | 0.00 | 6.0 | 97 | 0.5 | −0.6 | 0.1 | 13.6 | −12.1 | −1.5 |
| Polysorbate | | | | | | | | | | |
| 80, 10% | | | | | | | | | | |
| Sucrose | | | | | | | | | | |
| 0.1% (w/v) | Pass | 0.00 | 6.0 | 98 | 0.5 | −0.6 | 0.1 | 17.6 | −15.6 | −2.0 |
| Polysorbate | | | | | | | | | | |
| 20, 10% | | | | | | | | | | |
| Sucrose | | | | | | | | | | |
| 0.1% (w/v) | Pass | 0.00 | 6.0 | 97 | 0.5 | −0.7 | 0.1 | 13.9 | −13.2 | 0.8 |
| Polysorbate | | | | | | | | | | |
| 80, 5% | | | | | | | | | | |
| Sucrose | | | | | | | | | | |
| 0.1% (w/v) | Pass | 0.00 | 6.0 | 97 | 0.5 | −0.6 | 0.1 | 16.6 | −15.1 | −1.5 |
| Polysorbate | | | | | | | | | | |
| 80, 10% | | | | | | | | | | |
| Sucrose | | | | | | | | | | |

^{a}Reported as a change in purity relative to the starting material. The starting material (no incubation) contains ≥98.8% native peak by SE-UPLC and ≥60.3% main peak by CEX-UPLC in all formulations.
CEX, cation exchange;
DS, drug substance;
HMW, high molecular weight;
LMW, low molecular weight;
OD, optical density;
RP, reversed-phase;
SE, size-exclusion;
UPLC, ultra performance liquid chromatography

TABLE 4
Effect of Thermal Stabilizer and Surfactants on the Stability
of 5 mg/mL mAb1 After Agitation (120 Minutes of Vortexing)

| | |
|---|---|
| Formulation | 5 mg/mL mAb1, 10 mM histidine, pH 6.0 |
| Fill Volume | 0.4 mL |
| Container/Closure | 2 mL Type 1 borosilicate glass vial with a FluroTec ®-coated 4432/50 butyl rubber stopper |

| | | | | % Total | | | | Change in | | |
|---|---|---|---|---|---|---|---|---|---|---|
| | | Turbidity | | Protein | Change in Purity | | | Charge Variants | | |
| | | (Increase | | Recovered | by SE-UPLC^{a} | | | by CEX-UPLC^{a} | | |
| Co-Solvent/ | Color and | in OD at | | by RP- | % | % | % | % | % | % |
| Surfactant | Appearance | 405 nm) | pH | UPLC | HMW | Native | LMW | Acidic | Main | Basic |
| No Co- | Pass | 0.02 | 6.0 | 93 | 35.5 | −35.5 | 0.1 | −0.4 | −0.3 | 0.7 |
| solvent/ | | | | | | | | | | |
| Surfactant | | | | | | | | | | |
| 5% (w/v) | Pass | 0.02 | 6.0 | 94 | 40.7 | −40.7 | 0.0 | −1.7 | 0.9 | 0.8 |
| Sucrose | | | | | | | | | | |
| 10% (w/v) | Pass | 0.02 | 6.0 | 94 | 48.7 | −48.7 | 0.1 | −1.6 | 1.2 | 0.5 |
| Sucrose | | | | | | | | | | |
| 0.05% (w/v) | Pass | 0.00 | 6.0 | 99 | 0.0 | 0.1 | −0.1 | 1.2 | −1.0 | −0.2 |
| Polysorbate | | | | | | | | | | |
| 80 | | | | | | | | | | |
| 0.1% (w/v) | Pass | 0.00 | 6.0 | 101 | 0.0 | 0.0 | 0.0 | 1.0 | −0.5 | −0.5 |
| Polysorbate | | | | | | | | | | |
| 80 | | | | | | | | | | |
| 0.1% (w/v) | Pass | 0.00 | 6.0 | 99 | 0.0 | 0.0 | 0.0 | 1.3 | −0.6 | −0.6 |
| Polysorbate | | | | | | | | | | |
| 20 | | | | | | | | | | |
| 0.05% (w/v) | Pass | 0.00 | 6.0 | 98 | 0.0 | 0.0 | 0.0 | 2.6 | −2.4 | −0.2 |
| Polysorbate | | | | | | | | | | |
| 20, 5% | | | | | | | | | | |
| Sucrose | | | | | | | | | | |
| 0.1% (w/v) | Pass | 0.00 | 6.0 | 98 | 0.0 | 0.0 | 0.0 | 2.1 | −1.7 | −0.3 |
| Polysorbate | | | | | | | | | | |
| 20, 10% | | | | | | | | | | |
| Sucrose | | | | | | | | | | |
| 0.05% (w/v) | Pass | 0.01 | 6.0 | 97 | 0.0 | 0.0 | 0.0 | 1.3 | −1.1 | 0.2 |
| Polysorbate | | | | | | | | | | |
| 80, 10% | | | | | | | | | | |
| Sucrose | | | | | | | | | | |
| 0.1% (w/v) | Pass | 0.00 | 6.0 | 98 | 0.0 | 0.0 | 0.0 | 1.1 | −0.8 | −0.3 |
| Polysorbate | | | | | | | | | | |
| 80, 5% | | | | | | | | | | |
| Sucrose | | | | | | | | | | |
| 0.1% (w/v) | Pass | 0.00 | 6.0 | 98 | 0.0 | −0.1 | 0.1 | 2.1 | −1.9 | −0.2 |
| Polysorbate | | | | | | | | | | |
| 80, 10% | | | | | | | | | | |
| Sucrose | | | | | | | | | | |

^{a}Reported as a change in purity relative to the starting material. The starting material (no incubation) contains ≥98.8% native peak by SE-UPLC and ≥60.3% main peak by CEX-UPLC in all formulations.
CEX, cation exchange;
DS, drug substance;
HMW, high molecular weight;
LMW, low molecular weight;
OD, optical density;
RP, reversed-phase;
SE, size-exclusion;
UPLC, ultra performance liquid chromatography

TABLE 5
Effect of Thermal Stabilizer and Surfactants on the Stability
of 5 mg/mL mAb1 After 8 Freezing and Thawing Cycles

| | |
|---|---|
| Formulation | 5 mg/mL mAb1, 10 mM histidine, pH 6.0 |
| Fill Volume | 0.4 mL |
| Container/Closure | 2 mL Type 1 borosilicate glass vial with a FluroTec ®-coated 4432/50 butyl rubber stopper |

| | | | | % Total | | | | Change in | | |
|---|---|---|---|---|---|---|---|---|---|---|
| | | Turbidity | | Protein | Change in Purity | | | Charge Variants | | |
| | | (Increase | | Recovered | by SE-UPLC^{a} | | | by CEX-UPLC^{a} | | |
| Co-Solvent/ | Color and | in OD at | | by RP- | % | % | % | % | % | % |
| Surfactant | Appearance^{a} | 405 nm) | pH | UPLC | HMW | Native | LMW | Acidic | Main | Basic |
| No Co- | Pass | 0.00 | 6.0 | 98 | 0.0 | 0.0 | 0.0 | −0.4 | 0.6 | −0.3 |
| solvent/ | | | | | | | | | | |
| Surfactant | | | | | | | | | | |
| 5% (w/v) | Pass | 0.00 | 6.0 | 94 | 0.3 | −0.3 | 0.0 | 1.6 | −1.4 | −0.2 |
| Sucrose | | | | | | | | | | |
| 10% (w/v) | Pass | 0.00 | 6.0 | 96 | 0.5 | −0.5 | 0.0 | 0.1 | 0.0 | 0.0 |
| Sucrose | | | | | | | | | | |
| 0.05% (w/v) | Pass | 0.00 | 6.0 | 96 | 0.0 | 0.1 | −0.1 | 0.0 | 0.0 | 0.0 |
| Polysorbate | | | | | | | | | | |
| 80 | | | | | | | | | | |
| 0.1% (w/v) | Pass | 0.00 | 6.0 | 99 | 0.0 | 0.0 | 0.0 | 0.3 | 0.0 | −0.3 |
| Polysorbate | | | | | | | | | | |
| 80 | | | | | | | | | | |
| 0.1% (w/v) | Pass | 0.00 | 6.0 | 97 | 0.0 | 0.0 | 0.0 | −0.3 | 0.2 | 0.1 |
| Polysorbate | | | | | | | | | | |
| 20 | | | | | | | | | | |
| 0.05% (w/v) | Pass | 0.00 | 6.0 | 96 | 0.0 | 0.0 | 0.0 | 2.0 | −2.1 | 0.1 |
| Polysorbate | | | | | | | | | | |
| 20, 5% | | | | | | | | | | |
| Sucrose | | | | | | | | | | |
| 0.1% (w/v) | Pass | 0.00 | 6.0 | 97 | 0.0 | 0.0 | 0.0 | 2.9 | −2.7 | −0.2 |
| Polysorbate | | | | | | | | | | |
| 20, 10% | | | | | | | | | | |
| Sucrose | | | | | | | | | | |
| 0.05% (w/v) | Pass | 0.00 | 6.0 | 96 | 0.0 | −0.1 | 0.0 | 1.5 | −1.2 | −0.3 |
| Polysorbate | | | | | | | | | | |
| 80, 10% | | | | | | | | | | |
| Sucrose | | | | | | | | | | |
| 0.1% (w/v) | Pass | 0.00 | 6.0 | 95 | 0.0 | 0.0 | 0.0 | 0.1 | −0.2 | 0.1 |
| Polysorbate | | | | | | | | | | |
| 80, 5% | | | | | | | | | | |
| Sucrose | | | | | | | | | | |
| 0.1% (w/v) | Pass | 0.00 | 6.0 | 97 | 0.0 | 0.0 | 0.0 | 0.7 | −0.5 | −0.2 |
| Polysorbate | | | | | | | | | | |
| 80, 10% | | | | | | | | | | |
| Sucrose | | | | | | | | | | |

^{a}Reported as a change in purity relative to the starting material. The starting material (no incubation) contains ≥98.8% native peak by SE-UPLC and ≥60.3% main peak by CEX-UPLC in all five formulations.
CEX, cation exchange;
DS, drug substance;
HMW, high molecular weight;
LMW, low molecular weight;
OD, optical density;
RP, reversed-phase;
SE, size-exclusion;
UPLC, ultra-performance liquid chromatography

### Summary of Selected Formulation Components

mAb exhibited maximal stability when formulated in the presence of 10+2 mM histidine at pH 6.0+0.3, 0.1%+0.05% (w/v) polysorbate 80, and 10%+2% (w/v) sucrose.

### Example 3: Forced Degradation Study of Anti-BCMA×Anti-CD3 Bispecific Antibody Formulations

This study was conducted to examine the impact of commonly encountered stress conditions upon mAb1. The study was designed to examine the impact of pH, temperature, light exposure, oxidation, agitation, and freeze-thaw cycling. These factors were studied in the ranges and conditions described in Table 6, below. Degradation in this study was examined at both 2 and 100 mg/ml mAb1.

TABLE 6
Stress Conditions and Sample Compositions

| Factor | Stressed Condition | Sample Compositions |
|---|---|---|
| Temperature | 40° C. 75% RH, 14, 28 d | 2 and 100 mg/mL mAb1 |
| pH^{a} | 5.0-8.0 | 10 mM histidine, pH 5.0 |
| | | 10 mM histidine, pH 6.0 |
| | | 10 mM histidine, pH 7.0 |
| | | 10 mM phosphate, pH 8.0 |
| Agitation | 48 hr at 250 RPM | 2 and 100 mg/mL mAb1 |
| Freeze-thaw | 8x freeze thaw at −30° C. | 10 mM histidine, pH 6.0 |
| Oxidation | 0 ppm H₂O₂ at 25° C., 24 hr | |
| | 1 ppm H₂O₂ at 25° C., 24 hr | |
| | 10 ppm H₂O₂ at 25° C., 24 hr | |
| Photostability | ½ ICH Q1B cool white (600,000 lux hours | |
| | at 400-800 nm) 3000 lux, 8 days | |

^{a}Samples at pH 5.0, 6.0, and 7.0 prepared with 10 mM histidine buffer; pH 8.0 prepared with 10 mM phosphate buffer

As described in Table 6, pH stress samples were prepared at pH values of 5.0, 6.0, 7.0, and 8.0. pH stress samples were prepared in 10 mM histidine buffer for pH 5.0, 6.0, and 7.0, and 10 mM phosphate buffer for pH 8.0. Agitation, freeze-thaw, oxidation, and photostability samples were prepared in 10 mM histidine buffer at pH 6.0. All samples prepared in this study were filled in 2 mL borosilicate glass vials.

The following protocols were executed for each condition:
Temperature and pH: Samples were prepared in borosilicate glass vials at pH values of 5.0, 6.0, 7.0, and 8.0. Samples were incubated at 40° C. 75% RH for a total of 28 days, with timepoints taken as specified.
Agitation: Samples were secured in an orbital shaker in a sideways orientation at 250 RPM and ambient temperature and light conditions. Samples were collected after 48 hours.
Freeze-thaw: Samples were frozen at −30° C. and thawed at ambient temperature a total of 8 times. Samples were frozen at −30° C. for a minimum of 4 hours.
Oxidation: H₂O₂ samples were spiked with 0, 1, and 10 ppm H₂O₂ from a 0.1% H₂O₂ stock solution and then incubated at 25° C. for a total of 24 hours.
Photostability: Photostability samples were placed in a cool white light chamber at 3200 lux (400-800 nm) for approximately 187 hours, comprising approximately 600 klux-hours or 0.5× the ICH condition for cool white light. Region 1 includes peaks that elute before the main charge variant peak. These are relatively acidic compared to the main charge variant peak. Region 2 is the main charge variant peak. Region 3 includes peaks that elute after the main charge variant peak. These are relatively basic compared to the main charge variant peak.

### pH and Temperature Stress

Table 7 and Table 8 provide pH and temperature stress data for 2 and 100 mg/mL samples stored at 40° C., 75% RH for up to 28 days. mAb1 exhibited significant degradation when solution pH was greater or equal to 7.0 at both low and high protein concentrations, as evidenced by visual failures at pH 7.0 and 8.0 conditions. Although both 2 and 100 mg/mL mAb1 were susceptible to degradation at high pH, 100 mg/mL appeared more susceptible to HMW generation than 2 mg/mL. Region 1 generation was also evident in high pH samples and appeared equivalent at 2 and 100 mg/mL. Based on these data, subvisible particulate generation at high concentration was also a degradation mechanism at high pH values, as elevated 2-10, ≥10, and ≥25 μm particulate concentrations were noted after 28 days at 40° C., 75% RH at all pH values, but with increasing concentration at higher pH values. At low protein concentration, particulate generation ≥10 and ≥25 μm was noted at higher pH values, but pH 6 samples exhibited the highest number of 2-10 μm particles after 28 days at 40° C., 75% RH.

Based on these data, predominant mechanisms of degradation at pH≥7.0 were generation of HMW and acidic species as determined by SE-UPLC, and CEX-UPLC analysis, with LCMS data indicating increases in deamidation, oxidation, and N-terminal pyroglutamate formation as well as decrease in lysine glycation levels at high pH. 2 mg/mL mAb1 at pH 8.0 also exhibited binding assay failure with a potency of 19%. All other samples at pH 7 and 8 exhibited a decrease in binding activity but were within the potency acceptance criteria of 50-150%.

### Photoexposure Stress

mAb1 stability when exposed to up to 600 klux-hr of cool white light at 25° C. is shown in Table 9. These data suggest mAb1 susceptibility to cool white light exposure at high concentrations, as evidenced by increases in % HMW and % Region 3 seen at 100 mg/mL after 600 klux-hr light exposure. These data and LCMS data (Table 9) also suggest that oxidation is a degradation pathway for mAb1 upon exposure to ambient light. Both high and low protein concentrations exhibited lower potency than control samples but met potency criteria of 50-150%.

### Oxidation Stress

mAb1 stability when exposed to 1 and 10 ppm H₂O₂ is shown in Table 10. Based upon these data, there was minimal susceptibility of mAb1 to 1 or 10 ppm H₂O₂. The only charged species change observed was an increase in Region 3 (basic species) at 10 ppm H₂O₂ in 2 mg/mL mAb1, but Table 13 notes an increase in oxidative species at 10 ppm H₂O₂ in both 2 and 100 mg/mL samples. All oxidation stress data were within the potency acceptance criteria of 50-150%.

### Freeze-Thaw Stress

mAb1 stability after 8 freeze-thaw cycles from −30° C. is shown in Table 11. Based on these data, susceptibility to freeze-thaw cycling was observed at both 2 and 100 mg/mL protein, with major degradation mechanisms of HMW and subvisible particulate generation, particularly at 100 mg/mL mAb1. These data suggested necessity of a thermostabilizer to prevent HMW and subvisible particulate-based degradation. All mAb freeze-thaw data were within the potency acceptance criteria of 50-150%.

### Agitation Stress

mAb1 stability after 48 hours at 250 RPM is shown in Table 12. Susceptibility to agitation was noted at 2 and 100 mg/mL protein. Based upon these data, major degradation pathways from agitation stress were generation of HMW species and particulates, both subvisible and visible. Generation of HMW species was noted in both concentrations but was more pronounced at 2 mg/mL mAb1. Further, significant generation of subvisible and visible particulates was observed after agitation, and a loss in protein concentration was observed in 2 mg/mL mAb1. All mAb1 agitation data were within the potency acceptance criteria of 50-150%.

TABLE 7
Temperature and pH Stress, 2 mg/ml

| | |
|---|---|
| Formulation | 2.0 mg/mL mAb1 in 10 mM histidine buffer at described pH |
| Storage | 6R type I borosilicate glass vial with 20 mm rubber stopper |
| Container | |
| Storage | 40° C., 75% RH, Upright |
| Condition | |

| | | | Assay | | | | | | | | | | | |
|---|---|---|---|---|---|---|---|---|---|---|---|---|---|---|
| | | | Length of Storage (days) | | | | | | | | | | | |
| | | | 0 | 14 | 28 | 0 | 14 | 28 | 0 | 14 | 28 | 0 | 14 | 28 |
| Target Sample pH | | | 5.0 | | | 6.0 | | | 7.0 | | | 8.0 | | |
| Visual Inspection | | | Pass | Pass | Pass | Pass | Pass | Pass | Pass | Pass | Pass | Pass | Pass | Pass |
| Δ OD at 405 nm | | | 0.00 | 0.00 | 0.00 | 0.00 | 0.00 | 0.00 | 0.00 | 0.00 | 0.01 | 0.00 | 0.00 | 0.01 |
| pH | | | 5.1 | 5.2 | 5.1 | 6.0 | 6.1 | 6.1 | 7.0 | 7.0 | 6.9 | 7.9 | 8.0 | 7.9 |
| Total Protein (mg/mL) | | | 1.9 | 2.0 | 2.0 | 1.9 | 1.9 | 1.9 | 1.5 | 1.5 | 1.6 | 1.9 | 1.9 | 1.9 |
| Potency by ELISA (%) | | | NR | NR | 54 | 83 | NR | 71 | NR | NR | 69 | NR | NR | 19 |
| Purity by | % HMW | | 0.5 | 0.4 | 0.6 | 0.5 | 0.5 | 0.6 | 0.5 | 0.6 | 0.8 | 1.2 | 8.6 | 11.7 |
| SE-UPLC | % Main | | 99.6 | 99.5 | 99.3 | 99.5 | 99.5 | 99.3 | 99.5 | 99.4 | 99.1 | 98.8 | 91.1 | 88.0 |
| | % LMW | | 0.0 | 0.0 | 0.2 | 0.0 | 0.1 | 0.1 | 0.0 | 0.0 | 0.1 | 0.0 | 0.3 | 0.4 |
| Charge | % Region 1 | | 34.0 | 31.9 | 29.0 | 32.8 | 37.3 | 40.4 | 39.0 | 47.1 | 54.6 | 35.9 | 79.5 | 84.1 |
| Variant | % Region 2 | | 53.2 | 51.4 | 56.1 | 54.0 | 48.7 | 45.6 | 50.3 | 43.0 | 36.4 | 51.3 | 20.5 | 15.9 |
| Analysis | % Region 3 | | 12.8 | 16.7 | 14.9 | 13.3 | 14.0 | 14.0 | 10.8 | 9.9 | 9.0 | 12.8 | 0.0 | 0.0 |
| by CEX | | | | | | | | | | | | | | |
| Particulate | 2-10 | μm | 26 | NR | 534 | 444 | NR | 19799 | 3015 | NR | 9816 | 1937 | NR | 2020 |
| Matter by | ≥10 | μm | 13 | NR | 146 | 38 | NR | 211 | 596 | NR | 509 | 580 | NR | 471 |
| MFI ™ | ≥25 | μm | 7 | NR | 38 | 2 | NR | 63 | 147 | NR | 186 | 131 | NR | 83 |
| (particles/mL) | | | | | | | | | | | | | | |

TABLE 8
Temperature and pH Stress, 100 mg/mL

| | |
|---|---|
| Formulation | 100.0 mg/mL mAb1 in 10 mM histidine buffer at described pH |
| Storage Container | 6R type I borosilicate glass vial with 20 mm rubber stopper |
| Storage Condition | 40° C., 75% RH, Upright |

| | | | Assay | | | | | | | | | | | |
|---|---|---|---|---|---|---|---|---|---|---|---|---|---|---|
| | | | Length of Storage (days) | | | | | | | | | | | |
| | | | 0 | 14^{a} | 28 | 0 | 14 | 28 | 0 | 14 | 28 | 0 | 14 | 28 |
| Target Sample pH | | | 5.0 | | | 6.0 | | | 7.0 | | | 8.0 | | |
| Visual Inspection | | | Pass | NA | Pass | Pass | Pass | Pass | Pass | Pass | Fail | Fail | Fail | Fail |
| Δ OD at 405 nm | | | 0.00 | NA | 0.01 | 0.00 | 0.01 | 0.01 | 0.00 | 0.04 | 0.08 | 0.00 | 0.03 | 0.09 |
| pH | | | 5.4 | NA | 5.5 | 6.1 | 6.1 | 6.2 | 7.3 | 7.3 | 7.3 | 7.8 | 7.8 | 7.8 |
| Total Protein (mg/mL) | | | 87 | NA | 101 | 88 | 94 | 102 | 97 | 96 | 99 | 89 | 96 | 110 |
| Potency by ELISA (%) | | | NA | NR | 74 | 119 | NA | 90 | NA | NA | 69 | NA | NA | 59 |
| Purity by | % HMW | | 0.8 | NA | 2.7 | 1.0 | 2.2 | 2.9 | 1.3 | 4.5 | 6.5 | 2.0 | 9.8 | 14.4 |
| SE-UPLC | % Main | | 99.2 | NA | 97.2 | 99.1 | 97.8 | 97.0 | 98.7 | 95.4 | 93.5 | 98.0 | 90.0 | 85.5 |
| | % LMW | | 0.0 | NA | 0.1 | 0.0 | 0.1 | 0.0 | 0.0 | 0.1 | 0.1 | 0.0 | 0.2 | 0.1 |
| Charge | % Region 1 | | 33.7 | NA | 34.7 | 34.6 | 35.4 | 40.1 | 34.6 | 45.2 | 53.5 | 35.2 | 61.1 | 75.9 |
| Variant | % Region 2 | | 52.8 | NA | 47.4 | 52.0 | 51.1 | 43.9 | 52.3 | 44.0 | 37.5 | 51.5 | 30.6 | 24.1 |
| Analysis | % Region 3 | | 13.5 | NA | 17.9 | 13.4 | 13.6 | 16.0 | 13.1 | 10.8 | 9.0 | 13.3 | 8.3 | 0.0 |
| by CEX | | | | | | | | | | | | | | |
| Particulate | 2-10 | μm | 1003 | NR | 20371 | 269 | NR | 16879 | 10221 | NR | 69618 | 2372 | NR | 91377 |
| Matter by | ≥10 | μm | 49 | NR | 78 | 16 | NR | 587 | 685 | NR | 4253 | 111 | NR | 2903 |
| MFI ™ | ≥25 | μm | 16 | NR | 0 | 3 | NR | 20 | 52 | NR | 308 | 3 | NR | 324 |
| (particles/mL) | | | | | | | | | | | | | | |

TABLE 9
Photoexposure Stress

| | |
|---|---|
| Formulation | 2.0 & 100.0 mg/mL mAb1 in 10 mM histidine |
| | buffer, pH 6.0 |
| Storage | 6R type I borosilicate glass vial with 20 |
| Container | mm rubber stopper |
| Storage | 25° C. |
| Condition | |

| | | | Assay | | | |
|---|---|---|---|---|---|---|
| | | | Cool White Photoexposure (klux-hr) | | | |
| | | | 0 | 600 | 0 | 600 |
| | | | Target Protein Concentration (mg/mL) | | | |
| | | | 2.0 | | 100.0 | |
| Visual Inspection | | | Pass | Pass | Pass | Pass |
| Δ OD at 405 nm | | | 0.00 | 0.00 | 0.00 | 0.07 |
| pH | | | 6.0 | 6.1 | 6.1 | 6.0 |
| Total Protein (mg/mL) | | | 1.9 | 2.0 | 88 | 93 |
| Potency by ELISA | | | 83 | 60 | 119 | 74 |
| Purity by | % HMW | | 0.5 | 0.9 | 1.0 | 10.4 |
| SE-UPLC | % Main | | 99.5 | 99.1 | 99.1 | 89.6 |
| | % LMW | | 0.0 | 0.0 | 0.0 | 0.0 |
| Charge | % Region 1 | | 32.8 | 40.4 | 34.6 | 41.5 |
| Variant | % Region 2 | | 54.0 | 41.7 | 52.0 | 26.0 |
| Analysis | % Region 3 | | 13.3 | 17.9 | 13.4 | 32.6 |
| by CEX | | | | | | |
| Particulate | 2-10 | μm | 444 | 244 | 269 | 570 |
| Matter by | ≥10 | μm | 38 | 38 | 16 | 10 |
| MFI ™ | ≥25 | μm | 2 | 2 | 3 | 7 |
| (particles/mL) | | | | | | |

TABLE 10
Oxidation Stress

| | |
|---|---|
| Formulation | 2.0 & 100.0 mg/mL mAb1 in 10 mM histidine buffer, pH 6.0 |
| Storage Container | 6R type I borosilicate glass vial with 20 mm rubber stopper |
| Storage Condition | 25° C., 24 hours |

| | | | Assay | | | | | |
|---|---|---|---|---|---|---|---|---|
| | | | H₂O₂ Concentration (ppm) | | | | | |
| | | | 0 | 1 | 10 | 0 | 1 | 10 |
| | | | Target Protein Concentration (mg/mL) | | | | | |
| | | | 2.0 | | | 100.0 | | |
| Visual Inspection | | | Pass | Pass | Pass | Pass | Pass | Pass |
| Δ OD at 405 nm | | | 0.00 | 0.00 | 0.00 | 0.00 | 0.00 | 0.00 |
| pH | | | 6.0 | 6.1 | 6.1 | 6.1 | 6.1 | 6.1 |
| Total Protein (mg/mL) | | | 1.9 | 2.2 | 2.0 | 98.0 | 96.8 | 99.9 |
| Potency by ELISA | | | 83 | 84 | 84 | 119 | 114 | 114 |
| Purity by | % HMW | | 0.5 | 0.5 | 0.5 | 1.2 | 1.2 | 1.2 |
| SE-UPLC | % Main | | 99.5 | 99.5 | 99.5 | 98.8 | 98.8 | 98.8 |
| | % LMW | | 0.0 | 0.0 | 0.0 | 0.0 | 0.0 | 0.0 |
| Charge | % Region 1 | | 33.5 | 33.1 | 28.5 | 31.9 | 33.8 | 31.6 |
| Variant | % Region 2 | | 53.4 | 52.4 | 46.6 | 54.6 | 52.4 | 51.4 |
| Analysis | % Region 3 | | 13.1 | 14.5 | 24.9 | 13.5 | 13.8 | 17.0 |
| by CEX | | | | | | | | |
| Particulate | 2-10 | μm | 188 | 755 | 646 | 2527 | 2159 | 2201 |
| Matter by | ≥10 | μm | 17 | 54 | 75 | 82 | 79 | 43 |
| MFI ™ | ≥25 | μm | 0 | 4 | 10 | 7 | 13 | 3 |
| (particles/mL) | | | | | | | | |

TABLE 11
Freeze-Thaw Stress

| | |
|---|---|
| Formulation | 2.0 mg/mL mAb1 in 10 mM histidine buffer, pH 6.0 |
| Storage Container | 6R type I borosilicate glass vial with 20 mm rubber |
| | stopper |
| Storage Condition | Freeze thaw cycling from −30° C. |

| | | Assay | | | |
|---|---|---|---|---|---|
| | | Freeze-Thaw Cycles from −30° C. | | | |
| | | 0 | 8 | 0 | 8 |
| | | Target Protein | | | |
| | | Concentration (mg/mL) | | | |
| | | 2.0 | | 100.0 | |
| Visual Inspection | | Pass | Pass | Pass | Pass |
| Δ OD at 405 nm | | 0.00 | 0.01 | 0.00 | 0.00 |
| pH | | 6.0 | 6.1 | 6.1 | 6.1 |
| Total Protein | | 1.9 | 1.9 | 88 | 99 |
| Potency by ELISA | | 83 | 130 | 119 | 95 |
| Purity by | % HMW | 0.5 | 0.8 | 1.0 | 3.1 |
| SE-UPLC | % Main | 99.5 | 99.2 | 99.1 | 96.8 |
| | % LMW | 0.0 | 0.0 | 0.0 | 0.1 |
| Charge | % Region 1 | 32.8 | 34.3 | 34.6 | 32.1 |
| Variant | % Region 2 | 54.0 | 52.5 | 52.0 | 54.2 |
| Analysis by | % Region 3 | 13.3 | 13.2 | 13.4 | 13.7 |
| CEX | | | | | |
| Particulate | 2-10 μm | 444 | 24720 | 269 | 188580 |
| Matter by | | | | | |
| MFI ™ | ≥10 μm | 38 | 85 | 16 | 66833 |
| (particles/mL) | ≥25 μm | 2 | 4 | 3 | 20189 |

TABLE 12
Agitation Stress

| Formulation | 2.0 & 100.0 mg/mL mAb1 in 10 mM histidine buffer, | | | | |
|---|---|---|---|---|---|
| | pH 6.0 | | | | |
| Storage Container | 6R type I borosilicate glass vial with 20 mm rubber | | | | |
| | stopper | | | | |
| Storage Condition | 250 RPM at ambient temperature | | | | |
| | | Assay | | | |
| | | 250 RPM (hours) | | | |
| | | 0 | 48 | 0 | 48 |
| | | Target Protein | | | |
| | | Concentration (mg/mL) | | | |
| | | 2.0 | | 100.0 | |
| Visual Inspection | | Pass | Fail | Pass | Pass |
| Δ OD at 405 nm | | 0.00 | 0.44 | 0.00 | 0.01 |
| pH | | 6.0 | 6.1 | 6.1 | 6.1 |
| Total Protein (mg/mL) | | 1.9 | 1.6 | 88 | 91 |
| Potency by ELISA | | 83 | 78 | 119 | 96 |
| Purity by | % HMW | 0.5 | 24.9 | 1.0 | 11.8 |
| SE-UPLC | % Main | 99.5 | 74.9 | 99.1 | 88.2 |
| | % LMW | 0.0 | 0.2 | 0.0 | 0.0 |
| Charge | % Region 1 | 32.8 | 34.8 | 34.6 | 33.3 |
| Variant | % Region 2 | 54.0 | 52.5 | 52.0 | 53.2 |
| Analysis by | % Region 3 | 13.3 | 12.7 | 13.4 | 13.5 |
| CEX | | | | | |
| Particulate | 2-10 μm | 444 | 4095291 | 269 | NA^{a} |
| Matter by | | | | | |
| MFI ™ | ≥10 μm | 38 | 123337 | 16 | NA^{a} |
| (particles/mL) | ≥25 μm | 2 | 2505 | 3 | NA^{a} |

TABLE 13
LC-MS Post-Translational Modification Data Summary

| | |
|---|---|
| Formulation | 2 & 100.0 mg/mL mAb1 in 10 mM histidine buffer at |
| | described pH |

| | | |
|---|---|---|
| Stress Condition | Key Observations | Conclusions |

| pH 5.0-8.0 | Increases | N-terminal | HC/HC* | 1. | Levels of deamidation, |
|---|---|---|---|---|---|
| 40° C., 75% RH | with | Pyroglutamate | | | oxidation, and N-terminal |
| 28 days | increasing | Asparagine | HC Asn319/HC* | | pyroglutamate increase as |
| | pH | Deamidation | Asn318 | | pH increases |
| | | | HC Asn393/HC* | 2. | Levels of lysine glycation |
| | | | Asn392 | | decrease as pH increases |
| | | Methionine | HC Met256/HC* | 3. | Increase in acidic species |
| | | Oxidation | Met255 | | predominantly due to |
| | | | HC Met362/HC* | | deamidation |
| | | | Met361 | 4. | New basic species observed |
| | | | HC Met432/HC* | | at pH 8.0 due to oxidation |
| | | | Met431 | | and N-terminal |
| | Decreases | Lysine | HC* Lys98 | | pyroglutamate formation |
| | with | Glycation | | | |
| | increasing ph | | | | |
| | Identification of HMW Species | | Dimer and | | |
| | | | truncated dimer | | |
| Oxidation | Increased | Methionine | HC Met256/HC* | 1. | No new charge variants |
| 1 & 10 ppm | at 10 ppm | Oxidation | Met255 | | observed at 2 or 100 mg/mL |
| H₂O₂ | H₂O₂ | | HC Met362/HC* | 2. | Methionine oxidation |
| | | | Met361 | | observed in both 2 and 100 |
| | | | HC Met432/HC* | | mg/mL mAb1 at 10 ppm |
| | | | Met431 | | H₂O₂ |
| Photo-Exposure | Increased | Methionine | HC Met256/HC* | 1. | Increases in methionine |
| | in 100 | Oxidation | Met255 | | oxidation observed at 100 |
| | mg/mL | | HC Met362/HC* | | mg/mL after photoexposure |
| | FCP | | Met361 | 2. | No new changes or variants |
| | | | HC Met432 | | observed at 2 mg/mL |
| | | | HC* Met34 | | |
| | | | HC* Met431 | | |

These studies examined mechanisms of mAb1 degradation in a variety of conditions relevant to possible manufacturing, processing, and clinical scenarios. First, mAb1 was susceptible to high pH conditions, and displayed marked increases in HMW and acidic species generation (Tables 7 and 8). Second, mAb1 exhibited sensitivity to cool white light at high concentration (Table 9), minimal sensitivity to hydrogen peroxide oxidation at low and high concentration (Table 10), and sensitivity to freeze-thaw cycling (Table 11) and agitation (Table 12). Finally, LCMS characterization was utilized to characterize changes to mAb1 post-translational modifications, and it was observed that acidic species generation was due predominantly to Asparagine deamidation and basic species generation was due predominantly to Methionine oxidation.

### Example 4: Storage and Stress Stability of Formulations

This Example discloses the storage and stress stability of formulations containing different concentrations of mAb1. The quality attributes and methods used to analyze the stability are listed in Example 1. The results from the long-term storage, accelerated, and stress stability studies, as shown below, indicate that mAb1 will be stable over the course of its clinical use. The mAb1 formulation can withstand short exposures to room temperature without compromising physical or chemical stability. mAb1 formulation will be stored at 2-8° C., and exposure to temperatures greater than 2-8° C. will be limited.

mAb1 formulations containing 2 mg/mL mAb1, 10 mM histidine, pH 6.0, 10% (w/v) sucrose, and 0.1% (w/v) polysorbate 80 were tested for storage and stress stability. The available results from the long-term storage, accelerated, and stress stability studies on the liquid DP indicated that (i) mAb1 2 mg/mL DP can withstand short exposures room temperature and can be frozen and thawed without compromising either the physical or chemical stability of the protein; (ii) mAb1 2 mg/mL DP is stable to interfacial stress and can withstand agitation; (iii) The main degradation pathways identified under accelerated (2500/60% RH) and stressed (4000/75% RH) conditions were the formation of HMW species, LMW species, and acidic charge variants; and (iv) No appreciable changes were detected in any of the monitored mAb1 DP quality attributes after storage at 2-8° C. for 18 months. The results are shown in Tables 14-16.

TABLE 14
Stability of mAb1 Drug Product Stored at 2-8° C.

| | |
|---|---|
| Formulation | 2.0 mg/mL mAb1 in a sterile aqueous buffered solution at pH 6.0 containing 10 mM |
| | histidine, 10% (w/v) sucrose, 0.1% (w/v) polysorbate 80 |
| Storage Container | USP/EP Type 1 borosilicate 6R ISO glass vial; 20 mm West S10-F451 4432/50 GRY B2-40 FluroTec ®- |
| | coated chlorobutyl liquid stopper; West 20 mm aluminum seal with flip-off button |
| Storage Condition | 5° C., Upright |

| | Quality | | | | | | | | | |
|---|---|---|---|---|---|---|---|---|---|---|
| | Acceptance | Length of Storage (months) | | | | | | | | |
| Assay | Criteria | t = 0 | 1 | 3 | 6 | 9 | 12 | 18 | 24 | 36 |
| Physical | Liquid essentially | LEFVP | LEFVP | LEFVP | LEFVP | LEFVP | LEFVP | LEFVP | LEFVP | LEFVP |
| Form/ | free from visible | | | | | | | | | |
| Condition | particulates | | | | | | | | | |
| Clarity | Not more turbid | Not >1 | Not >1 | Not >1 | Not >1 | Not >1 | Not >1 | Not >1 | Not >1 | Not >1 |
| | than reference | | | | | | | | | |
| | suspension IV | | | | | | | | | |
| Color | Not more intensely | Not >BY7 | Not >BY7 | Not >BY7 | Not >BY7 | Not >BY7 | Not >BY7 | Not >BY7 | Not >BY7 | Not >BY7 |
| | colored than | | | | | | | | | |
| | reference solution | | | | | | | | | |
| | BY2 | | | | | | | | | |
| pH | 5.7 to 6.3 | 6.1 | 6.1 | 6.1 | 6.1 | 6.1 | 6.1 | 6.0 | 6.1 | 6.0 |
| Total Protein | 1.8 to 2.2 mg/mL | 2.0 | 2.0 | 2.0 | 2.1 | 2.1 | 2.0 | 2.0 | 2.0 | 2.1 |
| Potency by | Report for | 109 | NR | NR | 124 | NR | 112 | NR | 88 | 79 |
| Bioassay | information | | | | | | | | | |
| | 50%-150% of | | | | | | | | | |
| | reference standard | | | | | | | | | |
| Potency by | Report for | 94 | NR | NR | 105 | NR | 75 | NR | 96 | 81 |
| ELISA | information | | | | | | | | | |
| | 50%-150% of | | | | | | | | | |
| | reference standard | | | | | | | | | |
| Purity by | a. % Purity ≥85% | 96.3 | 95.3 | 95.5 | 94.9 | 96.3 | 96.0 | 96.1 | 95.7 | 94.9 |
| Reduced | total peak area | | | | | | | | | |
| MCE | b. ≤10% LMW | 0.4 | 0.9 | 0.7 | 1.0 | 0.9 | 0.9 | 0.7 | 0.8 | 1.4 |
| | species | | | | | | | | | |
| | c. ≤15% NGHC | 2.9 | 3.3 | 3.2 | 3.7 | 2.5 | 2.7 | 2.6 | 3.1 | 3.2 |
| | species | | | | | | | | | |
| Purity by | a. MP purity ≥85% | 97.5 | 97.0 | 97.7 | 97.7 | 97.7 | 97.8 | 97.6 | 97.2 | 96.5 |
| Non-reduced | total peak area | | | | | | | | | |
| MCE | b. ≤10% LMW | 2.5 | 2.7 | 2.0 | 2.1 | 2.1 | 2.0 | 2.1 | 2.5 | 3.2 |
| | species | | | | | | | | | |
| | c. ≤5% HMW | 0.0 | 0.3 | 0.3 | 0.3 | 0.2 | 0.2 | 0.3 | 0.3 | 0.3 |
| | species | | | | | | | | | |
| Purity by | a. Purity ≥90% | 99.4 | 99.1 | 99.0 | 99.2 | 99.2 | 99.2 | 99.2 | 98.8 | 98.8 |
| SE-UPLC | total peak area | | | | | | | | | |
| | b. ≤5% LMW | 0.0 | 0.0 | 0.0 | 0.0 | 0.0 | 0.0 | 0.0 | 0.0 | 0.1 |
| | species | | | | | | | | | |
| | c. ≤7% HMW | 0.6 | 0.8 | 1.0 | 0.8 | 0.8 | 0.8 | 0.8 | 1.2 | 1.2 |
| | species | | | | | | | | | |
| Charge | a. ≤75% Region 1 | 41.4 | 41.2 | 39.8 | 39.5 | 41.0 | 40.0 | 39.1 | 39.2 | 41.8 |
| Variant | b. ≥25% Region 2 | 49.5 | 49.9 | 50.8 | 51.7 | 49.8 | 50.6 | 51.8 | 50.9 | 48.7 |
| Analysis by | c. ≤25% Region 3 | 9.2 | 8.9 | 9.4 | 8.8 | 9.2 | 9.4 | 9.1 | 9.9 | 9.5 |
| iCIEF | | | | | | | | | | |
| Charge | a. Report % | 37.6 | NR | NR | 35.0 | NR | 35.4 | NR | 36.0 | 37.1 |
| Variant | Region 1 | | | | | | | | | |
| Analysis by | b. Report % | 52.8 | NR | NR | 53.4 | NR | 53.1 | NR | 52.4 | 54.5 |
| CEX | Region 2 | | | | | | | | | |
| | c. Report % | 9.6 | NR | NR | 11.7 | NR | 11.5 | NR | 11.6 | 8.4 |
| | Region 3 | | | | | | | | | |
| Particulate | ≥10 μm: ≤6000 | 4 | NR | 74 | 124 | NR | 158 | NR | 204 | 337 |
| Matter by | (particles/ | | | | | | | | | |
| HIAC | container) | | | | | | | | | |
| (light | ≥25 μm: ≤600 | 0 | NR | 0 | 0 | NR | 0 | NR | 0 | 3 |
| obscuration) | (particles/ | | | | | | | | | |
| | container) | | | | | | | | | |
| Particulate | 2 μm ≤ x < 10 μm | 124 | NR | NR | 1206 | NR | 3305 | NR | 5728 | 6249 |
| Matter by | (particle/mL) | | | | | | | | | |
| MFI ™ | ≥10 μm | 9 | NR | NR | 11 | NR | 90 | NR | 36 | 92 |
| Report | (particle/mL) | | | | | | | | | |
| particle/mL | ≥25 μm | 5 | NR | NR | 0 | NR | 9 | NR | 0 | 5 |
| | (particle/mL) | | | | | | | | | |
| Polysorbate | 0.1 ± 0.05% | 0.11 | NR | NR | 0.11 | NR | 0.11 | NR | 0.11 | 0.10 |
| 80 Content | PS80 | | | | | | | | | |

NR indicates tests not required at the time point, and NA indicates tests required at time points where data has not yet been collected.

TABLE 15
Stability of mAb1 Drug Product Incubated at 25° C./60% RH and 40° C./75% RH

| | |
|---|---|
| Formulation | 2.0 mg/mL mAb1 in a sterile aqueous buffered solution at pH 6.0 |
| | containing 10 mM histidine, 10% (w/v) sucrose, 0.1% (w/v) polysorbate |
| | 80 |
| Storage Container | USP/EP Type 1 borosilicate 6R ISO glass vial; 20 mm West S10-F451 |
| | 4432/50 GRY B2-40 FluroTec ®-coated chlorobutyl liquid stopper; West 20 |
| | mm aluminum seal with flip-off button |

| | | Storage Condition | | | | | | | | |
|---|---|---|---|---|---|---|---|---|---|---|
| | Quality | No | 25° C./60% RH Storage | | | | 40° C./75% RH Storage | | | |
| | Acceptance | Storage | (months) | | | | (months) | | | |
| Assay | Criteria | t = 0 | 0.5 | 1 | 3 | 6 | 0.25 | 0.5 | 1 | 3 |
| Physical | Liquid | LEFVP | LEFVP | LEFVP | LEFVP | LEFVP | LEFVP | LEFVP | LEFVP | LEFVP |
| Form/ | essentially free | | | | | | | | | |
| Condition | from visible | | | | | | | | | |
| | particulates | | | | | | | | | |
| Clarity | Not more | Not >1 | Not >1 | Not >1 | Not >1 | Not >1 | Not >1 | Not >1 | Not >1 | Not >1 |
| | turbid than | | | | | | | | | |
| | reference | | | | | | | | | |
| | suspension IV | | | | | | | | | |
| Color | Not more | Not >BY7 | Not >BY7 | Not >BY7 | Not >BY7 | Not >BY7 | Not >BY7 | Not >BY7 | Not >BY7 | Not >BY7 |
| | intensely | | | | | | | | | |
| | colored than | | | | | | | | | |
| | reference | | | | | | | | | |
| | solution BY2 | | | | | | | | | |
| pH | 5.7 to 6.3 | 6.1 | 6.1 | 6.1 | 6.1 | 6.1 | 6.0 | 6.0 | 6.1 | 6.0 |
| Total | 1.8 to 2.2 | 2.0 | 2.0 | 2.0 | 2.0 | 2.2 | 1.8 | 1.8 | 1.9 | 1.9 |
| Protein | mg/mL | | | | | | | | | |
| Purity by | a. % Purity ≥85% | 96.3 | 95.4 | 95.2 | 95. | 94.2 | 97.0 | 95.6 | 94.4 | 92.4 |
| Reduced | total peak area | | | | | | | | | |
| MCE | b. ≤10% LMW | 0.4 | 1.0 | 1.0 | 1.2 | 1.8 | 0.4 | 1.3 | 2.7 | 3.9 |
| | species | | | | | | | | | |
| | c. ≤15% | 2.9 | 3.2 | 3.3 | 3.0 | 3.3 | 2.1 | 2.6 | 2.4 | 3.0 |
| | NGHC species | | | | | | | | | |
| Purity by | a. MP purity ≥85% | 97.5 | NA | 97.5 | 97.3 | 96.7 | 97.6 | 97.2 | 96.7 | 94.4 |
| Non- | total peak area | | | | | | | | | |
| reduced | b. ≤10% LMW | 2.5 | NA | 2.4 | 2.5 | 3.1 | 2.3 | 2.6 | 3.2 | 5.4 |
| MCE | species | | | | | | | | | |
| | c. % HMW | 0.0 | NA | 0.1 | 0.2 | 0.3 | 0.1 | 0.2 | 0.1 | 0.2 |
| | species | | | | | | | | | |
| Purity by | a. Purity ≥90% | 99.4 | 99.2 | 99.3 | 98.9 | 99.1 | 99.4 | 99.4 | 99.1 | 98.3 |
| SE-UPLC | total peak area | | | | | | | | | |
| | b. ≤5% LMW | 0.0 | 0.0 | 0.0 | 0.2 | 0.1 | 0.1 | 0.1 | 0.2 | 0.4 |
| | species | | | | | | | | | |
| | c. ≤7% HMW | 0.6 | 0.8 | 0.7 | 0.9 | 0.8 | 0.5 | 0.5 | 0.6 | 1.3 |
| | species | | | | | | | | | |
| Charge | a. ≤75% | 41.4 | 40.9 | 40.6 | 42.9 | 49.8 | 42.1 | 44.7 | 52.9 | 80.8 |
| Variant | Region 1 | | | | | | | | | |
| Analysis | b. ≥25% | 49.5 | 49.2 | 49.6 | 47.1 | 41.8 | 48.5 | 46.1 | 40.4 | 13.9 |
| by iCIEF | Region 2 | | | | | | | | | |
| | c. ≤25% | 9.2 | 9.9 | 9.8 | 10.0 | 8.4 | 9.4 | 9.2 | 6.7 | 5.3 |
| | Region 3 | | | | | | | | | |
| Charge | a. % Region 1 | 37.6 | NR | NR | NR | 40.4 | NR | NR | NR | 62.4 |
| Variant | b. % Region 2 | 52.8 | NR | NR | NR | 47.4 | NR | NR | NR | 36.4 |
| Analysis | c. % Region 3 | 9.6 | NR | NR | NR | 12.1 | NR | NR | NR | 1.2 |
| by CEX | | | | | | | | | | |
| Particulate | ≥10 μm: ≤6000 | 4 | NR | NR | 130 | 268 | NR | NR | NR | 114 |
| Matter | particles/ | | | | | | | | | |
| (light | container | | | | | | | | | |
| obscuration) | ≥25 μm: ≤600 | 0 | NR | NR | 0 | 0 | NR | NR | NR | 0 |
| | particles/ | | | | | | | | | |
| | container | | | | | | | | | |
| Particulate | 2 μm ≤ x < 10 μm | 124 | NR | NR | NR | 1560 | NR | NR | NR | 739 |
| Matter | (particle/mL) | | | | | | | | | |
| (MFI ™) | ≥10 μm | 9 | NR | NR | NR | 34 | NR | NR | NR | 13 |
| | (particle/mL) | | | | | | | | | |
| | ≥25 μm | 5 | NR | NR | NR | 0 | NR | NR | NR | 3 |
| | (particle/mL) | | | | | | | | | |
| Polysorbate | Report % | 0.11 | NR | NR | NR | 0.11 | NR | NR | NR | 0.10 |
| 80 Content | PS80 | | | | | | | | | |

NR indicates tests not required at the time point, and NA indicates tests required at time points where data has not yet been collected.
Incubation at 40° C./75% RH was reset due to incubator failure during original time course.

TABLE 16
Stability of mAb1 Drug Product after Agitation and Freeze-Thaw Stress

| Formulation | 2.0 mg/mL mAb1 in a sterile aqueous buffered solution at pH 6.0 containing 10 mM | | | | | |
|---|---|---|---|---|---|---|
| | histidine, 10% (w/v) sucrose, 0.1% (w/v) polysorbate 80 | | | | | |
| Storage | USP/EP Type 1 borosilicate 6R ISO glass vial; 20 mm West S10-F451 4432/50 GRY B2-40 | | | | | |
| Container | FluroTec ®-coated chlorobutyl liquid stopper; West 20 mm aluminum seal with flip-off | | | | | |
| | button | | | | | |
| | | Storage Condition | | | | |
| | | No | 1000 RPM Upright | | Freeze-Thaw | |
| | | Storage | (minutes) | | Cycles | |
| Assay | Quality Acceptance Criteria | t = 0 | 60 | 120 | 2 | 4 |
| Physical Form/ | Liquid essentially free from visible | LEFVP | LEFVP | LEFVP | LEFVP | LEFVP |
| Condition | particulates | | | | | |
| Clarity | Not more turbid than reference | Not >1 | Not >1 | Not >1 | Not >1 | Not >1 |
| | suspension IV | | | | | |
| Color | Not more intensely colored than | Not >BY7 | Not >BY7 | Not >BY7 | Not >BY7 | Not >BY7 |
| | reference solution BY2 | | | | | |
| pH | 5.7 to 6.3 | 6.1 | 6.1 | 6.1 | 6.1 | 6.1 |
| Total Protein | 1.8 to 2.2 mg/mL | 2.0 | 2.0 | 2.0 | 2.0 | 2.0 |
| Purity by | a. % Purity ≥85% total peak area | 96.3 | NR | 96.0 | NR | 95.6 |
| Reduced MCE | b. ≤10% LMW species | 0.4 | NR | 0.7 | NR | 0.9 |
| | c. ≤15% NGHC species | 2.9 | NR | 2.8 | NR | 3.0 |
| Purity by Non- | a. MP purity ≥85% total peak area | 97.5 | NR | 96.9 | NR | 97.5 |
| reduced MCE | b. ≤10% LMW species | 2.5 | NR | 2.7 | NR | 2.2 |
| | c. % HMW species | 0.0 | NR | 0.4 | NR | 0.3 |
| Purity by | a. Purity ≥90% total peak area | 99.4 | 99.1 | 99.1 | 99.0 | 99.0 |
| SE-UPLC | b. ≤5% LMW species | 0.0 | 0.0 | 0.0 | 0.0 | 0.0 |
| | c. ≤7% HMW species | 0.6 | 0.9 | 0.9 | 1.0 | 1.1 |
| Charge Variant | a. ≤75% Region 1 | 41.4 | 41.1 | 40.3 | 40.4 | 39.9 |
| Analysis by | b. ≥25% Region 2 | 49.5 | 49.5 | 50.5 | 50.7 | 50.3 |
| iCIEF | c. ≤ 25% Region 3 | 9.2 | 9.4 | 9.2 | 9.0 | 9.8 |
| Particulate | ≥10 μm: ≤6000 | 4 | NR | 92 | NR | 110 |
| Matter | particles/container | | | | | |
| (light | ≥25 μm: ≤600 | 0 | NR | 2 | NR | 0 |
| obscuration) | particles/container | | | | | |
| | 2 μm ≤ x < 10 μm (particle/mL) | 124 | NR | 1481 | NR | 1753 |
| Particulate | ≥10 μm (particle/mL) | 9 | NR | 13 | NR | 13 |
| Matter (MFI ™) | ≥25 μm (particle/mL) | 5 | NR | 3 | NR | 3 |

NR indicates tests not required at the time point, and NA indicates tests required at time points where data has not yet been collected.

Currently, 36 months of research storage stability data are available for the 10 mg/mL mAb1 formulation. mAb1 formulation was physically and chemically stable when stored at 2-8° C. for at least 36 months (Table 17). SE-UPLC analysis showed a small change in purity. No appreciable change was detected in any of the other monitored attributes.

Results from the analysis of the mAb1 formulation after incubation under accelerated conditions are provided in Table 18. After incubation at 25° C. for 6 months, appreciable formation of HMW species and charge variants (increased relative percentage of acidic species) were detected. However, no appreciable change in the physical or chemical stability was detected after incubation at 25° C. for at least 1 month, indicating that the mAb1 formulation can be exposed to short periods of time at room temperature (less than 1 month). After incubation for 28 days at 45° C., appreciable formation of HMW species and charge variants (increased relative percentage of acidic species) were detected. The results from incubation at accelerated conditions demonstrated that an increase in HMW species and the formation of charge variants were the main degradation pathways for mAb1 formulation.

Results from the mAb1 formulation stress stability studies are provided in Table 19. mAb1 formulation was physically and chemically stable when agitated (vortexed at ambient temperature) for 120 minutes or when subjected to 8 freeze/thaw cycles (freezing at −30° C. and thawing at room temperature).

TABLE 17
Research Stability of 10 mg/mL mAb1 Drug Product Stored at 2-8° C.

| | |
|---|---|
| Formulation | 10 mg/mL mAb1, 10 mM histidine, pH 6.0, 10% (w/v) sucrose, 0.1% (w/v) polysorbate |
| | 80 |
| Fill Volume | 5.5 mL |
| Container/Closure | 10 mL Type 1 borosilicate glass with 20 mm FluroTec ®-coated West S10-F451 4432/50 |
| | gray B2-40 stopper |

| | | | Length of Storage at 2-8° C. (months) | | | | | | | | |
|---|---|---|---|---|---|---|---|---|---|---|---|
| Assay | | | 0 | 1 | 3 | 6 | 9 | 12 | 18 | 24 | 36 |
| Color and Appearance | | | Pass | Pass | Pass | Pass | Pass | Pass | Pass | Pass | Pass |
| Turbidity (Increase in OD at 405 nm) | | | 0.00 | 0.01 | 0.01 | 0.01 | 0.01 | 0.01 | 0.01 | 0.01 | 0.01 |
| pH | | | 6.0 | 6.0 | 6.0 | 6.0 | 6.0 | 6.0 | 6.0 | 6.0 | 6.1 |
| Particulate Analysis | ≥10 | μm | 3 | NR | NR | 40 | NR | 22 | NR | 26 | 5 |
| by Light Obscuration | ≥25 | μm | 1 | NR | NR | 1 | NR | 0 | NR | 1 | 1 |
| (HIAC) (N/mL) | | | | | | | | | | | |
| Particulate Analysis | 2 to 10 | μm | 146 | NR | NR | 2095 | NR | 1559 | NR | 2346 | 4470 |
| by MFI (N/mL) | ≥10 | μm | 0 | NR | NR | 32 | NR | 28 | NR | 8 | 8 |
| | ≥25 | μm | 0 | NR | NR | 5 | NR | 0 | NR | 2 | 2 |

| % Total Protein Recovered by | | 100 | 101 | 105 | 102 | 100 | 103 | 104 | 100 | 100 |
|---|---|---|---|---|---|---|---|---|---|---|
| RP-UPLC | | | | | | | | | | |
| Purity by CE-SDS | Non-reduced; | 97.7 | NR | NR | 96.6 | NR | 98.4 | NR | 98.0 | 96.8 |
| | % main peak | | | | | | | | | |
| | Reduced; | 96.4 | NR | NR | 95.4 | NR | 97.0 | NR | 96.2 | 96.9 |
| | % heavy + | | | | | | | | | |
| | light chain | | | | | | | | | |
| Purity by SE-UPLC | % HMW | 0.9 | 0.9 | 0.9 | 1.0 | 1.1 | 1.1 | 1.2 | 1.3 | 1.4 |
| | % Native | 99.0 | 98.6 | 98.6 | 98.6 | 98.3 | 98.4 | 98.4 | 98.2 | 98.3 |
| | % LMW | 0.1 | 0.6 | 0.5 | 0.5 | 0.6 | 0.4 | 0.4 | 0.6 | 0.4 |
| Charge Variant | % Acidic | 30.8 | 30.1 | 31.6 | 30.9 | 31.5 | 31.5 | 32.1 | 31.5 | 31.1 |
| Analysis by CEX- | % Main | 54.2 | 54.1 | 52.6 | 53.3 | 52.6 | 53.3 | 51.9 | 52.5 | 53.1 |
| UPLC | % Basic | 15.4 | 15.6 | 15.9 | 15.8 | 15.9 | 15.2 | 16.1 | 16.0 | 15.9 |
| Charge Variant | % Acidic | 37.2 | NR | NR | 33.6 | NR | 35.9 | NR | 37.1 | 35.7 |
| Analysis by iCIEF | % Main | 49.7 | NR | NR | 53.0 | NR | 50.7 | NR | 49.0 | 49.9 |
| | % Basic | 13.2 | NR | NR | 13.4 | NR | 13.4 | NR | 13.9 | 14.4 |

| | | | | | | | | | |
|---|---|---|---|---|---|---|---|---|---|
| % Relative Potency by Bioassay | 135 | NR | NR | 114 | NR | 75 | NR | 94 | 98 |

CE-SDS, capillary electrophoresis-sodium dodecyl sulfate;
CEX, cation exchange;
DS, drug substance;
HMW, high molecular weight;
iCIEF, imaged capillary isoelectric focusing;
LMW, low molecular weight;
MFI, Micro-Flow Imaging ™;
NR, not required;
OD, optical density;
RP, reversed-phase;
SE, size-exclusion;
UPLC, ultra performance liquid chromatography

TABLE 18
Research Stability of 10 mg/mL mAb1 Drug Product After Incubation at Accelerated Conditions

| | |
|---|---|
| Formulation | 10 mg/mL mAb1, 10 mM histidine, pH 6.0, 10% (w/v) sucrose, 0.1% (w/v) polysorbate 80 |
| Fill Volume | 5.5 mL |
| Container/Closure | 10 mL Type 1 borosilicate glass with 20 mm FluroTec ® coated West S10-F451 4432/50 gray B2-40 stopper |

| | | | | 25° C./60% RH Storage | | | 45° C. Storage | |
|---|---|---|---|---|---|---|---|---|
| | | | No Storage | (months) | | | (days) | |
| Assay | | | T = 0 | 1 | 3 | 6 | 14 | 28 |
| Color and Appearance | | | Pass | Pass | Pass | Pass | Pass | Pass |
| Turbidity (Increase in OD at 405 nm) | | | 0.00 | 0.01 | 0.01 | 0.01 | 0.00 | 0.01 |
| pH | | | 6.0 | 6.0 | 6.0 | 6.0 | 6.0 | 6.0 |
| Particulate Analysis | ≥10 | μm | 3 | NR | 49 | 20 | NR | 19 |
| by Light | ≥25 | μm | 1 | NR | 1 | 1 | NR | 2 |
| Obscuration (HIAC) | | | | | | | | |
| (N/mL) | | | | | | | | |
| Particulate Analysis | 2 to 10 | μm | 146 | NR | 2699 | 3031 | NR | 744 |
| by MFI (N/mL) | ≥10 | μm | 0 | NR | 11 | 26 | NR | 30 |
| | ≥25 | μm | 0 | NR | 0 | 9 | NR | 3 |

| % Total Protein Recovered by RP-UPLC | | 100 | 103 | 105 | 100 | 100 | 98 |
|---|---|---|---|---|---|---|---|
| Purity by CE-SDS | Non-reduced; | 97.7 | NR | 96.6 | 96.6 | NR | 95.3 |
| | % main peak | | | | | | |
| | Reduced; | 96.4 | NR | 95.4 | 94.3 | NR | 93.2 |
| | % heavy + light | | | | | | |
| | chain | | | | | | |
| Purity by SE-UPLC | % HMW | 0.9 | 0.9 | 1.1 | 1.4 | 1.2 | 1.4 |
| | % Native | 99.0 | 98.5 | 98.4 | 97.2 | 98.3 | 97.7 |
| | % LMW | 0.1 | 0.6 | 0.6 | 0.5 | 0.6 | 0.2 |
| Charge Variant | % Acidic | 30.8 | 29.0 | 32.9 | 35.9 | 33.4 | 41.0 |
| Analysis by CEX- | % Main | 54.2 | 54.4 | 50.9 | 47.9 | 49.6 | 44.3 |
| UPLC | % Basic | 15.4 | 16.6 | 16.3 | 16.2 | 17.0 | 14.8 |
| Charge Variant | % Acidic | 37.2 | NR | 39.1 | 50.8 | NR | 64.9 |
| Analysis by iCIEF | % Main | 49.7 | NR | 47.2 | 34.7 | NR | 35.2 |
| | % Basic | 13.2 | NR | 13.7 | 12.5 | NR | 10.5 |

| | | | | | | |
|---|---|---|---|---|---|---|
| % Relative Potency by Bioassay | 135 | NR | NR | NR | NR | NR |

CE-SDS, capillary electrophoresis-sodium dodecyl sulfate;
CEX, cation exchange;
DS, drug substance;
HMW, high molecular weight;
iCIEF, imaged capillary isoelectric focusing;
LMW, low molecular weight;
MFI, Micro-Flow Imaging ™;
NR, not required;
OD, optical density;
RH, relative humidity;
RP, reversed-phase;
SE, size-exclusion;
UPLC, ultra-performance liquid chromatography

TABLE 19
Research Stability of 10 mg/mL mAb1 Drug Product Under Stress Conditions

| | |
|---|---|
| Formulation | 10 mg/mL mAb1, 10 mM histidine, pH 6.0, 10% (w/v) sucrose, 0.1% (w/v) |
| | polysorbate 80 |
| Fill Volume | 5.5 mL |
| Container/Closure | 10 mL Type 1 borosilicate glass with 20 mm FluroTec ® coated West S10- |
| | F451 4432/50 gray B2-40 stopper |

| | | | No Stress | Agitation (minutes) | | Freeze/Thaw Cycles | |
|---|---|---|---|---|---|---|---|
| Assay | | | T = 0 | 60 | 120 | 4 | 8 |
| Color and Appearance | | | Pass | Pass | Pass | Pass | Pass |
| Turbidity (Increase in OD at 405 nm) | | | 0.00 | 0.00 | 0.00 | 0.01 | 0.01 |
| pH | | | 6.0 | 6.0 | 6.0 | 6.1 | 6.0 |
| Particulate Analysis | ≥10 | μm | 3 | NR | 1 | NR | 16 |
| by Light | ≥25 | μm | 1 | NR | 0 | NR | 1 |
| Obscuration (HIAC) | | | | | | | |
| (N/mL) | | | | | | | |
| Particulate Analysis | 2-10 | μm | 146 | NR | 1223 | NR | 1517 |
| by MFI (N/mL) | ≥10 | μm | 0 | NR | 49 | NR | 13 |
| | ≥25 | μm | 0 | NR | 0 | NR | 5 |

| % Total Protein Recovered by RP-UPLC | | 100 | 97 | 100 | 104 | 111 |
|---|---|---|---|---|---|---|
| Purity by CE-SDS | Non-reduced; | 97.7 | NR | 97.8 | NR | 96.1 |
| | % main peak | | | | | |
| | Reduced; | 96.4 | NR | 96.4 | NR | 96.2 |
| | % heavy + light | | | | | |
| | chain | | | | | |
| Purity by SE-UPLC | % HMW | 0.9 | 0.9 | 0.9 | 0.9 | 0.9 |
| | % Native | 99.0 | 99.0 | 99.0 | 98.6 | 98.6 |
| | % LMW | 0.1 | 0.2 | 0.2 | 0.5 | 0.5 |
| Charge Variant | % Acidic | 30.8 | 29.5 | 29.7 | 31.8 | 31.8 |
| Analysis by CEX- | % Main | 54.2 | 55.0 | 54.6 | 52.8 | 52.8 |
| UPLC | % Basic | 15.4 | 15.5 | 15.8 | 15.4 | 15.4 |
| Charge Variant | % Acidic | 37.2 | NR | 36.3 | NR | 36.0 |
| Analysis by iCIEF | % Main | 49.7 | NR | 50.5 | NR | 50.4 |
| | % Basic | 13.2 | NR | 13.3 | NR | 13.7 |

| | | | | | |
|---|---|---|---|---|---|
| % Relative Potency by Bioassay | 135 | NR | NR | NR | NR |

CE-SDS, capillary electrophoresis-sodium dodecyl sulfate;
CEX, cation exchange;
DS, drug substance;
HMW, high molecular weight;
iCIEF, imaged capillary isoelectric focusing;
LMW, low molecular weight;
MFI, Micro-Flow Imaging ™;
NR, not required;
OD, optical density;
RP, reversed-phase;
SE, size-exclusion;
UPLC, ultra performance liquid chromatography

mAb formulation containing 20 mg/mL mAb1, 10 mM histidine, pH 6.0, 10% (w/v) sucrose, and 0.1% (w/v) polysorbate 80 was tested for stress and storage stability. The available results from the long-term storage, accelerated, and stress stability studies of the liquid DP indicated that (i) mAb1 20 mg/mL DP is stable to interfacial stress and can withstand agitation; (ii) mAb1 20 mg/mL DP can withstand short exposures at room temperature and can be frozen and thawed without compromising either the physical or chemical stability of the protein; (iii) The main degradation pathways identified under accelerated (2500/60% RH) and stressed (4000/75% RH) conditions were the formation of HMW species, LMW species, and acidic charge variants; and (iv) In available data, no appreciable changes were detected in any of the monitored mAb DP quality attributes after storage at 2-8° C. for 9 months. Results are shown below in Tables 20-22.

TABLE 20
Stability of mAb1 Drug Product Stored at 2-8° C.

| | |
|---|---|
| Formulation | 20.0 mg/mL mAb1 in a sterile aqueous buffered solution at pH |
| | 6.0 containing 10 mM histidine, 10% (w/v) sucrose, 0.1% (w/v) polysorbate 80 |
| Storage Container | USP/EP Type 1 borosilicate 20R ISO glass vial; 20 mm West S10-F451 4432/50 GRY B2-40 FluroTec ®- |
| | coated chlorobutyl liquid stopper; West 20 mm aluminum seal with flip-off button |
| Storage Condition | 2-8° C., Upright |

| | Quality | | | | | | | | |
|---|---|---|---|---|---|---|---|---|---|
| | Acceptance | Length of Storage (months) | | | | | | | |
| Assay | Criteria | t = 0 | 1 | 3 | 6 | 9 | 12 | 18 | 24 |
| Physical | Liquid essentially | LEFVP | LEFVP | LEFVP | LEFVP | LEFVP | LEFVP | LEFVP | LEFVP |
| Form/ | free from visible | | | | | | | | |
| Condition | particulates | | | | | | | | |
| Clarity | Not more turbid | Not >II | Not >II | Not >II | Not >II | Not >II | Not >II | Not >II | Not >II |
| | than reference | | | | | | | | |
| | suspension IV | | | | | | | | |
| Color | Not more | Not >BY6 | Not >BY6 | Not >BY6 | Not >BY6 | Not >BY6 | Not >BY6 | Not >BY6 | Not >BY6 |
| | intensely colored | | | | | | | | |
| | than reference | | | | | | | | |
| | solution BY2 | | | | | | | | |
| pH | 5.7 to 6.3 | 6.0 | 6.0 | 6.0 | 6.0 | 6.1 | 6.0 | 6.0 | 6.0 |
| Total Protein | 18 to 22 mg/mL | 19.8 | 19.8 | 19.8 | 19.8 | 19.6 | 19.9 | 19.6 | 20.1 |
| Potency by | 50% to 150% of | 85 | NR | NR | 142 | NR | 95 | 78 | 79 |
| ELISA | reference | | | | | | | | |
| | standard | | | | | | | | |
| Potency by | Report for | 125 | NR | NR | 91 | NR | 96 | 80 | 98 |
| Bioassay | information | | | | | | | | |
| Purity by | a. % Purity ≥85% | 95.6 | 96.3 | 96.0 | 95.9 | 95.4 | 96.4 | 95.7 | 95.7 |
| Reduced | total peak area | | | | | | | | |
| MCE | b. ≤10% LMW | 0.6 | 0.7 | 0.5 | 0.5 | 0.8 | 0.7 | 1.0 | 1.0 |
| | species | | | | | | | | |
| | c. ≤15% NGHC | 3.2 | 2.4 | 3.0 | 3.1 | 3.2 | 2.3 | 2.8 | 2.8 |
| | species | | | | | | | | |
| Purity by Non- | a. MP purity ≥85% | 97.1 | 97.4 | 96.9 | 97.0 | 97.0 | 96.7 | 96.8 | 96.5 |
| reduced MCE | total peak area | | | | | | | | |
| | b. ≤10% LMW | 2.4 | 2.3 | 2.7 | 2.6 | 2.6 | 2.9 | 2.7 | 3.0 |
| | species | | | | | | | | |
| | c. ≤5% HMW | 0.49 | 0.43 | 0.42 | 0.41 | 0.45 | 0.50 | 0.56 | 0.43 |
| | species | | | | | | | | |
| Purity by | a. Purity ≥90% | 99.0 | 99.1 | 99.0 | 99.0 | 99.0 | 99.0 | 98.9 | 98.9 |
| SE-UPLC | total peak area | | | | | | | | |
| | b. ≤5% LMW | 0.0 | 0.0 | 0.0 | 0.0 | 0.1 | 0.0 | 0.1 | 0.0 |
| | species | | | | | | | | |
| | c. ≤7% HMW | 1.0 | 0.9 | 0.9 | 0.9 | 0.9 | 1.0 | 1.0 | 1.1 |
| | species | | | | | | | | |
| Charge | a. ≤75% | 38.7 | 39.1 | 39.5 | 39.2 | 40.2 | 39.3 | 40.0 | 41.2 |
| Variant | Region 1 | | | | | | | | |
| Analysis | b. ≥25% | 52.8 | 52.2 | 52.5 | 52.9 | 51.9 | 51.7 | 50.4 | 50.8 |
| by iCIEF | Region 2 | | | | | | | | |
| | c. ≤25% | 8.5 | 8.7 | 8.1 | 8.0 | 8.0 | 9.0 | 9.6 | 8.0 |
| | Region 3 | | | | | | | | |
| Charge | a. Report % | 36.0 | NR | NR | 35.6 | NR | 35.7 | 36.8 | 36.9 |
| Variant | Region 1 | | | | | | | | |
| Analysis | b. Report % | 53.0 | NR | NR | 53.5 | NR | 52.6 | 52.1 | 52.9 |
| by CEX | Region 2 | | | | | | | | |
| | c. Report % | 11.1 | NR | NR | 10.9 | NR | 11.7 | 11.1 | 10.2 |
| | Region 3 | | | | | | | | |
| Particulate | ≥10 μm: ≤6000 | 56 | NR | 35 | 60 | NR | 77 | 53 | 95 |
| Matter | (particles/ | | | | | | | | |
| by HIAC | container) | | | | | | | | |
| (light | ≥25 μm: ≤600 | 4 | NR | 0 | 0 | NR | 0 | 0 | 0 |
| obscuration) | (particles/ | | | | | | | | |
| | container) | | | | | | | | |
| Particulate | 2 μm ≤ x < 10 μm | 65 | NR | NR | 1658 | NR | 1435 | 1810 | 1810 |
| Matter by | (particle/mL) | | | | | | | | |
| MFI ™ | ≥10 μm | 0 | NR | NR | 36 | NR | 7 | 9 | 5 |
| Report | (particle/mL) | | | | | | | | |
| particle/mL | ≥25 μm | 0 | NR | NR | 7 | NR | 0 | 0 | 0 |
| | (particle/mL) | | | | | | | | |
| Polysorbate | 0.1 ± 0.05% PS80 | 0.11 | NR | NR | 0.10 | NR | 0.10 | 0.10 | 0.10 |
| 80 Content | | | | | | | | | |

NR indicates tests not required at the time point, and NA indicates tests required at time points where data has not yet been collected.

TABLE 21
Stability of mAb1 Drug Product Incubated at 25° C./60% RH and 40° C./75% RH

| | |
|---|---|
| Formulation | 20.0 mg/mL mAb1 in a sterile aqueous buffered solution at pH 6.0 containing 10 mM |
| | histidine, 10% (w/v) sucrose, 0.1% (w/v) polysorbate 80 |
| Storage Container | USP/EP Type 1 borosilicate 20R ISO glass vial; 20 mm West S10-F451 4432/50 GRY |
| | B2-40 FluroTec ®-coated chlorobutyl liquid stopper; West 20 mm aluminum seal with |
| | flip-off button |

| | | Storage Condition | | | | | | | | |
|---|---|---|---|---|---|---|---|---|---|---|
| | Quality | No | 25° C./60% RH Storage | | | | 40° C./75% Rh Storage | | | |
| | Acceptance | Storage | (months) | | | | (months) | | | |
| Assay | Criteria | t = 0 | 0.5 | 1 | 3 | 6 | 0.25 | 0.5 | 1 | 3 |
| Physical | Liquid | LEFVP | LEFVP | LEFVP | LEFVP | LEFVP | LEFVP | LEFVP | LEFVP | LEFVP |
| Form/ | essentially free | | | | | | | | | |
| Condition | from visible | | | | | | | | | |
| | particulates | | | | | | | | | |
| Clarity | Not more turbid | Not >II | Not >II | Not >II | Not >II | Not >II | Not >II | Not >II | Not >II | Not >II |
| | than reference | | | | | | | | | |
| | suspension IV | | | | | | | | | |
| Color | Not more | Not >BY6 | Not >BY6 | Not >BY6 | Not >BY6 | Not >BY6 | Not >BY6 | Not >BY6 | Not >BY6 | Not >BY6 |
| | intensely | | | | | | | | | |
| | colored than | | | | | | | | | |
| | reference | | | | | | | | | |
| | solution BY2 | | | | | | | | | |
| pH | 5.7 to 6.3 | 6.0 | 6.1 | 6.0 | 6.0 | 6.0 | 6.1 | 6.1 | 6.0 | 6.0 |
| Total Protein | 18 to 22 mg/mL | 19.8 | 19.8 | 19.8 | 19.8 | 19.8 | 19.8 | 19.8 | 19.8 | 19.8 |
| Purity by | a. % Purity ≥85% | 95.6 | 95.0 | 94.3 | 95.1 | 94.5 | 95.0 | 94.7 | 94.2 | 92.0 |
| Reduced | total peak area | | | | | | | | | |
| MCE | b. ≤10% LMW | 0.6 | 0.9 | 1.1 | 1.1 | 1.4 | 0.9 | 1.6 | 2.0 | 4.1 |
| | species | | | | | | | | | |
| | c. ≤15% NGHC | 3.2 | 3.6 | 4.0 | 3.2 | 3.7 | 3.4 | 3.2 | 3.1 | 3.0 |
| | species | | | | | | | | | |
| Purity by | a. MP purity ≥85% | 97.1 | 97.1 | 97.3 | 96.5 | 95.9 | 96.9 | 96.7 | 96.2 | 93.3 |
| Non- | total peak area | | | | | | | | | |
| reduced | b. ≤10% LMW | 2.4 | 2.5 | 2.4 | 3.0 | 3.5 | 2.6 | 2.7 | 3.3 | 5.5 |
| MCE | species | | | | | | | | | |
| | c. % HMW | 0.5 | 0.4 | 0.3 | 0.5 | 0.6 | 0.5 | 0.6 | 0.5 | 1.2 |
| | species | | | | | | | | | |
| Purity by | a. Purity ≥90% | 99.0 | 98.9 | 98.9 | 98.9 | 98.6 | 98.8 | 98.7 | 98.5 | 97.4 |
| SE-UPLC | total peak area | | | | | | | | | |
| | b. ≤5% LMW | 0.0 | 0.1 | 0.2 | 0.1 | 0.1 | 0.2 | 0.1 | 0.2 | 0.3 |
| | species | | | | | | | | | |
| | c. ≤7% HMW | 1.0 | 0.9 | 1.0 | 1.1 | 1.3 | 1.0 | 1.1 | 1.3 | 2.3 |
| | species | | | | | | | | | |
| Charge | a. ≤75% | 38.7 | 39.6 | 39.4 | 41.6 | 44.3 | 39.8 | 43.5 | 50.4 | 83.5 |
| Variant | Region 1 | | | | | | | | | |
| Analysis | b. ≥25% | 52.8 | 51.9 | 51.5 | 49.9 | 47.4 | 51.2 | 47.3 | 39.6 | 12.2 |
| by iCIEF | Region 2 | | | | | | | | | |
| | c. ≤25% | 8.5 | 8.5 | 9.1 | 8.5 | 8.3 | 8.9 | 9.2 | 10.0 | 4.3 |
| | Region 3 | | | | | | | | | |
| Charge | a. % Region 1 | 36.0 | NR | NR | NR | 39.9 | NR | NR | NR | 55.6 |
| Variant | b. % Region 2 | 53.0 | NR | NR | NR | 49.9 | NR | NR | NR | 42.5 |
| Analysis | c. % Region 3 | 11.1 | NR | NR | NR | 10.2 | NR | NR | NR | 2.0 |
| by CEX | | | | | | | | | | |
| Particulate | ≥10 μm: ≤6000 | 56 | NR | NR | 186 | 88 | NR | NR | NR | 53 |
| Matter | particles/ | | | | | | | | | |
| (light | container | | | | | | | | | |
| obscuration) | ≥25 μm: ≤600 | 4 | NR | NR | 0 | 0 | NR | NR | NR | 4 |
| | particles/ | | | | | | | | | |
| | container | | | | | | | | | |
| Particulate | 2 μm ≤ x < 10 μm | 65 | NR | NR | NR | 1404 | NR | NR | NR | 503 |
| Matter | (particle/mL) | | | | | | | | | |
| (MFI ™) | ≥10 μm | 0 | NR | NR | NR | 5 | NR | NR | NR | 13 |
| | (particle/mL) | | | | | | | | | |
| | ≥25 μm | 0 | NR | NR | NR | 0 | NR | NR | NR | 0 |
| | (particle/mL) | | | | | | | | | |
| Polysorbate | Report % PS80 | 0.11 | NR | NR | NR | 0.10 | NR | NR | NR | 0.11 |
| 80 Content | | | | | | | | | | |

NR indicates tests not required at the time point, and NA indicates tests required at time points where data has not yet been collected.

TABLE 22
Stability of mAb1 Drug Product after Agitation and Freeze-Thaw Stress

| Formulation | 20.0 mg/mL mAb1 in a sterile aqueous buffered solution at pH 6.0 containing 10 mM | | | | | |
|---|---|---|---|---|---|---|
| | histidine, 10% (w/v) sucrose, 0.1% (w/v) polysorbate 80 | | | | | |
| Storage | USP/EP Type 1 borosilicate 20R ISO glass vial; 20 mm West S10-F451 4432/50 GRY | | | | | |
| Container | B2-40 FluroTec ®-coated chlorobutyl liquid stopper; West 20 mm aluminum seal with | | | | | |
| | flip-off button | | | | | |
| | | Storage Condition | | | | |
| | | | 1000 RPM | 250 RPM | | |
| | | No | Upright | Sideways | Freeze-Thaw | |
| | | Storage | (minutes) | (hours) | Cycles | |
| Assay | Quality Acceptance Criteria | t = 0 | 120 | 48 | 2 | 4 |
| Physical Form/ | Liquid essentially free from | LEFVP | LEFVP | LEFVP | LEFVP | LEFVP |
| Condition | visible particulates | | | | | |
| Clarity | Not more turbid than | Not >II | Not >II | Not >II | Not >II | Not >II |
| | reference suspension IV | | | | | |
| Color | Not more intensely colored | Not >BY6 | Not >BY6 | Not >BY6 | Not >BY6 | Not >BY6 |
| | than reference solution BY2 | | | | | |
| pH | 5.7 to 6.3 | 6.0 | 6.1 | 6.1 | 6.1 | 6.0 |
| Total Protein | 18 to 22 mg/mL | 19.8 | 19.9 | 19.9 | 19.8 | 19.8 |
| Purity by | a. % Purity ≥85% | 95.6 | 95.7 | 96.0 | 95.7 | 95.6 |
| Reduced MCE | total peak area | | | | | |
| | b. ≤10% LMW species | 0.6 | 0.8 | 0.7 | 0.8 | 0.9 |
| | c. ≤15% NGHC species | 3.2 | 3.0 | 2.9 | 3.0 | 3.0 |
| Purity by Non- | a. MP purity ≥85% | 97.1 | 97.3 | 97.3 | 97.2 | 97.2 |
| reduced MCE | total peak area | | | | | |
| | b. ≤10% LMW species | 2.4 | 2.2 | 2.2 | 2.3 | 2.4 |
| | c. % HMW species | 0.49 | 0.46 | 0.48 | 0.45 | 0.43 |
| Purity by | a. Purity ≥90% total | 99.0 | 98.9 | 99.0 | 98.9 | 98.9 |
| SE-UPLC | peak area | | | | | |
| | b. ≤5% LMW species | 0.0 | 0.1 | 0.1 | 0.1 | 0.1 |
| | c. ≤7% HMW species | 1.0 | 1.0 | 0.9 | 1.0 | 1.0 |
| Charge Variant | a. ≤75% Region 1 | 38.7 | 38.9 | 37.2 | 38.9 | 38.1 |
| Analysis | b. ≥25% Region 2 | 52.8 | 51.4 | 52.9 | 52.1 | 52.5 |
| by iCIEF | c. ≤25% Region 3 | 8.5 | 9.7 | 9.9 | 9.0 | 9.4 |
| Particulate | ≥10 μm: ≤6000 | 56 | 63 | 42 | NR | 81 |
| Matter | particles/container | | | | | |
| (light | ≥25 μm: ≤600 | 4 | 0 | 4 | NR | 0 |
| obscuration) | particles/container | | | | | |
| Particulate | 2 μm ≤ x < 10 μm | 65 | 2534 | 1289 | NR | 1235 |
| Matter | (particle/mL) | | | | | |
| (MFI ™) | ≥10 μm (particle/mL) | 0 | 9 | 9 | NR | 17 |
| | ≥25 μm (particle/mL) | 0 | 0 | 0 | NR | 0 |

NR indicates tests not required at the time point, and NA indicates tests required at time points where data has not yet been collected.

Stability studies were also performed to evaluate the storage and accelerated stability of high concentration formulations (120 mg/mL or 174 mg/ml mAb0 in 10 mM L-histidine, pH 6.0, 10% (w/v) sucrose, 0.1% (w/v) polysorbate 80) stored in 6R Schott Type 1 borosilicate glass vials. 2.5 mL of the formulation was filled in 6R Type 1 borosilicate glass vials and incubated under storage, accelerated and stress conditions. The accelerated and stress conditions were selected to simulate the conditions beyond which the formulation is not expected to be subjected during manufacturing and handling. mAb1 was physically and chemically stable when stored at storage condition (2-8° C.) for at least 9 months. The formulation can withstand short exposures to refrigeration or room temperature and can be frozen and thawed without compromising either the physical or chemical stability of the protein. The formulation is stable to interfacial stress and can withstand agitation. In available data, no appreciable changes were detected in any of the monitored mAb1 formulation quality attributes after storage at 2-8° C. The results are shown in Tables 23-30.

TABLE 23
Stability of mAb1 Stored at 2-8° C.

| Formulation | 120.0 mg/mL mAb1 in a sterile aqueous buffered solution at pH 6.0 | | | | | |
|---|---|---|---|---|---|---|
| | containing 10 mM histidine, 10% (w/v) sucrose, 0.1% (w/v) polysorbate 80 | | | | | |
| Storage Container | USP/EP Type 1 borosilicate 6R ISO glass vial; 20 mm West S10-F451 4432/50 GRY B2-40 FluroTec ®- | | | | | |
| | coated chlorobutyl liquid stopper; West 20 mm aluminum seal with flip-off button | | | | | |
| Storage Condition | 5° C., upright | | | | | |
| | Quality | | | | | |
| | Acceptance | Length of Storage (months) | | | | |
| Assay | Criteria | t = 0 | 1 | 3 | 6 | 9 |
| Physical | Liquid | LEFVP | LEFVP | LEFVP | LEFVP | LEFVP |
| Form/ | essentially | | | | | |
| Condition | free from | | | | | |
| | visible | | | | | |
| | particulates | | | | | |
| Clarity | Not more | Not >IV | Not >III | Not >III | Not >IV | Not >IV |
| | turbid than | | | | | |
| | reference | | | | | |
| | suspension IV | | | | | |
| Color | Not more | Not >BY4 | Not >BY4 | Not >BY4 | Not >BY4 | Not >BY4 |
| | intensely | | | | | |
| | colored than | | | | | |
| | reference | | | | | |
| | solution BY2 | | | | | |
| pH | 5.7 to 6.3 | 6.0 | 6.1 | 6.1 | 6.0 | 6.1 |
| Total Protein | 108 to 132 | 120.3 | 121.3 | 123.0 | 121.5 | 122.2 |
| | mg/mL | | | | | |
| Potency by | 50% to 150% | 92 | NR | NR | 100 | NR |
| ELISA | of reference | | | | | |
| | standard | | | | | |
| Potency by | Report for | 70 | NR | NR | 85 | NR |
| Bioassay | information | | | | | |
| Purity by | a. % Purity ≥85% | 96.1 | 95.2 | 96.3 | 95.4 | 95.9 |
| Reduced | total peak area | | | | | |
| MCE | b. ≤10% | 0.7 | 0.9 | 0.6 | 0.8 | 0.8 |
| | LMW species | | | | | |
| | c. ≤15% | 2.7 | 3.3 | 2.6 | 3.2 | 2.9 |
| | NGHC species | | | | | |
| Purity by | a. MP purity ≥85% | 97.0 | 96.8 | 96.4 | 96.0 | 95.7 |
| Non- | total peak area | | | | | |
| reduced | b. ≤10% | 2.6 | 2.9 | 3.3 | 3.2 | 3.3 |
| MCE | LMW species | | | | | |
| | c. ≤5% HMW | 0.5 | 0.3 | 0.3 | 0.7 | 0.9 |
| | species | | | | | |
| Purity by | a. Purity ≥90% | 98.8 | 98.6 | 98.5 | 98.4 | 98.3 |
| SE-UPLC | total peak area | | | | | |
| | b. ≤5% LMW | 0.0 | 0.0 | 0.1 | 0.0 | 0.0 |
| | species | | | | | |
| | c. ≤7% HMW | 1.2 | 1.4 | 1.5 | 1.6 | 1.7 |
| | species | | | | | |
| Charge | a. ≤75% | 39.6 | 38.0 | 39.4 | 39.1 | 39.8 |
| Variant | Region 1 | | | | | |
| Analysis | b. ≥25% | 50.7 | 51.1 | 48.1 | 51.4 | 50.5 |
| by iCIEF | Region 2 | | | | | |
| | c. ≤25% | 9.6 | 10.9 | 12.4 | 9.5 | 9.7 |
| | Region 3 | | | | | |
| Charge | a. Report % | 34.3 | NR | NR | 33.6 | NR |
| Variant | Region 1 | | | | | |
| Analysis | b. Report % | 51.7 | NR | NR | 53.3 | NR |
| by CEX | Region 2 | | | | | |
| | c. Report % | 14.0 | NR | NR | 13.1 | NR |
| | Region 3 | | | | | |
| Particulate | ≥10 μm: ≤6000 | 3 | NR | 25 | 21 | NR |
| Matter by | (particles/ | | | | | |
| HIAC | container) | | | | | |
| (light | ≥25 μm: ≤600 | 0 | NR | 1 | 0 | NR |
| obscuration) | (particles/ | | | | | |
| | container) | | | | | |
| Particulate | 2 μm ≤ x < 10 μm | 144 | NR | 3491 | 8647 | NR |
| Matter by | (particle/mL) | | | | | |
| MFI ™ | ≥10 μm | 17 | NR | 30 | 57 | NR |
| Report | (particle/mL) | | | | | |
| particle/mL | ≥25 μm | 11 | NR | 3 | 3 | NR |
| | (particle/mL) | | | | | |
| Polysorbate | 0.1 ± 0.05% | 0.11 | NR | NR | 0.12 | NR |
| 80 Content | PS80 | | | | | |

| | Quality | | | | | |
|---|---|---|---|---|---|---|
| | Acceptance | Length of Storage (months) | | | | |
| Assay | Criteria | 12 | 18 | 24 | 36 | 48 |
| Physical | Liquid | LEFVP | LEFVP | LEFVP | LEFVP | LEFVP |
| Form/ | essentially | | | | | |
| Condition | free from | | | | | |
| | visible | | | | | |
| | particulates | | | | | |
| Clarity | Not more | Not >IV | Not >III | Not >III | Not >III | Not >III |
| | turbid than | | | | | |
| | reference | | | | | |
| | suspension IV | | | | | |
| Color | Not more | Not >BY4 | Not >BY4 | Not >BY4 | Not >BY4 | Not >BY4 |
| | intensely | | | | | |
| | colored than | | | | | |
| | reference | | | | | |
| | solution BY2 | | | | | |
| pH | 5.7 to 6.3 | 6.1 | 6.0 | 6.0 | 6.1 | 6.0 |
| Total Protein | 108 to 132 | 124.2 | 121.7 | 121.5 | 121.1 | 122.0 |
| | mg/mL | | | | | |
| Potency by | 50% to 150% | 83 | NR | 99 | 79 | 77 |
| ELISA | of reference | | | | | |
| | standard | | | | | |
| Potency by | Report for | 96 | NR | 99 | 90 | 98 |
| Bioassay | information | | | | | |
| Purity by | a. % Purity ≥85% | 95.4 | 95.9 | 95.3 | 94.9 | 93.9 |
| Reduced | total peak area | | | | | |
| MCE | b. ≤10% | 0.9 | 0.8 | 0.7 | 1.1 | 1.4 |
| | LMW species | | | | | |
| | c. ≤15% | 3.2 | 2.7 | 3.3 | 3.3 | 4.0 |
| | NGHC species | | | | | |
| Purity by | a. MP purity ≥85% | 96.6 | 96.4 | 95.9 | 95.8 | 95.2 |
| Non- | total peak area | | | | | |
| reduced | b. ≤10% | 2.6 | 2.4 | 3.0 | 2.9 | 3.4 |
| MCE | LMW species | | | | | |
| | c. ≤5% HMW | 0.9 | 1.2 | 1.1 | 1.3 | 1.4 |
| | species | | | | | |
| Purity by | a. Purity ≥90% | 98.1 | 98.2 | 97.9 | 97.7 | 97.6 |
| SE-UPLC | total peak area | | | | | |
| | b. ≤5% LMW | 0.1 | 0.0 | 0.1 | 0.1 | 0.1 |
| | species | | | | | |
| | c. ≤7% HMW | 1.8 | 1.8 | 2.0 | 2.2 | 2.3 |
| | species | | | | | |
| Charge | a. ≤75% | 40.9 | 39.4 | 39.2 | 40.5 | 41.7 |
| Variant | Region 1 | | | | | |
| Analysis | b. ≥25% | 49.0 | 49.7 | 50.9 | 50.0 | 47.6 |
| by iCIEF | Region 2 | | | | | |
| | c. ≤25% | 10.1 | 10.9 | 10.0 | 9.5 | 10.6 |
| | Region 3 | | | | | |
| Charge | a. Report % | 33.4 | NR | 33.8 | 34.6 | 37.6 |
| Variant | Region 1 | | | | | |
| Analysis | b. Report % | 53.8 | NR | 52.7 | 51.5 | 50.9 |
| by CEX | Region 2 | | | | | |
| | c. Report % | 12.8 | NR | 13.5 | 13.9 | 11.5 |
| | Region 3 | | | | | |
| Particulate | ≥10 μm: ≤6000 | 30 | NR | 29 | 20 | 44 |
| Matter by | (particles/ | | | | | |
| HIAC | container) | | | | | |
| (light | ≥25 μm: ≤600 | 0 | NR | 2 | 4 | 15 |
| obscuration) | (particles/ | | | | | |
| | container) | | | | | |
| Particulate | 2 μm ≤ x < 10 μm | 3109 | NR | 5736 | 1758 | 3132 |
| Matter by | (particle/mL) | | | | | |
| MFI ™ | ≥10 μm | 65 | NR | 71 | 21 | 26 |
| Report | (particle/mL) | | | | | |
| particle/mL | ≥25 μm | 3 | NR | 0 | 5 | 0 |
| | (particle/mL) | | | | | |
| Polysorbate | 0.1 ± 0.05% | 0.12 | NR | 0.11 | 0.11 | 0.11 |
| 80 Content | PS80 | | | | | |

TABLE 24
Stability of mAb1 formulation Incubated at 25° C./60% RH and 40° C./75% RH

| | |
|---|---|
| Formulation | 120.0 mg/mL mAb1 in a sterile aqueous buffered solution at pH 6.0 containing 10 |
| | mM histidine, 10% (w/v) sucrose, 0.1% (w/v) polysorbate 80 |
| Storage Container | USP/EP Type 1 borosilicate 6R ISO glass vial; 20 mm West S10-F451 4432/50 GRY B2-40 FluroTec ®- |
| | coated chlorobutyl liquid stopper; West 20 mm aluminum seal with flip-off button |

| | | Storage Condition | | | | | | | | |
|---|---|---|---|---|---|---|---|---|---|---|
| | Quality | No | 25° C./60% RH Storage | | | | 40° C./75% RH Storage | | | |
| | Acceptance | Storage | (months) | | | | (months) | | | |
| Assay | Criteria | t = 0 | 0.5 | 1 | 3 | 6 | 0.25 | 0.5 | 1 | 3 |
| Physical | Liquid | LEFVP | LEFVP | LEFVP | LEFVP | LEFVP | LEFVP | LEFVP | LEFVP | LEFVP |
| Form/ | essentially | | | | | | | | | |
| Condition | free from | | | | | | | | | |
| | visible | | | | | | | | | |
| | particulates | | | | | | | | | |
| Clarity | Not more | Not >IV | Not >IV | Not >III | Not >III | Not >IV | Not >IV | Not >IV | Not >III | Not >IV |
| | turbid than | | | | | | | | | |
| | reference | | | | | | | | | |
| | suspension | | | | | | | | | |
| | IV | | | | | | | | | |
| Color | Not more | Not >BY4 | Not >BY4 | Not >BY4 | Not >BY4 | Not >BY4 | Not >BY4 | Not >BY4 | Not >BY4 | Not >BY4 |
| | intensely | | | | | | | | | |
| | colored than | | | | | | | | | |
| | reference | | | | | | | | | |
| | solution BY2 | | | | | | | | | |
| pH | 5.7 to 6.3 | 6.0 | 6.0 | 6.1 | 6.1 | 6.0 | 6.0 | 6.0 | 6.1 | 6.1 |
| Total | 108 to 132 | 120.3 | 120.7 | 120.0 | 121.8 | 120.6 | 123.3 | 123.5 | 121.1 | 122.9 |
| Protein | mg/mL | | | | | | | | | |
| Potency | 50% to | 92 | NR | NR | NR | 85 | NR | NR | NR | 51 |
| by ELISA | 150% of | | | | | | | | | |
| | reference | | | | | | | | | |
| | standard | | | | | | | | | |
| Purity by | a. % Purity ≥85% | 96.1 | 95.6 | 96.5 | 94.9 | 94.9 | 95.1 | 93.9 | 93.4 | 91.9 |
| Reduced | total peak area | | | | | | | | | |
| MCE | b. ≤10% | 0.7 | 1.0 | 0.2 | 1.2 | 1.2 | 1.2 | 1.3 | 2.1 | 3.9 |
| | LMW species | | | | | | | | | |
| | c. ≤15% | 2.7 | 3.0 | 2.6 | 3.2 | 3.1 | 3.3 | 4.0 | 3.6 | 3.0 |
| | NGHC species | | | | | | | | | |
| Purity by | a. MP purity ≥85% | 97.0 | 96.2 | 96.6 | 95.1 | 94.4 | 96.6 | 95.3 | 94.1 | 89.9 |
| Non- | total peak area | | | | | | | | | |
| reduced | b. ≤10% | 2.6 | 2.8 | 3.0 | 4.1 | 4.1 | 2.9 | 3.2 | 4.0 | 7.5 |
| MCE | LMW species | | | | | | | | | |
| | c. % HMW | 0.5 | 1.0 | 0.4 | 0.8 | 1.6 | 0.4 | 1.3 | 1.9 | 2.8 |
| | species | | | | | | | | | |
| Purity by | a. Purity ≥90% | 98.8 | 98.3 | 98.0 | 97.7 | 97.1 | 97.9 | 97.4 | 96.5 | 92.9 |
| SE-UPLC | total peak area | | | | | | | | | |
| | b. ≤5% | 0.0 | 0.1 | 0.1 | 0.1 | 0.1 | 0.1 | 0.1 | 0.1 | 0.4 |
| | LMW species | | | | | | | | | |
| | c. ≤7% | 1.2 | 1.6 | 1.9 | 2.2 | 2.8 | 2.0 | 2.5 | 3.4 | 6.7 |
| | HMW species | | | | | | | | | |
| Charge | a. ≤75% | 39.6 | 44.8 | 38.9 | 43.1 | 45.9 | 50.1 | 52.9 | 58.7 | 77.2 |
| Variant | Region 1 | | | | | | | | | |
| Analysis | b. ≥25% | 50.7 | 45.3 | 50.2 | 44.9 | 43.3 | 39.3 | 36.6 | 30.8 | 14.8 |
| by iCIEF | Region 2 | | | | | | | | | |
| | c. ≤25% | 9.6 | 9.9 | 10.9 | 12.0 | 10.8 | 10.6 | 10.5 | 10.5 | 8.0 |
| | Region 3 | | | | | | | | | |
| Charge | a. % Region 1 | 34.3 | NR | NR | NR | 37.1 | NR | NR | NR | 59.7 |
| Variant | b. % Region 2 | 51.7 | NR | NR | NR | 49.7 | NR | NR | NR | 24.7 |
| Analysis | c. % Region 3 | 14.0 | NR | NR | NR | 13.2 | NR | NR | NR | 15.7 |
| by CEX | | | | | | | | | | |
| Particulate | ≥10 μm: ≤6000 | 3 | NR | NR | 18 | 5 | NR | NR | NR | 14 |
| Matter | particles/ | | | | | | | | | |
| (light | container | | | | | | | | | |
| obscuration) | ≥25 μm: ≤600 | 0 | NR | NR | 0 | 0 | NR | NR | NR | 0 |
| | particles/ | | | | | | | | | |
| | container | | | | | | | | | |
| Particulate | 2 μm ≤ x < 10 μm | 144 | NR | NR | 9775 | 6873 | NR | NR | NR | 16687 |
| Matter | (particle/mL) | | | | | | | | | |
| (MFI ™) | ≥10 μm | 17 | NR | NR | 946 | 63 | NR | NR | NR | 1835 |
| | (particle/mL) | | | | | | | | | |
| | ≥25 μm | 11 | NR | NR | 230 | 0 | NR | NR | NR | 361 |
| | (particle/mL) | | | | | | | | | |
| Polysorbate | Report % PS80 | 0.11 | NR | NR | NR | 0.11 | NR | NR | NR | 0.11 |
| 80 Content | | | | | | | | | | |

TABLE 25
Stability of mAb1 formulation after Agitation and Freeze-Thaw Stress

| | |
|---|---|
| Formulation | 120.0 mg/mL mAb1 in a sterile aqueous buffered solution at pH 6.0 containing 10 mM |
| | histidine, 10% (w/V) sucrose, 0.1% (w/v) polysorbate 80 |
| Storage Container | USP/EP Type 1 borosilicate 6R ISO glass vial; 20 mm West S10-F451 4432/50 GRY B2- |
| | 40 FluroTec ®-coated chlorobutyl liquid stopper; West 20 mm aluminum seal with flip- |
| | off button |

| | | Storage Condition | | | | |
|---|---|---|---|---|---|---|
| | | | 1000 RPM | 250 RPM | | |
| | Quality | No | Upright | Sideways | Freeze-Thaw | |
| | Acceptance | Storage | (minutes) | (hours) | Cycles | |
| Assay | Criteria | t = 0 | 120 | 48 | 2 | 4 |
| Physical Form/ | Liquid essentially | LEFVP | LEFVP | LEFVP | LEFVP | LEFVP |
| Condition | free from visible | | | | | |
| | particulates | | | | | |
| Clarity | Not more turbid | Not >IV | Not >IV | Not >III | Not >III | Not >III |
| | than reference | | | | | |
| | suspension IV | | | | | |
| Color | Not more intensely | Not >BY4 | Not >BY4 | Not >BY4 | Not >BY4 | Not >BY4 |
| | colored than | | | | | |
| | reference solution | | | | | |
| | BY2 | | | | | |
| pH | 5.7 to 6.3 | 6.0 | 6.1 | 6.1 | 6.1 | 6.1 |
| Total Protein | 108 to 132 mg/mL | 120.3 | 119.5 | 121.9 | 122.8 | 123.9 |
| Potency by ELISA | 50% to 150% of | 92 | 90 | 100 | NR | 85 |
| | reference standard | | | | | |
| Potency by | 50% to 150% of | 70 | 93 | 127 | NR | 97 |
| Bioassay | reference standard | | | | | |
| Purity by | a. % Purity ≥85% | 96.1 | 94.9 | 95. | 96.1 | 95.4 |
| Reduced MCE | total peak area | | | | | |
| | b. ≤10% LMW | 0.7 | 0.9 | 0.8 | 0.3 | 0.8 |
| | species | | | | | |
| | c. ≤15% NGHC | 2.7 | 3.6 | 3.6 | 3.1 | 3.3 |
| | species | | | | | |
| Purity by Non- | a. MP purity ≥85% | 97.0 | 96.5 | 96.6 | 96.6 | 96.8 |
| reduced MCE | total peak area | | | | | |
| | b. ≤10% LMW | 2.6 | 3.1 | 2.8 | 3.0 | 2.7 |
| | species | | | | | |
| | c. % HMW species | 0.5 | 0.5 | 0.6 | 0.4 | 0.4 |
| Purity by SE-UPLC | a. Purity ≥90% | 98.8 | 98.7 | 98.7 | 98.8 | 98.8 |
| | total peak area | | | | | |
| | b. ≤5% LMW | 0.0 | 0.0 | 0.0 | 0.0 | 0.0 |
| | species | | | | | |
| | c. ≤7% HMW | 1.2 | 1.3 | 1.4 | 1.2 | 1.2 |
| | species | | | | | |
| Charge Variant | a. ≤75% Region 1 | 39.6 | 39.0 | 39.8 | 39.3 | 39.5 |
| Analysis by | b. ≥25% Region 2 | 50.7 | 50.3 | 49.4 | 49.7 | 49.3 |
| iCIEF | c. ≤25% Region 3 | 9.6 | 10.7 | 10.9 | 10.9 | 11.2 |
| Particulate Matter | ≥10 μm: ≤6000 | 3 | 6 | 8 | NR | 12 |
| (light obscuration) | particles/ | | | | | |
| | container | | | | | |
| | ≥25 μm: ≤600 | 0 | 0 | 0 | NR | 1 |
| | particles/ | | | | | |
| | container | | | | | |
| Particulate | 2 μm ≤ x < 10 μm | 144 | 2152 | 109 | NR | 875 |
| Matter (MFI ™) | (particle/mL) | | | | | |
| | ≥10 μm | 17 | 38 | 5 | NR | 11 |
| | (particle/mL) | | | | | |
| | ≥25 μm | 11 | 3 | 5 | NR | 3 |
| | (particle/mL) | | | | | |

TABLE 26
Stability of mAb1 Drug Substance at −80° C.

| | |
|---|---|
| Formulation | 173.8 mg/mL mAb1, 10 mM L-histidine, 5% sucrose (w/v), pH 6.0 |
| Fill Volume | 2.5 mL |
| Storage Container | 5 mL polycarbonate vial with silicone lined polyethylene screw cap |
| Storage Condition | −80° C., Upright |

| | Assay Acceptance | Length of Storage (months) | | | | |
|---|---|---|---|---|---|---|
| Assay | Criteria^{a} | t = 0 | 1 | 3 | 6 | 9 |
| Physical | Liquid essentially free | LEFVP | LEFVP | LEFVP | LEFVP | LEFVP |
| Form/Condition | from visible particulates | | | | | |
| Clarity | Not more turbid than | Not >III | Not >III | Not >III | Not >III | Not >III |
| | reference suspension IV | | | | | |
| Color | Not more intensely | Not >BY4 | Not >BY4 | Not >BY4 | Not >BY4 | Not >BY4 |
| | colored than reference | | | | | |
| | solution BY2 | | | | | |
| pH | 5.8-6.2 | 6.0 | 6.1 | 6.1 | 6.1 | 6.1 |
| Total Protein | 156.4 to 191.2 mg/mL | 173.8 | 171.4 | 174.8 | 173.0 | 170.9 |
| Potency by | 50%-150% of reference | 68 | NR | NR | 62 | NR |
| Bioassay | standard | | | | | |
| Purity by | a. % Purity ≥85% | 95.8 | 95.8 | 95.6 | 95.7 | 96.3 |
| Reduced | total peak area | | | | | |
| MCE | b. ≤10% LMW species | 0.6 | 0.6 | 0.8 | 0.7 | 0.6 |
| | c. ≤15% NGHC species | 3.1 | 3.0 | 3.1 | 3.2 | 2.6 |
| Purity by Non- | a. MP purity ≥85% | 96.8 | 96.7 | 96.5 | 96.4 | 96.7 |
| reduced MCE | total peak area | | | | | |
| | b. ≤10% LMW species | 2.5 | 2.8 | 2.9 | 3.1 | 2.8 |
| | c. % HMW species | 0.7 | 0.6 | 0.6 | 0.6 | 0.5 |
| Purity by | a. Purity ≥90% | 98.4 | 98.6 | 98.6 | 98.7 | 98.7 |
| SE-UPLC | total peak area | | | | | |
| | b. ≤5% LMW species | 0.1 | 0.1 | 0.0 | 0.0 | 0.0 |
| | c. ≤7% HMW species | 1.5 | 1.3 | 1.3 | 1.3 | 1.3 |
| Charge Variant | a. ≤75% Region 1 | 40.1 | 40.6 | 39.8 | 38.3 | 38.7 |
| Analysis | b. ≥25% Region 2 | 50.8 | 49.6 | 49.5 | 51.9 | 51.5 |
| by iCIEF | c. ≤25% Region 3 | 9.2 | 9.8 | 10.7 | 9.7 | 9.8 |
| Charge Variant | a. % Region 1 | 33.9 | NR | NR | 33.4 | NR |
| Analysis | b. % Region 2 | 53.5 | NR | NR | 54.6 | NR |
| by CEX | c. % Region 3 | 12.5 | NR | NR | 12.1 | NR |
| Particulate | 2 μm ≤ x < 10 μm | 98 | NR | 650 | 82 | NR |
| Matter | (particle/mL) | | | | | |
| (MFI ™) | ≥10 μm (particle/mL) | 34 | NR | 124 | 13 | NR |
| | ≥25 μm (particle/mL) | 9 | NR | 17 | 0 | NR |
| Polysorbate | Report for | 0.013 | NR | NR | NR | NR |
| 80 Content | information | | | | | |
| | Assay Acceptance | Length of Storage (months) | | | | |
| Assay | Criteria^{a} | 12 | 18 | 24 | 36 | 48 |
| Physical | Liquid essentially free | LEFVP | LEFVP | LEFVP | LEFVP | LEFVP |
| Form/Condition | from visible particulates | | | | | |
| Clarity | Not more turbid than | Not >III | Not >III | Not >III | Not >III | Not >III |
| | reference suspension IV | | | | | |
| Color | Not more intensely | Not >BY4 | Not >BY4 | Not >BY4 | Not >BY4 | Not >BY4 |
| | colored than reference | | | | | |
| | solution BY2 | | | | | |
| pH | 5.8-6.2 | 6.0 | 6.0 | 6.1 | 6.0 | 6.1 |
| Total Protein | 156.4 to 191.2 mg/mL | 176.5 | 173.2 | 169.7 | 169.1 | 169.6 |
| Potency by | 50%-150% of reference | 72 | NR | 85 | 82 | 89 |
| Bioassay | standard | | | | | |
| Purity by | a. % Purity ≥85% | 95.5 | NA | 96.0 | 95.4 | 95.3 |
| Reduced | total peak area | | | | | |
| MCE | b. ≤10% LMW species | 0.6 | NA | 0.3 | 0.8 | 1.2 |
| | c. ≤15% NGHC species | 3.3 | NA | 3.4 | 3.1 | 3.1 |
| Purity by Non- | a. MP purity ≥85% | 96.9 | 96.7 | 96.9 | 96.6 | 95.5 |
| reduced MCE | total peak area | | | | | |
| | b. ≤10% LMW species | 2.5 | 2.4 | 2.5 | 2.7 | 3.9 |
| | c. % HMW species | 0.6 | 0.9 | 0.6 | 0.8 | 0.7 |
| Purity by | a. Purity ≥90% | 98.7 | 98.7 | 98.7 | 98.7 | 98.7 |
| SE-UPLC | total peak area | | | | | |
| | b. ≤5% LMW species | 0.0 | 0.0 | 0.0 | 0.0 | 0.0 |
| | c. ≤7% HMW species | 1.3 | 1.3 | 1.3 | 1.3 | 1.3 |
| Charge Variant | a. ≤75% Region 1 | 40.2 | 38.1 | 38.7 | 37.8 | 38.2 |
| Analysis | b. ≥25% Region 2 | 49.4 | 52.1 | 52.4 | 51.4 | 51.2 |
| by iCIEF | c. ≤25% Region 3 | 10.4 | 9.8 | 8.9 | 10.8 | 10.6 |
| Charge Variant | a. % Region 1 | 34.1 | NR | 33.4 | 34.1 | 31.6 |
| Analysis | b. % Region 2 | 53.8 | NR | 53.9 | 53.5 | 54.3 |
| by CEX | c. % Region 3 | 12.1 | NR | 12.6 | 12.4 | 14.1 |
| Particulate | 2 μm ≤ x < 10 μm | 228 | NR | 199 | 132 | 478 |
| Matter | (particle/mL) | | | | | |
| (MFI ™) | ≥10 μm (particle/mL) | 11 | NR | 7 | 13 | 105 |
| | ≥25 μm (particle/mL) | 3 | NR | 3 | 3 | 15 |
| Polysorbate | Report for | NR | NR | NR | NR | NR |
| 80 Content | information | | | | | |

NR indicates tests not required at the time point.

TABLE 27
Stability of mAb1 Drug Substance at −30° C.

| | |
|---|---|
| Formulation | 173.8 mg/mL mAb1, 10 mM L-histidine, 5% sucrose (w/v), pH 6.0 |
| Fill Volume | 2.5 mL |
| Storage Container | 5 mL polycarbonate vial with silicone lined polyethylene screw cap |
| Storage Condition | −30° C., Upright |

| | Assay Acceptance | Length of Storage (months) | | | | |
|---|---|---|---|---|---|---|
| Assay | Criteria | t = 0 | 1 | 3 | 6 | 9 |
| Physical | Liquid essentially free from | LEFVP | LEFVP | LEFVP | LEFVP | LEFVP |
| Form/Condition | visible particulates | | | | | |
| Clarity | Not more turbid than | Not >III | Not >III | Not >III | Not >III | Not >III |
| | reference suspension IV | | | | | |
| Color | Not more intensely colored | Not >BY4 | Not >BY4 | Not >BY4 | Not >BY4 | Not >BY4 |
| | than reference | | | | | |
| | solution BY2 | | | | | |
| pH | 5.8 to 6.2 | 6.0 | 6.0 | 6.1 | 6.1 | 6.1 |
| Total Protein | 156.4 to 191.2 mg/mL | 173.8 | 173.2 | 170.8 | 171.4 | 173.4 |
| Potency by | 50%-150% of | 68 | NR | NR | 68 | NR |
| Bioassay | reference standard | | | | | |
| Purity by | a. % Purity ≥85% | 95.8 | 95.9 | 95.5 | 94.9 | 95.7 |
| Reduced | total peak area | | | | | |
| MCE | b. ≤10% LMW species | 0.6 | 0.5 | 0.8 | 0.9 | 0.7 |
| | c. ≤15% NGHC species | 3.1 | 2.9 | 3.3 | 3.8 | 3.2 |
| Purity by Non- | a. MP purity ≥85% | 96.8 | 96.2 | 96.4 | 96.2 | 96.5 |
| reduced MCE | total peak area | | | | | |
| | b. ≤10% LMW species | 2.5 | 2.8 | 3.0 | 3.2 | 3.0 |
| | c. % HMW species | 0.7 | 1.0 | 0.6 | 0.6 | 0.5 |
| Purity by | a. Purity ≥90% | 98.4 | 98.7 | 98.7 | 98.6 | 98.6 |
| SE-UPLC | total peak area | | | | | |
| | b. ≤5% LMW species | 0.1 | 0.0 | 0.0 | 0.0 | 0.1 |
| | c. ≤7% HMW species | 1.5 | 1.3 | 1.3 | 1.3 | 1.3 |
| Charge Variant | a. ≤75% Region 1 | 40.1 | 40.3 | 39.5 | 39.0 | 39.8 |
| Analysis | b. ≥5% Region 2 | 50.8 | 49.5 | 50.4 | 50.8 | 49.9 |
| by iCIEF | c. ≤25% Region 3 | 9.2 | 10.3 | 10.1 | 10.2 | 10.3 |
| Charge Variant | a. % Region 1 | 33.9 | NR | NR | 33.0 | NR |
| Analysis by CEX | b. % Region 2 | 53.5 | NR | NR | 55.1 | NR |
| | c. % Region 3 | 12.5 | NR | NR | 11.9 | NR |
| Particulate | 2 μm ≤ x < 10 μm | 98 | NR | 1076 | 86 | NR |
| Matter | (particle/mL) | | | | | |
| (MFI ™) | ≥10 μm (particle/mL) | 34 | NR | 42 | 26 | NR |
| | ≥25 μm (particle/mL) | 9 | NR | 0 | 11 | NR |
| Polysorbate | Report for | 0.013 | NR | NR | NR | NR |
| 80 Content | information | | | | | |
| | Assay Acceptance | Length of Storage (months) | | | | |
| Assay | Criteria | 12 | 18 | 24 | 36 | 48 |
| Physical | Liquid essentially free from | LEFVP | LEFVP | LEFVP | LEFVP | LEFVP |
| Form/Condition | visible particulates | | | | | |
| Clarity | Not more turbid than | Not >III | Not >III | Not >III | Not >III | Not >III |
| | reference suspension IV | | | | | |
| Color | Not more intensely colored | Not >BY4 | Not >BY4 | Not >BY4 | Not >BY4 | Not >BY4 |
| | than reference | | | | | |
| | solution BY2 | | | | | |
| pH | 5.8 to 6.2 | 6.0 | 6.1 | 6.0 | 6.0 | 6.1 |
| Total Protein | 156.4 to 191.2 mg/mL | 176.9 | 169.8 | 170.4 | 170.1 | 169.0 |
| Potency by | 50%-150% of | 90 | NR | 85 | 94 | 102 |
| Bioassay | reference standard | | | | | |
| Purity by | a. % Purity ≥85% | 95.1 | NA | 95.4 | 96.2 | 95.7 |
| Reduced | total peak area | | | | | |
| MCE | b. ≤10% LMW species | 0.5 | NA | 0.7 | 0.6 | 0.8 |
| | c. ≤15% NGHC species | 3.8 | NA | 3.4 | 2.7 | 3.1 |
| Purity by Non- | a. MP purity ≥85% | 97.1 | 96.7 | 96.2 | 96.6 | 96.0 |
| reduced MCE | total peak area | | | | | |
| | b. ≤10% LMW species | 2.3 | 2.2 | 2.8 | 2.7 | 3.2 |
| | c. % HMW species | 0.6 | 1.1 | 1.1 | 0.7 | 0.7 |
| Purity by | a. Purity ≥90% | 98.6 | 98.5 | 98.6 | 98.6 | 98.6 |
| SE-UPLC | total peak area | | | | | |
| | b. ≤5% LMW species | 0.0 | 0.1 | 0.0 | 0.0 | 0.0 |
| | c. ≤7% HMW species | 1.4 | 1.4 | 1.4 | 1.4 | 1.4 |
| Charge Variant | a. ≤75% Region 1 | 40.3 | 39.0 | 38.8 | 38.1 | 37.3 |
| Analysis | b. ≥5% Region 2 | 49.2 | 50.9 | 51.8 | 51.8 | 52.5 |
| by iCIEF | c. ≤25% Region 3 | 10.5 | 10.1 | 9.4 | 10.1 | 10.2 |
| Charge Variant | a. % Region 1 | 33.5 | NR | 33.5 | 33.9 | 31.7 |
| Analysis by CEX | b. % Region 2 | 54.6 | NR | 53.7 | 53.6 | 54.2 |
| | c. % Region 3 | 11.9 | NR | 12.8 | 12.5 | 14.0 |
| Particulate | 2 μm ≤ x < 10 μm | 211 | NR | 140 | 153 | 290 |
| Matter | (particle/mL) | | | | | |
| (MFI ™) | ≥10 μm (particle/mL) | 26 | NR | 32 | 19 | 32 |
| | ≥25 μm (particle/mL) | 0 | NR | 7 | 9 | 3 |
| Polysorbate | Report for | NR | NR | NR | NR | NR |
| 80 Content | information | | | | | |

NR indicates tests not required at the time point.

TABLE 28
Stability of mAb1 Drug Substance at −20° C.

| | |
|---|---|
| Formulation | 173.8 mg/mL mAb1, 10 mM L-histidine, 5% sucrose (w/v), pH 6.0 |
| Fill Volume | 2.5 mL |
| Storage Container | 5 mL polycarbonate vial with silicone lined polyethylene screw cap |
| Storage Condition | −20° C., Upright |

| | Assay Acceptance | Length of Storage (months) | | | | | |
|---|---|---|---|---|---|---|---|
| Assay | Criteria^{a} | t = 0 | 1 | 3 | 6 | 9 | 12 |
| Physical | Liquid essentially free from | LEFVP | LEFVP | LEFVP | LEVFP | LEVFP | LEFVP |
| Form/Condition | visible particulates | | | | | | |
| Clarity | Not more turbid than | Not >III | Not >III | Not >III | Not >III | Not >III | Not >III |
| | reference suspension IV | | | | | | |
| Color | Not more intensely colored | Not >BY4 | Not >BY4 | Not >BY4 | Not >BY4 | Not >BY4 | Not >BY4 |
| | than reference solution | | | | | | |
| | BY2 | | | | | | |
| pH | 5.8 to 6.2 | 6.0 | 6.0 | 6.1 | 6.1 | 6.1 | 6.0 |
| Total Protein | 156.4 to 191.2 mg/mL | 173.8 | 171.1 | 173.5 | 170.4 | 173.9 | 174.0 |
| Potency by | 50%-150% of reference | 68 | NR | NR | 62 | NR | 82 |
| Bioassay | standard | | | | | | |
| Purity by | a. % Purity ≥85% | 95.8 | 96.8 | 96.3 | 95.6 | 95.3 | 95.4 |
| Reduced | total peak area | | | | | | |
| MCE | b. ≤10% LMW species | 0.6 | 0.4 | 0.6 | 0.5 | 0.7 | 0.7 |
| | c. ≤15% NGHC species | 3.1 | 2.3 | 2.6 | 3.3 | 3.5 | 3.3 |
| Purity by Non- | a. MP purity ≥85% | 96.8 | 96.6 | 96.5 | 96.2 | 96.6 | 97.0 |
| reduced MCE | total peak area | | | | | | |
| | b. ≤ 10% LMW species | 2.5 | 2.7 | 3.0 | 3.1 | 2.9 | 2.3 |
| | c. % HMW species | 0.7 | 0.7 | 0.5 | 0.7 | 0.5 | 0.7 |
| Purity by | a. Purity ≥90% | 98.4 | 98.7 | 98.6 | 98.6 | 98.6 | 98.5 |
| SE-UPLC | total peak area | | | | | | |
| | b. ≤5% LMW species | 0.1 | 0.0 | 0.0 | 0.0 | 0.1 | 0.1 |
| | c. ≤7% HMW species | 1.5 | 1.3 | 1.4 | 1.4 | 1.4 | 1.4 |
| Charge Variant | a. ≤75% Region 1 | 40.1 | 41.4 | 39.9 | 38.9 | 39.4 | 39.5 |
| Analysis | b. ≥25% Region 2 | 50.8 | 48.5 | 49.0 | 50.3 | 50.6 | 50.2 |
| by iCIER | c. ≤25% Region 3 | 9.2 | 10.1 | 11.1 | 10.8 | 10.0 | 10.2 |
| Charge Variant | a. % Region 1 | 33.9 | NR | NR | 33.0 | NR | 33.6 |
| Analysis by CEX | b. % Region 2 | 53.5 | NR | NR | 54.8 | NR | 54.4 |
| | c. % Region 3 | 12.5 | NR | NR | 12.2 | NR | 12.0 |
| Particulate | 2 μm ≤ x < 10 μm | 98 | NR | 554 | 78 | NR | 149 |
| Matter | (particle/mL) | | | | | | |
| (MFI ™) | ≥10 μm (particle/mL) | 34 | NR | 101 | 11 | NR | 11 |
| | ≥25 μm (particle/mL) | 9 | NR | 21 | 0 | NR | 11 |
| Polysorbate | Report for | 0.013 | NR | NR | NR | NR | NR |
| 80 Content | information | | | | | | |

NR indicates tests not required at the time point.

TABLE 29
Stability of mAb1 Drug Substance Incubated at 5° C., 25° C./60% RH and 40° C./75% RH

| | |
|---|---|
| Formulation | 173.8 mg/mL mAb1, 10 mM L-histidine, 5% sucrose (w/v), pH 6.0 |
| Fill Volume | 2.5 mL |
| Storage Container | 5 mL polycarbonate vial with silicone lined polyethylene screw cap |

| | | Storage Condition | | | | | |
|---|---|---|---|---|---|---|---|
| | Assay | | 5° C. Storage | | | 25° C./60% RH Storage | |
| | Acceptance | NA | (months) | | | (months) | |
| Assay | Criteria | t = 0 | 1 | 3 | 6 | 1 | 3 |
| Physical | Liquid essentially | LEFVP | LEFVP | LEFVP | LEFVP | LEFVP | LEFVP |
| Form/ | free from visible | | | | | | |
| Condition | particulates | | | | | | |
| Clarity | Not more turbid | Not >III | Not >III | Not >III | Not >III | Not >III | Not >III |
| | than reference | | | | | | |
| | suspension IV | | | | | | |
| Color | Not more | Not >BY4 | Not >BY4 | Not >BY4 | Not >BY4 | Not >BY4 | Not >BY4 |
| | intensely colored | | | | | | |
| | than reference | | | | | | |
| | solution BY2 | | | | | | |
| pH | 5.8 to 6.2 | 6.0 | 6.1 | 6.1 | 6.1 | 6.1 | 6.1 |
| Total | 156.4 to 191.2 | 173.8 | 174.8 | 173.7 | 176.4 | 174.0 | 179.3 |
| Protein | mg/mL | | | | | | |
| Purity by | a. % Purity ≥85% | 95.8 | 96.7 | 96.0 | 95.3 | 96.0 | 95.4 |
| Reduced | total peak area | | | | | | |
| MCE | b. ≤10% LMW | 0.6 | 0.6 | 0.7 | 0.8 | 0.8 | 1.0 |
| | species | | | | | | |
| | c. ≤15% NGHC | 3.1 | 2.2 | 2.7 | 3.3 | 2.4 | 2.9 |
| | species | | | | | | |
| Purity by | a. MP purity ≥85% | 96.8 | 96.5 | 96.2 | 95.8 | 96.1 | 95.1 |
| Non- | total peak area | | | | | | |
| reduced | b. ≤10% LMW | 2.5 | 2.8 | 3.0 | 3.2 | 2.8 | 3.4 |
| MCE | species | | | | | | |
| | c. % HMW | 0.7 | 0.7 | 0.8 | 1.0 | 1.1 | 1.5 |
| | species | | | | | | |
| Purity by | a. Purity ≥90% | 98.4 | 98.4 | 98.4 | 98.0 | 97.8 | 97.0 |
| SE-UPLC | total peak area | | | | | | |
| | b. ≤5% LMW | 0.1 | 0.1 | 0.0 | 0.0 | 0.0 | 0.1 |
| | species | | | | | | |
| | c. ≤7% HMW | 1.5 | 1.5 | 1.6 | 1.9 | 2.2 | 2.8 |
| | species | | | | | | |
| Charge | a. ≤75% | 40.1 | 41.7 | 40.4 | 38.3 | 41.3 | 43.9 |
| Variant | Region 1 | | | | | | |
| Analysis | b. ≥25% | 50.8 | 48.0 | 49.6 | 50.9 | 48.1 | 44.8 |
| by iCIEF | Region 2 | | | | | | |
| | c. ≤25% | 9.2 | 10.3 | 10.0 | 10.8 | 10.5 | 11.3 |
| | Region 3 | | | | | | |
| Charge | a. % Region 1 | 33.9 | NR | NF | 33.1 | NR | NR |
| Variant | b. % Region 2 | 53.5 | NR | NR | 54.7 | NR | NR |
| Analysis | c. % Region 3 | 12.5 | NR | NR | 12.2 | NR | NR |
| by CEX | | | | | | | |
| Particulate | 2 μm ≤ x < 10 μm | 98 | NR | 1214 | 138 | NR | 2787 |
| Matter | (particle/mL) | | | | | | |
| (MFI ™) | ≥10 μm | 34 | NR | 234 | 11 | NR | 320 |
| | (particle/mL) | | | | | | |
| | ≥25 μm | 9 | NR | 15 | 5 | NR | 26 |
| | (particle/mL) | | | | | | |
| Polysorbate | Report for | 0.013 | NR | NR | NR | NR | NR |
| 80 Content | information | | | | | | |

| | | Storage Condition | | | | |
|---|---|---|---|---|---|---|
| | Assay | 25° C./60% RH Storage | 40° C./75% RH Storage | | | |
| | Acceptance | (months) | (months) | | | |
| Assay | Criteria | 6 | 0.25 | 0.5 | 1 | 3 |
| Physical | Liquid essentially | LEFVP | LEFVP | LEFVP | LEFVP | LEFVP |
| Form/ | free from visible | | | | | |
| Condition | particulates | | | | | |
| Clarity | Not more turbid | Not >III | Not >III | Not >III | Not >III | Not >III |
| | than reference | | | | | |
| | suspension IV | | | | | |
| Color | Not more | Not >BY4 | Not >BY4 | Not >BY4 | Not >BY4 | Not >BY3 |
| | intensely colored | | | | | |
| | than reference | | | | | |
| | solution BY2 | | | | | |
| pH | 5.8 to 6.2 | 6.1 | 6.1 | 6.1 | 6.1 | 6.0 |
| Total | 156.4 to 191.2 | 185.9 | 168.6 | 175.4 | 177.0 | 183.9 |
| Protein | mg/mL | | | | | |
| Purity by | a. % Purity ≥85% | 93.8 | 94.7 | 94.2 | 94.7 | 91.6 |
| Reduced | total peak area | | | | | |
| MCE | b. ≤10% LMW | 1.6 | 1.2 | 1.3 | 0.7 | 3.4 |
| | species | | | | | |
| | c. ≤15% NGHC | 3.5 | 3.2 | 3.6 | 3.6 | 3.2 |
| | species | | | | | |
| Purity by | a. MP purity ≥85% | 93.9 | 95.7 | 94.8 | 94.0 | 89.8 |
| Non- | total peak area | | | | | |
| reduced | b. ≤10% LMW | 4.0 | 3.0 | 3.4 | 3.8 | 6.2 |
| MCE | species | | | | | |
| | c. % HMW | 2.2 | 1.4 | 1.8 | 2.2 | 4.1 |
| | species | | | | | |
| Purity by | a. Purity ≥90% | 96.3 | 97.2 | 96.5 | 95.5 | 90.3 |
| SE-UPLC | total peak area | | | | | |
| | b. ≤5% LMW | 0.1 | 0.1 | 0.2 | 0.1 | 0.5 |
| | species | | | | | |
| | c. ≤7% HMW | 3.6 | 2.7 | 3.4 | 4.3 | 9.2 |
| | species | | | | | |
| Charge | a. ≤75% | 46.1 | 43.3 | 44.8 | 60.7 | 77.4 |
| Variant | Region 1 | | | | | |
| Analysis | b. ≥25% | 43.6 | 46.4 | 42.2 | 29.1 | 15.8 |
| by iCIEF | Region 2 | | | | | |
| | c. ≤25% | 10.3 | 10.3 | 13.0 | 10.2 | 6.9 |
| | Region 3 | | | | | |
| Charge | a. % Region 1 | 37.4 | NR | NR | NR | 59.8 |
| Variant | b. % Region 2 | 49.1 | NR | NR | NR | 20.8 |
| Analysis | c. % Region 3 | 13.5 | NR | NR | NR | 19.3 |
| by CEX | | | | | | |
| Particulate | 2 μm ≤ x < 10 μm | 119 | NR | NR | NR | 978 |
| Matter | (particle/mL) | | | | | |
| (MFI ™) | ≥10 μm | 9 | NR | NR | NR | 217 |
| | (particle/mL) | | | | | |
| | ≥25 μm | 7 | NR | NR | NR | 23 |
| | (particle/mL) | | | | | |
| Polysorbate | Report for | NR | NR | NR | NR | NR |
| 80 Content | information | | | | | |

NR indicates tests not required at the time point.

TABLE 30
Stability of mAb1 Drug Substance - Effect of Agitation and Freese/Thaw

| | |
|---|---|
| Formulation | 173.8 mg/mL mAb1, 10 mM L-histidine, 5% sucrose (w/v), pH 6.0 |
| Fill Volume | 2.5 mL |
| Storage Container | 5 mL polycarbonate vial with silicone lined polyethylene screw cap |

| | | Storage Condition | | | | |
|---|---|---|---|---|---|---|
| | Assay Acceptance | No Stress | Agitation (minutes) | | Freeze/Thaw (cycles) | |
| Assay | Criteria | t = 0 | 60 | 120 | 2 | 4 |
| Physical | Liquid essentially | LEFVP | LEFVP | LEFVP | LEVFP | LEVFP |
| Form/ | free from visible | | | | | |
| Condition | particulates | | | | | |
| Clarity | Not more turbid than | Not >III | Not >III | Not >III | Not >III | Not >III |
| | reference | | | | | |
| | suspension IV | | | | | |
| Color | Not more intensely | Not >BY4 | Not >BY4 | Not >BY4 | Not >BY4 | Not >BY4 |
| | colored than | | | | | |
| | reference solution | | | | | |
| | BY2 | | | | | |
| pH | 5.8 to 6.2 | 6.0 | 6.1 | 6.1 | 6.1 | 6.1 |
| Total | 156.4 to 191.2 | 173.8 | 171.6 | 172.0 | 168.6 | 169.5 |
| Protein | mg/mL | | | | | |
| Purity by | a. % Purity ≥85% | 95.8 | 95.7 | 95.9 | 95.9 | 95.5 |
| Reduced | total peak area | | | | | |
| MCE | b. ≤10% LMW | 0.6 | 0.7 | 0.7 | 0.7 | 0.7 |
| | species | | | | | |
| | c. ≤15% NGHC | 3.1 | 3.0 | 2.8 | 3.0 | 3.2 |
| | species | | | | | |
| Purity by | a. MP purity ≥85% | 96.8 | 96.6 | 96.7 | 96.7 | 96.6 |
| Non- | total peak area | | | | | |
| reduced | b. ≤10% LMW | 2.5 | 2.7 | 2.8 | 2.8 | 2.7 |
| MCE | species | | | | | |
| | c. % HMW species | 0.7 | 0.6 | 0.6 | 0.6 | 0.7 |
| Purity by | a. Purity ≥90% | 98.4 | 98.8 | 98.7 | 98.6 | 98.4 |
| SE-UPLC | total peak area | | | | | |
| | b. ≤5% LMW | 0.1 | 0.0 | 0.0 | 0.0 | 0.1 |
| | species | | | | | |
| | c. ≤7% HMW | 1.5 | 1.2 | 1.3 | 1.4 | 1.5 |
| | species | | | | | |
| Charge | a. ≤75% Region 1 | 40.1 | 40.8 | 40.7 | 39.5 | 40.1 |
| Variant | b. ≥25% Region 2 | 50.8 | 49.6 | 50.3 | 51.2 | 50.2 |
| Analysis | c. ≤25% Region 3 | 9.2 | 9.6 | 9.0 | 9.3 | 9.7 |
| by iCIEF | | | | | | |
| Charge | a. % Region 1 | 33.9 | NR | 34.0 | NR | 34.2 |
| Variant | b. % Region 2 | 53.5 | NR | 53.4 | NR | 53.0 |
| Analysis | c. % Region 3 | 12.5 | NR | 12.6 | NR | 12.8 |
| by CEX | | | | | | |
| Particulate | Report particle/mL: | 98 | NR | 1928 | NR | 152 |
| Matter | 2 μm ≤ x < 10 μm | | | | | |
| (MFI ™) | ≥10 μm | 34 | NR | 69 | NR | 9 |
| | ≥25 μm | 9 | NR | 9 | NR | 5 |

NR indicates tests not required at the time point. NA indicates tests required at time points where data has not yet been collected.

### Example 5: Proven Acceptable Range Studies

This Example discusses the proven acceptable range (PAR) studies which were undertaken to test the robustness of the different mAb1 formulations.

To evaluate the impact of the individual formulation parameters on the long-term storage stability of mAb1 formulations, a D-optimal multivariate design with the addition of a center point (a formulation with all factors at the nominal level) was applied in the DOE study. The tested formulation parameter ranges were defined to be wider than or equal to the manufacturing acceptance criteria.

All tested formulations were characterized and assessed for physical and chemical properties and stability. The formulations were assessed for storage stability, and for stability under accelerated stress conditions. Evaluation of stability under accelerated and stress conditions was performed by subjecting the formulations to a variety of tests representative of extreme handling conditions that the formulations are not expected to exceed during manufacture and handling. Stability under additional stresses including agitation and freeze/thaw was also evaluated.

The analytical methods used to assess stability in the formulation development studies include color, appearance, clarity, pH, protein concentration by RP-UPLC, molecular purity by SE-UPLC or MCE, charge variant analysis by iCIEF or CEX, particle analysis by light obscuration by HIAC or microflow imaging, polysorbate 80 analysis by CAD.

For 5 mg Drug Product (2 mg/ml Concentration)

Table 31 lists the formulation parameters and ranges for the 5 mg drug product (2 mg/ml concentration). Table 32 lists the formulations tested.

All formulations were prepared and filled into 6R borosilicate glass vials with a 3.0 mL fill volume and assessed for long-term storage stability at 2-8° C., accelerated condition at 25° C. 60% RH, stress condition at 40° C. 75% RH and agitation at 250 RPM orbital shaking. The formulation interactions were analyzed by JMP (JMP® 13.2.1) using a D-optimal design.

TABLE 31
Summary of Formulation Parameters and
Formulation Ranges (for 2 mg/ml)

| Formulation | | | | | Range |
|---|---|---|---|---|---|
| Parameter | Target | | Range | | (%) |
| mAb1 | 2.0 | mg/mL | 1.8-2.2 | mg/mL | ±10% |
| L-histidine | 10 | mM | 8-12 | mM | ±20% |
| Sucrose | 10.0% | (w/v) | 8-12% | (w/v) | ±20% |
| Polysorbate 80 | 0.1% | (w/v) | 0.05-0.15% | (w/v) | ±50% |

| | | | |
|---|---|---|---|
| pH | 6.0 | 5.7-6.3 | ±0.3 |

TABLE 32
Formulations Tested in the Robustness
Study Based on D-optimal Design

| | | | | % | |
|---|---|---|---|---|---|
| | REGN5458 | L-Histidine | Sucrose | Polysorbate | |
| Formulation | (mg/mL) | (mM) | (% w/v) | 80 (w/v) | pH |
| F1 | 1.8 | 8 | 12 | 0.15 | 5.7 |
| F2 | 1.8 | 8 | 8 | 0.05 | 6.3 |
| F3 | 2.2 | 8 | 12 | 0.05 | 6.3 |
| F4 | 2.0 | 8 | 8 | 0.15 | 5.7 |
| F5 | 1.8 | 12 | 8 | 0.15 | 6.3 |
| F6 | 2.0 | 8 | 10 | 0.05 | 6.3 |
| F7 | 2.2 | 8 | 10 | 0.15 | 5.7 |
| F8 | 2.2 | 12 | 10 | 0.15 | 6.3 |
| F9 | 2.0 | 12 | 12 | 0.15 | 6.3 |
| F10 | 2.0 | 12 | 12 | 0.05 | 5.7 |
| F11 | 1.8 | 12 | 10 | 0.05 | 5.7 |
| F12 | 2.2 | 12 | 8 | 0.05 | 5.7 |
| F13 (target) | 2.0 | 10 | 10 | 0.1 | 6.0 |

For 50 mg Drug Product (10 mg/ml Concentration)

Table 33 lists the formulation parameters and ranges for the 50 mg drug product (10 mg/ml concentration). Table 34 lists the formulations tested.

For the 50 mg drug product (10 mg/ml concentration), all formulations were prepared and filled into 20 mL Type 1 glass vials with a 5.5 mL fill volume and assessed for long-term storage stability at 2-8° C., accelerated condition at 25° C. 60% RH, stress condition at 40° C. 75% RH and agitation at 250 RPM orbital shaking. The formulation interactions were analyzed by JMP (JMP® 13.2.1) using a D-optimal design.

TABLE 33
Summary of Formulation Parameters and
Formulation Ranges (for 10 mg/ml)

| Formulation | | | | | Range |
|---|---|---|---|---|---|
| Parameter | Target | | Range | | (%) |
| REGN5458 | 10.0 | mg/mL | 9.0-11.0 | mg/mL | ±10% |
| L-histidine | 10 | mM | 8-12 | mM | ±20% |
| Sucrose | 10.0% | (w/v) | 8-12% | (w/v) | ±20% |
| Polysorbate 80 | 0.1% | (w/v) | 0.05-0.15% | (w/v) | ±50% |

| | | | |
|---|---|---|---|
| pH | 6.0 | 5.7-6.3 | ±0.3 |

TABLE 34
Formulations Tested in the Robustness
Study Based on D-optimal Design

| | | | | % | |
|---|---|---|---|---|---|
| | REGN5458 | L-Histidine | Sucrose | Polysorbate | |
| Formulation | (mg/mL) | (mM) | (% w/v) | 80 (w/v) | pH |
| F1 | 9 | 8 | 12 | 0.15 | 5.7 |
| F2 | 9 | 8 | 8 | 0.05 | 6.3 |
| F3 | 11 | 8 | 12 | 0.05 | 6.3 |
| F4 | 10 | 8 | 8 | 0.15 | 5.7 |
| F5 | 9 | 12 | 8 | 0.15 | 6.3 |
| F6 | 10 | 8 | 10 | 0.05 | 6.3 |
| F7 | 11 | 8 | 10 | 0.15 | 5.7 |
| F8 | 11 | 12 | 10 | 0.15 | 6.3 |
| F9 | 10 | 12 | 12 | 0.15 | 6.3 |
| F10 | 10 | 12 | 12 | 0.05 | 5.7 |
| F11 | 9 | 12 | 10 | 0.05 | 5.7 |
| F12 | 11 | 12 | 8 | 0.05 | 5.7 |
| F13 (target) | 10 | 10 | 10 | 0.1 | 6.0 |

For 200 mg Drug Product (20 mg/ml Concentration)

Table 35 lists the formulation parameters and ranges for the 200 mg drug product (20 mg/ml concentration). Table 36 lists the formulations tested.

All formulations were prepared and filled into 20R Type 1 glass vials with a 10.5 mL fill volume and assessed for long-term storage stability at 2-8° C., accelerated condition at 25° C. 60% RH, stress condition at 40° C. 75% RH and agitation at 250 RPM orbital shaking. The formulation interactions were analyzed by JMP (JMP® 13.2.1) using a D-optimal design.

TABLE 35
Summary of Formulation Parameters and
Formulation Ranges (for 20 mg/ml)

| Formulation | | | | | Range |
|---|---|---|---|---|---|
| Parameter | Target | | Range | | (%) |
| REGN5458 | 20.0 | mg/mL | 18.0-22.0 | mg/mL | ±10% |
| L-histidine | 10 | mM | 8-12 | mM | ±20% |
| Sucrose | 10.0% | (w/v) | 8-12% | (w/v) | ±20% |
| Polysorbate 80 | 0.1% | (w/v) | 0.05-0.15% | (w/v) | ±50% |

| | | | |
|---|---|---|---|
| pH | 6.0 | 5.7-6.3 | ±0.3 |

TABLE 36
Formulations Tested in the Robustness
Study Based on D-optimal Design

| | | | | % | |
|---|---|---|---|---|---|
| | REGN5458 | L-Histidine | Sucrose | Polysorbate | |
| Formulation | (mg/mL) | (mM) | (% w/v) | 80 (w/v) | pH |
| F1 | 18 | 8 | 12 | 0.15 | 5.7 |
| F2 | 18 | 8 | 8 | 0.05 | 6.3 |
| F3 | 22 | 8 | 12 | 0.05 | 6.3 |
| F4 | 20 | 8 | 8 | 0.15 | 5.7 |
| F5 | 18 | 12 | 8 | 0.15 | 6.3 |
| F6 | 20 | 8 | 10 | 0.05 | 6.3 |
| F7 | 22 | 8 | 10 | 0.15 | 5.7 |
| F8 | 22 | 12 | 10 | 0.15 | 6.3 |
| F9 | 20 | 12 | 12 | 0.15 | 6.3 |
| F10 | 20 | 12 | 12 | 0.05 | 5.7 |
| F11 | 18 | 12 | 10 | 0.05 | 5.7 |
| F12 | 22 | 12 | 8 | 0.05 | 5.7 |
| F13 (target) | 20 | 10 | 10 | 0.1 | 6.0 |

For 120 mg/ml Concentration

Table 37 lists the formulation parameters and ranges for the 120 mg/m concentration formulation. Table 38 lists the formulations tested.

All formulations were prepared and filled into 5 mL polycarbonate vials with a 2.5 mL fill volume and assessed for long-term storage stability at −80° C. and 20° C., accelerated condition at 5° C. and 25° C. 60% RH, and freezing and thawing for 4 cycles. The formulation interactions were analyzed by JMP (JMP® 13.2.1) using a D-optimal design.

TABLE 37
Summary of Formulation Parameters and
Formulation Ranges in the DOE Study

| Formulation | | | | | Range |
|---|---|---|---|---|---|
| Parameter | Target | | Range | | (%) |
| REGN5458 | 120.0 | mg/mL | 108.0-132.0 | mg/mL | ±10% |
| L-histidine | 10 | mM | 8-12 | mM | ±20% |
| Sucrose | 10.0% | (w/v) | 8-12% | (w/v) | ±20% |
| Polysorbate 80 | 0.1% | (w/v) | 0.05-0.15% | (w/v) | ±50% |

| | | | |
|---|---|---|---|
| pH | 6.0 | 5.7-6.3 | ±0.3 |

TABLE 38
Formulations Tested in the Robustness
Study Based on D-optimal Design

| | | | | % | |
|---|---|---|---|---|---|
| | REGN5458 | L-Histidine | Sucrose | Polysorbate | |
| Formulation | (mg/mL) | (mM) | (% w/v) | 80 (w/v) | pH |
| F1 | 132 | 12 | 8 | 0.05 | 5.7 |
| F2 | 132 | 8 | 8 | 0.05 | 6.3 |
| F3 | 108 | 8 | 10 | 0.15 | 5.7 |
| F4 | 132 | 12 | 10 | 0.15 | 5.7 |
| F5 | 108 | 12 | 8 | 0.15 | 6.3 |
| F6 | 132 | 8 | 12 | 0.15 | 6.3 |
| F7 | 132 | 12 | 10 | 0.05 | 6.3 |
| F8 | 108 | 8 | 12 | 0.05 | 5.7 |
| F9 | 108 | 8 | 10 | 0.05 | 6.3 |
| F10 | 108 | 12 | 12 | 0.05 | 6.3 |
| F11 (target) | 120 | 10 | 10 | 0.10 | 6.0 |

The results demonstrate that variations of the critical formulation parameters within the studied ranges have no significant impact on the mAb1 formulation stability. The mAb1 formulations are robust with regards to general quality attributes within the tested formulation composition ranges.

The full factorial PAR studies with long-term shelf-life stability demonstrated that variation in the critical formulation parameters, within the range studies, did not affect mAb1 drug product quality. The stability of mAb1 formulations to stress was unaffected by a ±10% change in mAb1 concentration, a ±20% change in sucrose, and/or a ±0.3 pH unit change relative to the control mAb1 concentration. Specifically, the stability of mAb1 formulations stored at 5° C. for 12 months were unaffected by a ±10% variation in protein concentration, a ±20% variation in sucrose, and/or histidine concentration, and/or ±50% variation in polysorbate 80 concentration, and/or a ±0.3 pH unit variation (for 2, 10 and 20 mg/ml concentrations). The stability of the 120 mg/ml formulations was unaffected by variation in antibody or excipient concentration for at least a month, according to current results.

The robustness of mAb1 formulations was demonstrated by the PAR studies. Overall, the results from the PAR studies supported that variability in the compositions of the mAb1 formulation within the ranges studied would not adversely impact the stability of the mAb1 DP under the recommended storage conditions (2 to 8° C.).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Sequences
SEQ ID NO: 1
EVQLVESGGGLVQPGGSLRLSCAASGFTFSNFWMTWVRQAPGKGLEWVANMNQDGSEKYYVDSVKGRF
TISRDNAKSSLYLQMNSLRAEDTAVYYCARDREYCISTSCYDDFDYWGQGTLVTVSS
SEQ ID NO: 2
GFTFSNFW
SEQ ID NO: 3
MNQDGSEK
SEQ ID NO: 4
ARDREYCISTSCYDDFDY
SEQ ID NO: 5
DIQMTQSPSSLSASVGDRVTITCRASQSISSYLNWYQQKPGKAPKLLIYAASSLQSGVPSRFSGSGSG
TDFTLTISSLQPEDFATYYCQQSYSTPPITFGQGTRLEIK
SEQ ID NO: 6
QSISSY
SEQ ID NO: 7
AAS
SEQ ID NO: 8
QQSYSTPPIT
SEQ ID NO: 9
EVQLVESGGGLVQPGRSLRLSCAASGFTFDDYSMHWVRQAPGKGLEWVSGISWNSGSKGYADSVKGRF
TISRDNAKNSLYLQMNSLRAEDTALYYCAKYGSGYGKFYHYGLDVWGQGTTVTVSS
SEQ ID NO: 10
GFTFDDYS
SEQ ID NO: 11
ISWNSGSK
SEQ ID NO: 12
AKYGSGYGKFYHYGLDV
SEQ ID NO: 13
EVQLVESGGGLVQPGGSLRLSCAASGFTFSNFWMTWVRQAPGKGLEWVANMNQDGSEKYYVDSVKGRF
TISRDNAKSSLYLQMNSLRAEDTAVYYCARDREYCISTSCYDDFDYWGQGTLVTVSSASTKGPSVFPL
APCSRSTSESTAALGCLVKDYFPEPVTVSWNSGALTSGVHTFPAVLQSSGLYSLSSVVTVPSSSLGTK
TYTCNVDHKPSNTKVDKRVESKYGPPCPPCPAPPVAGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
SEQ ID NQ: 14
EVQLVESGGGLVQPGRSLRLSCAASGFTFDDYSMHWVRQAPGKGLEWVSGISWNSGSKGYADSVKGRF
TISRDNAKNSLYLQMNSLRAEDTALYYCAKYGSGYGKFYHYGLDVWGQGTTVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPPVAGPSVFLFPPKPKDTLMISRTPEVTCVVVDVSQE
DPEVQFNWYVDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPPVLDSDGSFF
LYSRLTVDKSRWQEGNVFSCSVMHEALHNRFTQKSLSLSPGK
SEQ ID NQ: 15
DIQMTQSPSSLSASVGDRVTITCRASQSISSYLNWYQQKPGKAPKLLIYAASSLQSGVPSRFSGSGSG
TDFTLTISSLQPEDFATYYCQQSYSTPPITFGQGTRLEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSS
PVTKSFNRGEC
SEQ ID NQ: 16
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPPVAGPSVFLFPPKPKDTLMISRTP
EVTCVVVDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGK
SEQ ID NQ: 17
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPPVAGPSVFLFPPKPKDTLMISRTP
EVTCVVVDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNRFTQKSLSLSPGK
SEQ ID NQ: 18
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPPVAGPSVFLFPPKPKDTLMIS
RTPEVTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NQ: 19
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPPVAGPSVFLFPPKPKDTLMIS
RTPEVTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNRFTQKSLSLSPGK
SEQ ID NQ: 20
RTVAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC

1. A stable liquid pharmaceutical formulation comprising:
(a) a bispecific antibody comprising a first antigen-binding domain that binds specifically to human BCMA and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 2, 3 and 4, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 6, 7 and 8;
(b) a buffer comprising histidine;
(c) an organic co-solvent comprising polysorbate; and
(d) a stabilizer comprising sucrose;
wherein the formulation has a pH of 6.0±0.3.

2. The pharmaceutical formulation of claim 1, wherein:
the antibody concentration is from 1 mg/ml±0.1 mg/ml to 200 mg/ml±20 mg/ml;
the antibody concentration is from 2 mg/ml±0.2 mg/ml to 125 mg/ml±12.5 mg/ml;
the antibody concentration is 2 mg/ml±0.2 mg/ml;
the antibody concentration is 10 mg/ml±1 mg/ml;
the antibody concentration is 20 mg/ml±2 mg/ml; or
the antibody concentration is 120 mg/ml±12.0 mg/ml.

3-7. (canceled)

8. The pharmaceutical formulation of claim 1, wherein:
the histidine buffer concentration is from 2 mM±0.4 mM to 20 mM±4 mM;
the histidine buffer concentration is from 5 mM±1 mM to 15 mM±3 mM;
the histidine buffer concentration is from 8 mM to 12 mM; or
the histidine buffer concentration is 10 mM±2 mM.

9-11. (canceled)

12. The pharmaceutical formulation of claim 1, wherein:
the polysorbate concentration is from 0.01%±0.005% to 0.5%±0.25% w/v;
the polysorbate concentration is from 0.05%±0.025% to 0.15%±0.025% w/v;
the polysorbate concentration if from 0.05% to 0.15% w/v; or
the polysorbate concentration is 0.1%±0.05% w/v.

13-15. (canceled)

16. The pharmaceutical formulation of claim 1, wherein the polysorbate is polysorbate 80.

17. (canceled)

18. The pharmaceutical formulation of claim 1, wherein:
the sucrose concentration is from 5%±1% to 20%±4% w/v;
the sucrose concentration is from 7%±0.5% to 12%±0.5% w/v;
the sucrose concentration is from 8% to 12% w/v; or
the sucrose concentration is 10%±2% w/v.

19-21. (canceled)

22. The pharmaceutical formulation of claim 1 comprising:
(a) 2 mg/ml±0.4 mg/ml antibody,
(b) from 5 mM±1 mM to 15 mM±3 mM histidine buffer,
(c) from 0.05%±0.025% to 0.15%±0.025% w/v polysorbate, and
(d) from 5%±1% to 15%±3% w/v sucrose,
at pH 6.0±0.3.

23. The pharmaceutical formulation of claim 22 comprising:
(a) 2 mg/ml±0.4 mg/ml antibody,
(b) 10 mM±2 mM histidine buffer,
(c) 0.1%±0.05% w/v polysorbate, and
(d) 10%±2% w/v sucrose,
at pH 6.0±0.3.

24. The pharmaceutical formulation of claim 1 comprising:
(a) 10 mg/ml±1 mg/ml antibody,
(b) from 5 mM±1 mM to 15 mM±3 mM histidine buffer,
(c) from 0.05%±0.025% to 0.15%±0.025% w/v polysorbate, and
(d) from 5%±1% to 15%±3% w/v sucrose,
at pH 6.0±0.3.

25. The pharmaceutical formulation of claim 24 comprising:
(a) 10 mg/ml±1 mg/ml antibody,
(b) 10 mM±2 mM histidine buffer,
(c) 0.1%±0.05% w/v polysorbate, and
(d) 10%±2% w/v sucrose,
at pH 6.0±0.3.

26. The pharmaceutical formulation of claim 1 comprising:
(a) 20 mg/ml±2 mg/ml antibody,
(b) from 5 mM±1 mM to 15 mM±3 mM histidine buffer,
(c) from 0.05%±0.025% to 0.15%±0.025% w/v polysorbate, and
(d) from 5%±1% to 15%±3% w/v sucrose,
at pH 6.0±0.3.

27. The pharmaceutical formulation of claim 26 comprising:
(a) 20 mg/ml±2 mg/ml antibody,
(b) 10 mM±2 mM histidine buffer,
(c) 0.1%±0.05% w/v polysorbate, and
(d) 10%±2% w/v sucrose,
at pH 6.0±0.3.

28. The pharmaceutical formulation of claim 1 comprising:
(a) 120 mg/ml±12 mg/ml antibody,
(b) from 5 mM±1 mM to 15 mM±3 mM histidine buffer,
(c) from 0.05%±0.025% to 0.15%±0.025% w/v polysorbate, and
(d) from 5%±1% to 15%±3% w/v sucrose,
at pH 6.0±0.3.

29. The pharmaceutical formulation of claim 28 comprising:
(a) 120 mg/ml±12 mg/ml antibody,
(b) 10 mM±2 mM histidine buffer,
(c) 0.1%±0.05% w/v polysorbate, and
(d) 10%±2% w/v sucrose,
at pH 6.0±0.3.

30. (canceled)

31. The pharmaceutical formulation of claim 1, comprising 2 mg/ml bispecific antibody, 0.7 mg/ml histidine, 1.1 mg/ml L-histidine hydrochloride, 100 mg/ml sucrose, 1 mg/ml polysorbate 80, and water for injection.

32. The pharmaceutical formulation of claim 1, consisting essentially of 2 mg/ml bispecific antibody, 0.7 mg/ml histidine, 1.1 mg/ml L-histidine hydrochloride, 100 mg/ml sucrose, 1 mg/ml polysorbate 80, and water for injection.

33. The pharmaceutical formulation of claim 1, consisting of 2 mg/ml bispecific antibody, 0.7 mg/ml histidine, 1.1 mg/ml L-histidine hydrochloride, 100 mg/ml sucrose, 1 mg/ml polysorbate 80, and water for injection.

34. The pharmaceutical formulation of claim 1, comprising 20 mg/ml bispecific antibody, 0.7 mg/ml histidine, 1.1 mg/ml L-histidine hydrochloride, 100 mg/ml sucrose, 1 mg/ml polysorbate 80, and water for injection.

35. The pharmaceutical formulation of claim 1, consisting essentially of 20 mg/ml bispecific antibody, 0.7 mg/ml histidine, 1.1 mg/ml L-histidine hydrochloride, 100 mg/ml sucrose, 1 mg/ml polysorbate 80, and water for injection.

36. The pharmaceutical formulation of claim 1, consisting of 20 mg/ml bispecific antibody, 0.7 mg/ml histidine, 1.1 mg/ml L-histidine hydrochloride, 100 mg/ml sucrose, 1 mg/ml polysorbate 80, and water for injection.

37. The pharmaceutical formulation of claim 1, wherein:
the formulation contains no more than 2% high molecular weight (HMW) species after 6 months of storage at 5° C., as determined by SE-UPLC;
the formulation contains no more than 4% high molecular weight (HMW) species after 6 months of storage at 25° C. and 60% relative humidity, as determined by SE-UPLC;
the formulation contains no more than 1.5% high molecular weight (HMW) species after 9 months of storage at −30° C., as determined by SE-UPLC; or
the formulation contains no more than 1.5% high molecular weight (HMW) species after 9 months of storage at −80° C., as determined by SE-UPLC.

38-40. (canceled)

41. The pharmaceutical formulation of claim 1, wherein:
the first antigen-binding domain comprises a HCVR with at least 90% identity to the amino acid sequence of SEQ ID NO: 1 and a LCVR with at least 90% identity to the amino acid sequence of SEQ ID NO: 5, and the second antigen-binding domain comprises a HCVR with at least 90% identity to the amino acid sequence of SEQ ID NO: 9 and a LCVR with at least 90% identity to the amino acid sequence of SEQ ID NO: 5;
the first antigen-binding domain comprises a HCVR with at least 95% identity to the amino acid sequence of SEQ ID NO: 1 and a LCVR with at least 95% identity to the amino acid sequence of SEQ ID NO: 5, and the second antigen-binding domain comprises a HCVR with at least 95% identity to the amino acid sequence of SEQ ID NO: 9 and a LCVR with at least 95% identity to the amino acid sequence of SEQ ID NO: 5; or
the first antigen-binding domain comprises a HCVR with at least 99% identity to the amino acid sequence of SEQ ID NO: 1 and a LCVR with at least 99% identity to the amino acid sequence of SEQ ID NO: 5, and the second antigen-binding domain comprises a HCVR with at least 99% identity to the amino acid sequence of SEQ ID NO: 9 and a LCVR with at least 99% identity to the amino acid sequence of SEQ ID NO: 5.

42-43. (canceled)

44. The pharmaceutical formulation of claim 1, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5.

45. A stable liquid pharmaceutical formulation comprising:
(a) 2 mg/ml±0.4 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5;
(b) 10 mM±2 mM histidine buffer, pH 6.0±0.3,
(c) 0.1%±0.05% w/v polysorbate 80, and
(d) 10%±2% w/v sucrose.

46. A stable liquid pharmaceutical formulation comprising:
(a) 10 mg/ml±2 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5;
(b) 10 mM±2 mM histidine buffer, pH 6.0±0.3,
(c) 0.1%±0.05% w/v polysorbate 80, and
(d) 10%±2% w/v sucrose.

47. A stable pharmaceutical formulation comprising:
(a) 20 mg/ml±4 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5;
(b) 10 mM±2 mM histidine buffer, pH 6.0±0.3,
(c) 0.1%±0.05% w/v polysorbate 80, and
(d) 10%±2% w/v sucrose.

48. A stable pharmaceutical formulation comprising:
(a) 120 mg/ml±12 mg/ml of a bispecific antibody comprising a first antigen-binding domain that binds specifically to human BCMA and a second antigen-binding domain that binds specifically to human CD3, wherein the first antigen-binding domain comprises a HCVR comprising the amino acid sequence of SEQ ID NO: 1 and a LCVR comprising the amino acid sequence of SEQ ID NO: 5, and the second antigen-binding domain comprises a HCVR comprising the amino acid sequence of SEQ ID NO: 9 and a LCVR comprising the amino acid sequence of SEQ ID NO: 5;
(b) 10 mM±2 mM histidine buffer, pH 6.0±0.3,
(c) 0.1%±0.05% w/v polysorbate 80, and
(d) 10%±2% w/v sucrose.

49. The pharmaceutical formulation of claim 1, wherein;
the antibody comprises a human IgG heavy chain constant region attached, respectively, to the HCVR of each of the first antigen-binding domain and the second antigen-binding domain;
the antibody comprises a human IgG1 heavy chain constant region attached, respectively, to the HCVR of each of the first antigen-binding domain and the second antigen-binding domain; or
the antibody comprises a human IgG4 heavy chain constant region attached, respectively, to the HCVR of each of the first antigen-binding domain and the second antigen-binding domain.

50-51. (canceled)

52. The pharmaceutical formulation of claim 49, wherein;
the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification;
the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification, wherein the modification comprises a H435R substitution (EU numbering) in a heavy chain of isotype IgG1 or IgG4; or
the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification, wherein the modification comprises a H435R substitution and a Y436F substitution (EU numbering) in a heavy chain of isotype IgG1 or IgG4.

53-57. (canceled)

58. The pharmaceutical formulation of claim 41, wherein the antibody comprises a first heavy chain containing the HCVR of the first antigen-binding domain and a second heavy chain containing the HCVR of the second antigen-binding domain, wherein the first heavy chain comprises residues 1-450 of the amino acid sequence of SEQ ID NO: 13 and the second heavy chain comprises residues 1-449 of the amino acid sequence of SEQ ID NO: 14.

59. The pharmaceutical formulation of claim 58, wherein the antibody comprises a common light chain containing the LCVR of the first and second antigen-binding domains, wherein the common light chain comprises the amino acid sequence of SEQ ID NO: 15.

60. The pharmaceutical formulation of claim 45, comprising 2 mg/ml bispecific antibody, 0.7 mg/ml histidine, 1.1 mg/ml L-histidine hydrochloride, 100 mg/ml sucrose, 1 mg/ml polysorbate 80, and water for injection, wherein the bispecific antibody is linvoseltamab.

61. The pharmaceutical formulation of claim 45, consisting essentially of 2 mg/ml bispecific antibody, 0.7 mg/ml histidine, 1.1 mg/ml L-histidine hydrochloride, 100 mg/ml sucrose, 1 mg/ml polysorbate 80, and water for injection, wherein the bispecific antibody is linvoseltamab.

62. The pharmaceutical formulation of claim 45, consisting of 2 mg/ml bispecific antibody, 0.7 mg/ml histidine, 1.1 mg/ml L-histidine hydrochloride, 100 mg/ml sucrose, 1 mg/ml polysorbate 80, and water for injection, wherein the bispecific antibody is linvoseltamab.

63. The pharmaceutical formulation of claim 47, comprising 20 mg/ml bispecific antibody, 0.7 mg/ml histidine, 1.1 mg/ml L-histidine hydrochloride, 100 mg/ml sucrose, 1 mg/ml polysorbate 80, and water for injection, wherein the bispecific antibody is linvoseltamab.

64. The pharmaceutical formulation of claim 47, consisting essentially of 20 mg/ml bispecific antibody, 0.7 mg/ml histidine, 1.1 mg/ml L-histidine hydrochloride, 100 mg/ml sucrose, 1 mg/ml polysorbate 80, and water for injection, wherein the bispecific antibody is linvoseltamab.

65. The pharmaceutical formulation of claim 47, consisting of 20 mg/ml bispecific antibody, 0.7 mg/ml histidine, 1.1 mg/ml L-histidine hydrochloride, 100 mg/ml sucrose, 1 mg/ml polysorbate 80, and water for injection, wherein the bispecific antibody is linvoseltamab.

66. (canceled)

67. A pharmaceutical composition, wherein the composition comprises the pharmaceutical formulation of claim 1, and the composition is contained in a container.

68. The pharmaceutical composition of claim 67, wherein;
the container is a vial;
the container is a vial, and the vial is a 5 ml, 6 ml, 10 ml or 20 ml Type 1 clear glass vial;
the container is a syringe;
the container is a syringe, and the syringe is low-tungsten glass;
the container is a prefilled syringe; or
the composition is contained in an autoinjector.

69-73. (canceled)

74. A kit comprising (i) a container containing a composition comprising the pharmaceutical formulation of claim 1, and instructions for use of the composition.

75. The kit of claim 74, wherein:
the container is a glass vial;
the container is a prefilled syringe;
the container is an autoinjector.

76-77. (canceled)

78. The kit of claim 74, wherein the instructions recite subcutaneous administration of the composition, or the instructions recite intravenous administration of the composition.

79. (canceled)

80. A unit dosage form comprising the pharmaceutical formulation of claim 1, wherein the antibody is present in an amount of from 0.1 mg to 500 mg.

81. The unit dosage form of claim 80, wherein;
the antibody is present in an amount of from 5 to 250 mg;
the antibody is present in an amount of from 100 to 400 mg;
the unit dosage form is a glass vial;
the unit dosage form is a prefilled syringe; or
the unit dosage form is an autoinjector.

82-85. (canceled)

86. A container containing a composition comprising the pharmaceutical formulation of claim 1.

87. The container of claim 86 that is a glass vial, a prefilled syringe, or an autoinjector.

88-89. (canceled)
